(19) United States
(12) Patent Application Publication
(10) Pub. No.: US 2024/0115739 A1
(43) Pub. Date: Apr. 11, 2024

(54) SYNTHETIC CAS12A FOR ENHANCED MULTIPLEX GENE CONTROL AND EDITING

(71) Applicant: The Board of Trustees of the Leland Stanford Junior University, Stanford, CA (US)

(72) Inventors: Lei S. QI, Stanford, CA (US); Lucie GUO, Stanford, CA (US); Hannah KEMPTON, Stanford, CA (US)

(21) Appl. No.: 18/546,177

(22) PCT Filed: Feb. 11, 2022
(86) PCT No.: PCT/US22/16223

§ 371 Date: Aug. 11, 2023

Related U.S. Application Data
(60) Provisional application No. 63/148,652, filed on Feb. 12, 2021.

(51) Int. Cl. A61K 48/00; C12N 9/22; C12N 15/11; C12N 15/86

(52) U.S. Cl. CPC A61K 48/0058; C12N 9/22; C12N 15/11; C12N 15/86; C12N 2310/20; C12N 2320/32; C12N 2750/14143

(57) ABSTRACT

The present disclosure generally relates to engineered Cluster Regularly Interspaced Short Palindromic Repeat (CRISPR)-associated (Cas) 12a proteins and system, and methods for use in gene editing and gene modulation for application to gene therapy. Related systems and methods of gene modulation are also disclosed.

### CROSS-REFERENCE TO RELATED APPLICATIONS

This application claims the benefit of U.S. Provisional Patent Application No. 63/148,652, filed Feb. 12, 2021, which is incorporated herein by reference in its entirety.

### GRANT INFORMATION

This invention was made with Government support under T32-EY020485 awarded by National Institutes of Health. The Government has certain rights in this invention.

### FIELD OF THE DISCLOSURE

The present disclosure generally relates to engineered Cluster Regularly Interspaced Short Palindromic Repeat (CRISPR)-associated (Cas) 12a proteins and system, and methods for use in gene editing and gene modulation for application to gene therapy. Related systems and methods of gene modulation are also disclosed.

### BACKGROUND OF THE DISCLOSURE

Gene therapy has proved helpful for incurable diseases, and therapies utilizing CRISPR-based gene editing are entering clinical trials. However, gene therapy is currently limited to inherited and monogenic conditions, and there is an unmet need to expand the scope of gene therapy beyond monogenic diseases, to more common polygenic, complex, and degenerative conditions.

While adeno-associated viruses (AAVs) have emerged as a safe vehicle for gene therapy delivery, its ability to accommodate polygenic gene therapy will require large payloads that exceed packaging limitations of AAVs. Meanwhile, CRISPR based technologies hold great potential for genome engineering in a multiplex fashion. CRISPR/Cas enzymes have been widely used for genetic modulation in mammalian cells. For example, Cas9 has been used broadly for gene editing and gene therapy applications. However, Cas9 is large, immunogenic, and more importantly, less efficient for controlling or editing more than 1-2 genes.

To address this limitation of Cas9, Cas12a has emerged as a new system with its ability to process multiple CRISPR RNAs (crRNAs) from a long array on a single transcript, driven by a single promoter. However, the utility of Cas12a for in vivo applications is hampered by its relatively lower activity compared to Cas9, especially when applied to multiplexing. Improvements in Cas12a activity to enable more efficient gene editing and gene modulation to therapeutically relevant levels would enable more robust multiplex gene therapy application.

To solve this problem, the present disclosure provides engineered Cas12a proteins (such as vgdCas12a) with dramatically enhanced efficacy in CRISPR activation, particularly at lower crRNA conditions, through structure-based protein engineering.

### BRIEF SUMMARY OF THE DISCLOSURE

Provided herein, among others, is an engineered Cluster Regularly Interspaced Short Palindromic Repeat (CRISPR)-associated (Cas) 12a protein. In some embodiments, the engineered Cas12a protein comprises a sequence that is at least 80% identical to the amino acid sequence of SEQ ID NO: 1 or 2. In certain embodiments, the engineered Cas12a protein comprises one or more mutations selected from the list consisting of D122R, E125R, D156R, E159R, D235R, E257R, E292R, D350R, E894R, D952R, and E981R. In certain embodiments, the engineered Cas12a protein comprises one or more mutations selected from the list consisting of D156R, D235R, E292R, and D350R.

In some embodiments, the engineered Cas12a protein comprises at least two, three, or four mutations. In certain embodiments, in the engineered Cas12a protein comprises the mutations of D156R and E292R. In other embodiments, the engineered Cas12a protein comprises the mutations of D156R and D350R. In some embodiments, the engineered Cas12a protein comprises the mutations of D156R, E292R, and D235R. In some embodiments, the engineered Cas12a protein comprises the mutations of D156R, E292R, and D350R. In other embodiments, the engineered Cas12a protein comprises the mutations of D156R, D235R, E292R, and D350R.

In some embodiments, the engineered Cas12a protein exhibits improved activation compared to the wild type (WT) Cas12a protein. In other embodiments, the engineered Cas12a protein exhibits improved repression compared to the WT Cas12a protein. In some embodiments, the engineered Cas12a protein exhibits enhanced regulatory effect compared to the WT Cas12a protein. In other embodiments, the engineered Cas12a protein exhibits improved epigenetic modifications compared to the WT Cas12a protein. In some embodiments, the engineered Cas12a protein exhibits improved gene knockout, knockin, and mutagenesis compared to the WT Cas12a protein. In other embodiments, the engineered Cas12a protein exhibits improved gene editing of single or multiple bases compared to the WT Cas12a protein. In still other embodiments, the engineered Cas12a protein exhibits improved gene prime editing compared to the wild type (WT) Cas12a protein.

In some embodiments, the engineered Cas12a protein is less susceptibility to variations in crRNA concentration compared to the WT Cas12a protein. In certain embodiments, the engineered Cas12a protein exhibits increased level of activation under crRNA:Cas12a ratio of or lower compared to the WT Cas12a protein.

In another aspect, the present disclosure also provides a nucleic acid encoding the engineered Cas12a protein described herein. Further, the present disclosure also provides a vector comprising the nucleic acid described herein. in some embodiments, the vector further comprises a promoter.

The present disclosure further provides an engineered Cas12a system. In some embodiments, the engineered Cas12a system comprises: (a) one or more CRISPR RNAs (crRNAs) or a nucleic acid encoding each of the one or more crRNAs; and (b) the engineered Cas12a protein of any one of the preceding claims or a nucleic acid encoding the engineered Cas12a protein thereof. In other embodiments, each of the one or more crRNAs of the engineered Cas12a system comprises a repeat sequence and a spacer.

In some embodiments, each spacer is configured to hybridize to a target nucleic acid. In some embodiments, each spacer in at least a portion of the one or more crRNAs is configured to hybridize to the same target nucleic acid. In some embodiments, each spacer in at least a portion of the one or more crRNAs is configured to hybridize to a different target nucleic acid. In other embodiments, each spacer in all of the one or more crRNAs is configured to hybridize to a different target nucleic acid. In some embodiments, the target nucleic acid is a DNA.

In some embodiments, the engineered Cas12a system comprises one or more expression vectors.

In some embodiments, the one or more crRNAs and the engineered Cas12a protein of the engineered Cas12a system are located in separate vectors. In other embodiments, the one or more crRNAs and the engineered Cas12a protein of the engineered Cas12a system are located in the same vector.

In some embodiments, the expression of the one or more crRNAs or the engineered Cas12a protein is driven by an RNA polymerase III promoter or an RNA polymerase II promoter. In certain embodiments, the RNA polymerase III promoter comprises the mouse U6 promoter, the human U6 promoter, the H1 promoter, and the 7SK promoter. In certain embodiments, the RNA polymerase II promoter comprises a CAG promoter, PGK promoter, CMV promoter, EF1α promoter, SV40 promoter, and Ubc promoter. In certain embodiments, the CAG promoter is synthetic. In some embodiments, the expression of the one or more crRNAs or the engineered Cas12a protein is driven by an inducible promoter. In certain embodiments, the inducible promoter comprises a TRE promoter.

In some exemplary embodiments, the one or more crRNAs and the engineered Cas12a protein are located in the same vector, and wherein the expression of the one or more crRNAs or the engineered Cas12a protein is driven by the same promoter. In other exemplary embodiments, the one or more crRNAs and the engineered Cas12a protein are located in the same vector, and wherein the expression of the one or more crRNAs or the engineered Cas12a protein is driven by different promoters.

Also provided herein, among others, is a method of modulating one or more target nucleic acids in a sample. In some embodiments, the method comprises contacting the sample with a plurality of the engineered Cas12a protein, or a plurality of the engineered Cas12a system, provided herein. In other embodiments, the method further comprises modulating the more than one target nucleic acids simultaneously. In some embodiments, the modulating results in transcriptional activation of the one or more target nucleic acids.

In some embodiments, the modulating results in transcriptional repression of the one or more target nucleic acids. In other embodiments, the modulating results in epigenetic modifications including targeted CpG methylation, histone H2, H3 or H4 methylation or acetylation of the one or more target nucleic acids. In some embodiments, the modulating results in editing single or multiple bases of the one or more target nucleic acids. In other embodiments, the modulating results in altered expression of the one or more target nucleic acids. In some embodiments, the modulating results in reprograming the lineage of the sample. In other embodiments, the modulating the target nucleic acid in the sample results in depletion of the one or more target nucleic acids.

In some embodiments, the one or more target nucleic acids comprise one or more nucleic acids encoding functional proteins. In other embodiments, the one or more target nucleic acids comprise one or more nucleic acids encoding transcriptional factors and/or metabolic enzymes. In some embodiments, the one or more target nucleic acids is derived from the genomic DNA, mitochondria DNA, chloroplast DNA, or viral DNA in host cells. In some embodiments, the sample comprises one or more cells. In other embodiments, the contacting of the method takes place in vitro or in vivo.

Further provided herein is a pharmaceutical composition. In some embodiments, the pharmaceutical composition comprises the engineered Cas12a protein, the nucleic acid, or the vector provided herein. In other embodiments, the present disclosure proses a pharmaceutical composition comprising the engineered Cas12a system described herein. In some embodiments, the pharmaceutical composition further comprises one or more pharmaceutically acceptable excipient.

Additionally, the present disclosure provided a method for treating a disorder in an individual in need thereof. In some embodiments, the method for treating comprises administering a therapeutically effective dose of the pharmaceutical composition provided herein. In some embodiments, the disorder is monogenic or polygenic. In other embodiments, the disorder comprises an inherited retinal degenerative disorder, an inherited optic nerve disorder, and a polygenic degenerative disease of the eye. In some embodiments, the inherited retinal degenerative disorder comprises Leber's congenital amaurosis and retinitis pigmentosa. In certain embodiments, the inherited optic nerve disorder comprises Leber's hereditary optic neuropathy and autosomal dominant optic neuropathy. In some embodiments, the polygenic degenerative disease of the eye comprises glaucoma and macular degeneration.

### BRIEF DESCRIPTION OF THE DRAWINGS

FIGS. 1A-1H show the systematic screening identifying combinatorial LbdCas12a mutants that outperform wildtype especially at low reactant conditions. FIG. 1A: Structure of LbCas12a (PDB 5XUS) showing the target DNA and all Glu and Asp residues within 10 Å of the target DNA. FIG. 1B: Schematic of constructs used for co-transfection to test CRISPR activation using a Tet crRNA driven by U6 promoter, with various dCas12a mutants in a HEK293T reporter cell line stably expressing GFP driven by the inducible TRE3G promoter. FIG. 1C: GFP fluorescence in reporter cell line for WT dCas12a vs. various dCas12a mutants. Fold changes were calculated relative to non-targeting crLacZ. For ease of visualization, dotted line in each graph is drawn at the level of WT. FIG. 1D: Representative flow cytometry histogram of GFP intensity, comparing untransfected vs. transfected cells, showing threshold for BFP+ and subset of “low BFP” cells. FIG. 1E: GFP fluorescence in the “low BFP” cells, comparing WT dCas12a, single mutants, as well as combinatorial mutants consisting of the several most potent single mutations from FIG. 1C. The quadruple mutant (D156R+D235R+E292R+D350R) is henceforth referred to as “very good dCas12a” (vgdCas12a). Fold changes were calculated relative to non-targeting crLacZ. For ease of visualization, dotted lines in the graph are drawn at the level of the WT mutant as well as the single D156R mutant. FIG. 1F: Schematic of constructs used for co-transfection to test CRISPR-activation of a Tet crRNA driven by a Pol III promoter (CAG) in the same reporter cell line as FIG. 1C, comparing WT dCas12a vs. mutants including vgdCas12a. FIG. 1G: GFP fluorescence for WT dCas12a vs. various dCas12a mutants, both at 1:1 dCas12a:crRNA ratio (left panel), and 1:0.2 dCas12a:crRNA ratio (right panel). FIG. 1H: In parental HEK293T cells, hyperdCas12a vs. WT dCas12a and crTet were co-transfected with a third plasmid containing a truncated TRE3G promoter that contains a single TetO element preceded by 27 various PAMs. Cells were gated for mCherry+ and low BFP+. Fold activation changes were calculated relative to non-targeting crLacZ. For ease of visualization, dotted line is drawn at the level of the non-targeting crRNA.

FIGS. 2A-2O show that VgdCas12a outperforms WT dCas12a in multiple applications. FIG. 2A: Schematic of constructs used for co-transfection to test GFP knockout by gene editing, in a HEK293T reporter cell line stably expressing GFP driven by SV40 promoter. A crRNA targeting GFP is used. FIG. 2B: GFP fluorescence in the assay described in panel c, comparing nuclease-active WT Cas12a vs. vgCas12a. FIG. 2C: Schematic of constructs used for co-transfection to test CRISPR-repression in the same reporter cell line as FIG. 2A, in which either WT dCas12a or vgdCas12a is fused to the transcriptional repressor KRAB. FIG. 2D: GFP fluorescence in the CRISPRi assay described in FIG. 2C, comparing WT dCas12a-KRAB vs. vgdCas12a-KRAB. FIG. 2E: Base editing assay comparing dCas12a vs. vgdCas12a fused to the adenine base editor ABE8, in a cell line in which base editing would remove an internal stop codon within GFP to allow for translation of the full-length protein. FIG. 2F: GFP fluorescence results in the base editing assay described in FIG. 2E. FIG. 2G: Quantitation of percentage of GFP+ cells in the base editing assay described in FIG. 2E. FIG. 2H, Base editing assay comparing dCas12a vs. vgdCas12a for an endogenous gene target (Klf4). FIGS. 2I-2J: Schematic (FIG. 2I) and results (FIG. 2J) or dual-GFP reporter assay, in which removal of both stop codons in a single GFP gene (which requires targeting by two crRNAs) is required for translation of full-length GFP. NT=nontargeting. FIG. 2K: Schematic of AAV constructs for in vivo gene editing. AAV-enAsCas12a exceeds the AAV packaging limit (>4.7 kb). FIG. 2L: Schematic of AAVs delivered by intravitreal injection, where AAV-hyperCas12a+AAV-crYFP is delivered into one eye while AAV-WT Cas12a+AAV-crYFP is delivered to the fellow eye as internal control. Mice were sacrificed 10 weeks later for retinal histology. FIG. 2M: Immunohistochemistry of retinal wet mounts. Dotted circle highlighted mCherry+/HA+ retina cells missing YFP expression. Dotted circles highlight cells with YFP knockout. Scale bars (white line), 100 μm. Scale bars within insets (yellow line), 20 μm. FIG. 2N: Quantification of YFP fluorescence in mCherry+ cells in each mouse by automated segmentation analyses. The data for all 6 mice are displayed, which are 6 independent biological replicates. For each mouse, 250-800 cells were analyzed. For box-and-whisker plots, the box shows 25-75% (with bar at median, dot at mean), and whiskers encompass 10-90%, with individual data points 382 shown for the lowest and highest 10% of each dataset. FIG. 2O: The mean YFP fluorescence (left), HA signal (middle) and mCherry fluorescence (right) for WT Cas12a vs. hyperCas12a for each mouse as measured by automated segmentation analysis. Mean±s.d. and individual data points shown for n=6 animals. The P-values were calculated using a paired two-tailed Student's t-test; **p=0.0078; ns, non-significant. For the YFP graph, blue dotted lines are drawn to connect values for each mouse to facilitate ease of comparison of this paired dataset.

FIG. 3 shows vgdCas12a targeting has minimal off-targeting effects. FKPM (Fragments Per Kilobase Million) plots of genome-scale RNA sequencing (RNA-seq). Plasmids with dCas12a-miniVPR (WT or vgdCas12a) and crRNA to TRE3G promoter were co-transfected into HEK293T reporter cell line stably expressing TRE3G-GFP (per FIG. 1B). The GFP gene is highlighted in green.

FIGS. 4A-4I show that VgdCas12a enables multiplex activation of endogenous genes. FIG. 4A: Schematic of experiment. Mouse P19 cells were co-transfected (with plasmids shown in right panel), then selected with puromycin and hygromycin 24 hours after transfection. Cells were collected for analysis 72 hours after transfection. FIGS. 4B-4D: Schematics of crRNAs targeting promoters of Oct4 (FIG. 4B), Sox2 (FIG. 4C), and Klf4 (FIG. 4D), as well as transcriptional activation of each target gene by qPCR by WT dCas12a vs. vgdCas12a, relative to non-targeting crRNA. TSS=transcriptional start site. FIG. 4E: Schematic constructs used for testing multiplex activation by WT dCas12a vs vgdCas12a, including the 7-crRNA array driven by the U6 promoter. FIG. 4F: Multiplex transcriptional activation of each target gene by qPCR, relative to non-targeting crRNA. FIGS. 4G-4H: Immunostaining of cells from experiment in FIG. 4E, with antibodies targeting endogenous Sox2 (FIG. 4G), Oct4 (FIG. 4G), or Klf4 (FIG. 4H). FIG. 4I: hyperdcas12 outperforms enAsdCas12a for multiplex activation in mouse P19 cells.

FIGS. 5A-5E show the in vivo CRISPR-activation by vgdCas12a. FIG. 5A: Schematic of constructs and experiment used for in vivo plasmid electroporation in postnatal mouse retina. CAG-GFP is used to mark the electroporated patch. Wildtype CD-1 pups are electroporated on day of birth, and sacrificed at day 14 of life to access retinal histology. FIGS. 5B and 5D: Representative retinal slices. Note that GFP signal marks the boundary of the electroporated patch, thus the area that did not receive electroporated plasmids serves as an internal control that aids in interpreting the specificity of immunostaining. HA marks the cells that received the plasmid with vgdCas12a and crRNA array. Immunostaining was performed with antibody to Klf4 (FIG. 5B) or Sox2 (FIG. 5D), indicating cells that achieved CRISPR activation. Insets (right panels) highlight nuclei that demonstrate colocalization of GFP, HA and the target genes. FIGS. 5C and 5E: quantification of percentage of Klf4 (FIG. 5C) and Sox2 (FIG. 5E) cells among HA+ cells for the non-targeting (NT) crRNA and 6-crRNA array conditions. ONL, outer nuclear layer. OPL, outer plexiform layer. INL, inner nuclear layer. IPL, inner plexiform layer. GCL, ganglion cell layer. Scale bar indicates 100 μm.

FIGS. 6A-6D show that multiplexed CRISPR activation by vgdCas12a induces retinal progenitor cell migration. FIG. 6A: vgdCas12a activation of endogenous Oct4/Sox2/Klf4 induces migration of retinal neurons to ganglion cell layer (GCL) and inner plexiform layer (IPL). ONL, outer nuclear layer. OPL, outer plexiform layer. INL, inner nuclear layer. IPL, inner plexiform layer. GCL, ganglion cell layer. FIG. 6B: characterization of percentage of HA+ cells in GCL, IPL, and INL for the non-targeting crRNA (the bars on the right for each group) and 6-crRNA array (the bars on the left for each group). FIG. 6C: vgdCas12a-mediated activation of endogenous Oct4/Sox2/Klf4 in retinal progenitor cells induces formation of Pax6+ cells. The yellow boxes show an inset with co-localized Pax6, HA and DAPI staining. FIG. 6D: vgdCas12a activation of endogenous Oct4/Sox2/Klf4 induces formation of ganglion-like cells as indicated by RBPMS expression colocalized with HA. Two insets from the slice are shown on the right. Scale bar indicates 100 μm.

FIGS. 7A-7C show relative expression levels of dCas12a (mCherry) and crRNA (BFP) across tested variants. FIG. 7A: Mean BFP fluorescence across the mutants tested in FIG. 1C. FIG. 7B: Mean mCherry fluorescence among mutants tested in FIG. 1C. FIG. 7C: Schematic of the LbCas12a protein domains and location of four of the most potent point mutants, with alignment across various Cas12a species.

FIGS. 8A-8E show tests of variants containing mutations of homologous residues to enAsCas12a. FIG. 8A: Alignment of the structure of LbCas12a and AsCas12a proteins and FIG. 8B: Alignment of peptide sequences encompassing mutations harbored by enAsCas12a, a previously reported enhanced variant of Cas12a from Acidaminococcus with the E174R/S542R/K548R mutations. We tested whether mutations of the homologous residues (D156R/G532R/K538R) in LbdCas12a improved its activity. FIG. 8C: Gating condition for BFP representing the low (bin 1), medium (bin 2), and high (bin 3) expression of crRNA in each population. FIG. 8D: Characterization of GFP activation for each bin across wildtype, single, double, and triple mutations of D156R/G532R/K538R. Interestingly, D156R combined with G532R and/or K538R did not achieve activation higher than the single D156R, in contrast to results with homologous residues in AsCas12a. FIG. 8E: As control, GFP activation using the variants mutants and a non-targeting crLacZ.

FIG. 9 shows optimization of NLS structure. It was previously shown that replacing the SV40 nuclear localization sequence (NLS) with the c-Myc NLS may improve knockout efficiency of AsCas12a. Here, we compared a dual SV40 NLS vs. a dual c-Myc NLS and show that while they achieve comparative efficiency for gene activation in bulk population, the dual c-Myc NLS conferred higher efficiency at lower reactant concentration of the crRNA-Cas12a complex (bin 1). We thus elected to use the dual c-Myc NLS for subsequent in vivo targeting.

FIG. 10 shows RNAseq replicates. Reproducibility of RNA-seq data showing FKPM (Fragments Per Kilobase Million) between two biological duplicates for each condition.

FIGS. 11A-11D shows characterization of transfection conditions of plasmids encoding the crRNA and dCas12a in P19 cells. FIG. 11A: Plasmids used for transfection. FIG. 11B: Schematic of experiment. Mouse P19 cells were co-transfected (with plasmids shown in right panel), then selected with puromycin and hygromycin at 24 h after transfection. Cells were collected for analysis 72 h after transfection. FIG. 11C: histograms showing percentage of BFP+ (crRNA) and mCherry+ (dCas12a) for non-transfected, non-selected, and Puro/Hygro selected cells. FIG. 11D: characterization of double BFP+/mCherry+ cells.

FIGS. 12A-12D show design and characterization of crRNAs for activating endogenous Oct4. FIG. 12A: Schematics of dCas12a crRNAs (red) targeting promoters of Oct4 and their relative position to known dCas9 sgRNAs that are functional (black) or non-functional (grey) in activating Oct4. Arrows indicate sense or antisense binding of crRNAs/sgRNAs to the target DNA. FIG. 12B: Immunostaining of Oct4 expression and their colocalization with BFP and mCherry. FIG. 12C: Magnification of the box highlighted in FIG. 12B. FIG. 12D: Immunostaining of Oct4 expression for most efficient crRNAs (O1, O2, O1+O2) and comparison with dCas9-miniVPR and a validated sgRNA (O127).

FIGS. 13A-13D shows design and characterization of crRNAs for activating endogenous Sox2. FIG. 13A: Schematics of dCas12a crRNAs (red) targeting promoters of Sox2 and their relative position to validated dCas9 sgRNAs. Arrows indicate sense or antisense binding of crRNAs/sgRNAs to the target DNA. FIG. 13B: Immunostaining of Sox2 expression from activation by various Sox2 single crRNAs compared to activation by dCas9-miniVPR (using a validated sgRNA, S84). FIGS. 13C-13D, Immunostaining of Sox2 expression and colocalization with BFP and mCherry for a pair of crRNAs (FIG. 13C) and a panel of ‘triplets’ of crRNAs (FIG. 13D), demonstrating synergy when multiple crRNAs are used in tandem.

FIGS. 14A-14B shows design and characterization of crRNAs for activating endogenous Klf4. FIG. 14A: Schematics of dCas12a crRNAs (red) targeting promoters of Klf4 and their relative position to known dCas9 sgRNAs that are functional (black) or non-functional (grey) in activating Klf4. Arrows indicate sense or antisense binding of crRNAs/sgRNAs to the target DNA. FIG. 14B: Immunostaining of Oct4 expression for selected crRNAs (K2, K4, K1+K2, K1+K4). The insets show colocalization between mCherry (vgdCas12a) and Klf4 immunostaining.

FIG. 15A-15C show characterization of vgdCas12a expression in mice retina in vivo. FIG. 15A: Schematic of constructs and experiment used for in vivo plasmid electroporation in postnatal mouse retina. CAG-GFP is used to mark the electroporated patch. Wildtype CD-1 pups are electroporated on day of birth and sacrificed at day 14 of life to access retinal histology. FIG. 15B: Representative retinal slices showing efficient dCas12a expression in vivo. Note that GFP signal marks the boundary of the electroporated patch, thus the area that did not receive electroporated plasmids serves as an internal control that aids in interpreting the specificity of immunostaining. mCherry marks the cells that received the plasmid with dCas12a. FIG. 15C: Magnification of the highlighted box in FIG. 15B. The images show adjusted GFP brightness and colocalization of mCherry and GFP.

FIGS. 16A-16B show in vivo Klf4 activation by vgdCas12a. FIG. 16A: Schematic of constructs and experiment used for in vivo plasmid electroporation in postnatal mouse retina. CAG-GFP is used to mark the electroporated patch. Wildtype CD-1 pups are electroporated on day of birth and sacrificed at day 14 of life to access retinal histology. FIG. 16B: Representative retinal slices for Klf4 activation. HA marks the cells that received the plasmid with vgdCas12a and crRNA array. Immunostaining was performed with antibody to Klf4, indicating cells that achieved CRISPR activation. Insets (right panels) highlight nuclei that demonstrate colocalization of GFP, HA and Klf4. The retinal slice is different from the ones shown in FIG. 6A.

FIG. 17 shows representative retinal slices for Oct4 activation. HA marks the cells that received the plasmid with vgdCas12a and crRNA array. Immunostaining was performed with antibody to Oct4. Only a few cells showed CRISPR activation of Oct4, indicating the relatively low efficiency for activating Oct4 (compared to Klf4 and Sox2). Insets (bottom panels) highlight nuclei that demonstrate colocalization of GFP, HA and Oct4.

FIGS. 18A-18C show the sequence alignments of the Cas12a nucleases described herein.

FIG. 19A-19L show In vivo multiplex gene activation by hyperdCas12a compared to dCas12a alternatives. FIGS. 19A-19I are representative retinal slices after in vivo electroporation with crRNA array and hyperdCas12a (FIGS. 19A, 19B, 19C), WT LbdCas12a (FIGS. 19D, 19E, 19F), or enAsdCas12a (FIGS. 19G, 19H, 19I)) to activate endogenous Sox2, Klf4 and Oct4 expression. Insets highlight HA+ cells in the inner nuclear layer (INL). ONL, outer nuclear layer. OPL, outer plexiform layer. INL, inner nuclear layer. IPL, inner plexiform layer. GCL, ganglion cell layer. Scale bar, 50 μm. FIGS. 19J-19L show Quantitative comparison of the percentage of Sox2+ cells (FIG. 19J), Klf4+ cells (FIG. 19K) and Oct4+ cells (FIG. 19L) among HA+ cells in INL layer in mouse retina electroporated with plasmids containing crRNA array and hyperdCas12a, WT dCas12a or enAsdCas12a. Value represent mean±s.d. and individual data points shown for 3-5 independent biological replicates. For J-K, p values were calculated using an unpaired two-tailed Student's t-443 test and are indicated on the graphs.

### DETAILED DESCRIPTION OF THE DISCLOSURE

Described and illustrated herein are engineered Cluster Regularly Interspaced Short Palindromic Repeat (CRISPR)-associated (Cas) 12a proteins and systems, nucleic acids, vectors, pharmaceutical compositions, and methods of using thereof.

CRISPR-Cas nucleases have revolutionized the field of gene editing. Alternative CRISPR nucleases beyond the most widely used Streptococcus pyogenes Cas9 (SpCas9) have greatly expanded the toolkit for gene modulation. Cas12a nucleases (also known as Cpf1), such as Acidaminococcus Cas12a (AsCas12a) and Lachnospiraceae bacterium Cas12a (LbCas12a), recognize T-rich PAMs and require only a short (generally about 23 nucleotide (nt)) CRISPR RNA (crRNA) with a spacer sequence of about 20 nt long. Furthermore, Cas12a enzymes possess their own RNAse activity, thus able to process a poly-crRNA transcript and enable multiplex targeting. This characteristic of Cas12a makes it powerful for multiplex gene modulation, including combinatorial genetic screening.

However, a major drawback of Cas12a is its decreased and more variable insertion and deletion (indel) efficiency compared to Cas9, which would limit its applicability in vivo where fewer copies of the crRNA-Cas complex would be delivered compared to in vitro delivery. While Cas12a has shown some utility in vivo, its editing efficiency in vivo has been shown to be significantly lower than all Cas9 orthologs. Although there are enhanced versions of AsCas12a, these enzymes have not yet been tested in vivo. Thus, even though Cas12a is a promising tool for epigenetic and transcriptional modulation, its utility for multiplex epigenetic modulation has not been demonstrated in vivo. Accordingly, the present disclosure solves these problems by providing higher-performance Cas12a variants specifically for in vivo multiplex epigenetic modulation.

The engineered Cas12a proteins and systems described herein enable simultaneous genome modulation at multiple genomic loci, thus paving the way for CRISPR-based treatment of polygenic diseases, which consist of a large proportion of human diseases. Without being bound by theory, as our capabilities in genetic diagnoses continues to expand at an unprecedented pace, especially with the increasing power and accessibility of next-generation sequencing technologies, there will likely be a concomitant demand for therapeutic strategies to combat polygenic genetic diseases as personalized medicine.

In some embodiments, the present disclosure demonstrates the superior CRISPR activation activity of vgdCas12a (also referred to herein as hyperdCas12a). Further, by way of example, the present disclosure demonstrates that the vgdCas12a provided herein is useful for additional Cas12a-based applications, including CRISPR repression and base editing. The present disclosure also demonstrates that the four activity-enhancing mutations provided herein, when introduced into the nuclease-active form of Cas12a, enhanced gene editing. Additionally, the present disclosure evaluates the specificity of CRISPR activation by vgdCas12a on a genome-wide scale, and demonstrates that CRISPR activation by vgdCas12a described herein is highly specific. In some exemplary embodiments, the present disclosure shows that the VgdCas12a described herein effectively activates endogenous genes and exhibits synergistic endogenous gene activation. In other exemplary embodiments, the present disclosure demonstrates the enhanced multiplex activation of endogenous genes driven by the vgdCas12a described herein. In additional exemplary embodiments, the present disclosure demonstrates the in vivo multiplex activation by vgdCas12a described herein in mouse retina directs retinal progenitor cell differentiation.

Moreover, the engineered Cas12a proteins and systems described herein can be useful as a platform for regenerative biology and therapy. For example, there is high interest in the direct reprogramming of lineage-determined cells from one cell fate to another, as therapeutic strategy for loss of a certain cell population in disease (for example, the fate conversion of glial cells in the retina to replace photoreceptor cells such as rods or cones, in degenerative diseases such as retinitis pigmentosa or macular degeneration). The engineered Cas12a proteins and systems described herein enable the simultaneous manipulation of the endogenous expression of a slew of fate-determining transcription factors, which will have wide applicability for regenerative biology. The engineered Cas12a proteins and systems described herein can further be used in an organoid context. Furthermore, the engineered Cas12a proteins and systems described herein are useful for cell therapy. For instance, recognition of tumor-associated antigens is a pillar of immunotherapy, and multiplex CRISPR activation (CRISPRa) can be used to augment the expression of tumor antigens, especially those that may be lowly expressed (or downregulated) at a level that would bypass an effective T-cell mediated response.

### I. Definitions

A “sample” as used here can be a biological sample including, without limitation, a cell, a tissue, fluid, or other composition in an organism. In some embodiments, the sample is a cell or a composition comprising a cell. In some embodiments, the cell is a mammalian cell, e.g., a human cell. In some embodiments, the sample comprises one or more cells.

The terms “subject” and “individual” are used interchangeably herein to refer to a vertebrate, preferably a mammal, more preferably a human. In some cases, a subject is a patient. Mammals include, but are not limited to, murines, simians, humans, farm animals, sport animals, and pets. Tissues, cells and their progeny of a biological entity obtained in vivo or cultured in vitro are also encompassed.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective dose” or “therapeutically effective dose” refers to the dose or amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the singular forms “a,” “an,” and “the” include both singular and plural referents unless the context clearly dictates otherwise. As used herein, “a” or “an” may mean one or more than one.

The term “optional” or “optionally” means that the subsequent described event, circumstance or substituent may or may not occur, and that the description includes instances where the event or circumstance occurs and instances where it does not.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes, such as variations of +/−10% or less, +/−1-5% or less, +/−1% or less, and +/−0.1% or less from the specified value. In determining whether a number is near to or approximately a specifically recited number, the near or approximating unrecited number may be a number which, in the context in which it is presented, provides the substantial equivalent of the specifically recited number.

### II. Compositions

The present disclosure provides, among others, engineered Cluster Regularly Interspaced Short Palindromic Repeat (CRISPR)-associated (Cas) 12a proteins.

As used herein, a CRISPR associated (“Cas”) nuclease refers to a protein encoded by a gene generally coupled, associated or close to or in the vicinity of flanking CRISPR loci, and further capable of introducing a double strand break into a target nucleic acid sequence (e.g., RNA or DNA). The terms “Cas nuclease” and “Cas protein” are used interchangeably herein. In some embodiments, a Cas protein is guided by a guide polynucleotide to recognize and introduce a double strand break at a specific target site into the genome of a cell. Upon recognition of a target sequence by a CRISPR RNA (also called crRNA), a Cas protein unwinds the DNA duplex in close proximity of the target sequence and cleaves both DNA strands or a target RNA strand, e.g., if the correct protospacer-adjacent motif (PAM) is approximately oriented at the 3′ end of the target sequence.

### Engineered Cas12a Proteins

In some embodiments, the Cas protein is a Cas12a. Cas12a is an RNA-programmable DNA endonuclease. Cas12a has intrinsic RNase activity that allows processing of its own crRNA array, enabling multigene editing from a single RNA transcript. Typically, a Cas12a nuclease binds double-stranded DNAs (dsDNA). Cas12a (also known as Cpf1), is a Class 2, Type V RNA-guided endonuclease from the CRISPR system. Variants from several species have been characterized. Catalyzes site-specific cleavage of double stranded DNA at sites with an TTTV (where V is A, C, or G) PAM. In some embodiments, the present disclosure provides engineered Cas12a proteins for multiplex CRISPR-based genetic modulation.

In certain embodiments, the engineered Cas12a protein is a deactivated Cas protein. As used herein, a “deactivated Cas protein” (dCas) refers to a nuclease comprising a domain that retains the ability to bind its target nucleic acid but has a diminished, or eliminated, ability to cleave a nucleic acid molecule, as compared to a control nuclease. In certain embodiments, a catalytically inactive nuclease is derived from a “wild type” Cas protein. A “wild type” nuclease refers to a naturally-occurring nuclease. A catalytically inactive Cas12a can produce a nick in the targeting DNA strand. In some embodiments, the catalytically inactive Cas12a can produce a nick in the non-targeting DNA strand. In some embodiments, the catalytically inactive Cas12a, referred to as nuclease dead Cas12a (dCas12a), lacks all DNase activity. In some aspects, the engineered Cas12a proteins are variants of nuclease dead Cas12a from Lachnospiraceae bacterium (LbdCas12a). In an exemplary embodiment, the engineered Cas12a protein is a quadruple dCas12a mutant protein having the D156R, D235R, E292R, and D350R mutations, also called the very good dCas12a, or “vgdCas12a” or “hyperdCas12a” for short. The present disclosure demonstrates the vgdCas12a in transcriptional activation of reporter genes (such as BFP or GFP), as well as endogenous genes (such as, Klf4 Sox2, and Oct4). The engineered Cas12a proteins provided herein exhibit minimal off-target effects compared to the wildtype Cas12a protein. Further, the vgdCas12a provided herein have enhanced function in gene activation, repression, and base editing. The present discourse also demonstrates that delivery of a single plasmid encoding vgdCas12a along with a poly-crRNA array simultaneously targeting endogenous Oct4, Sox2, and Klf4 loci in retina of postnatal mice drives differentiation of retinal progenitor cells.

In other aspects, the engineered Cas12a proteins are variants of nuclease active Cas12a from Lachnospiraceae bacterium (LbCas12a). The present disclosure demonstrates that the four activity-enhancing mutations, when introduced into the nuclease-active form of Cas12a, enable the resulting engineered Cas12a protein, vgCas12a (a.k.a., very good Cas12a) to have more effective gene knockout or repression activity.

In some embodiments, the engineered Cas12a proteins comprise a sequence that is at least 65%, 70%, 75%, or 80% identical to the amino acid sequence of wildtype (WT) LbdCas12a or WT nuclease active form of lbCas12a, as set forth in SEQ ID NO: 1 or 2, respectively. In some embodiments, the engineered Cas12a protein comprises one or more mutations compared to the LbdCas12a or lbCas12a nucleases. In certain embodiments, the one or more mutations are selected from the list consisting of D122R, E125R, D156R, E159R, D235R, E257R, E292R, D350R, E894R, D952R, and E981R.

In other embodiments, the engineered Cas12a protein provided herein comprise one or more mutations selected from D156R, D235R, E292R, and D350R. In certain embodiments, the engineered Cas12a protein comprises at least two, three, or four mutations.

For instance, in one exemplary embodiment, an engineered Cas12a protein provided herein comprises the mutations of D156R and E292R. In another exemplary embodiment, an engineered Cas12a protein provided herein comprises the mutations of D156R and D350R. In certain embodiment, an engineered Cas12a protein provided herein comprises the mutations of D156R, E292R, and D122R. In another embodiment, an engineered Cas12a protein provided herein comprises the mutations of D156R, E292R, and D235R. In yet another embodiment, an engineered Cas12a protein provided herein comprises the mutations of D156R, E292R, and D350R. In some specific embodiment, an engineered Cas12a protein provided herein comprises all of the four mutations of D156R, D235R, E292R, and D350R.

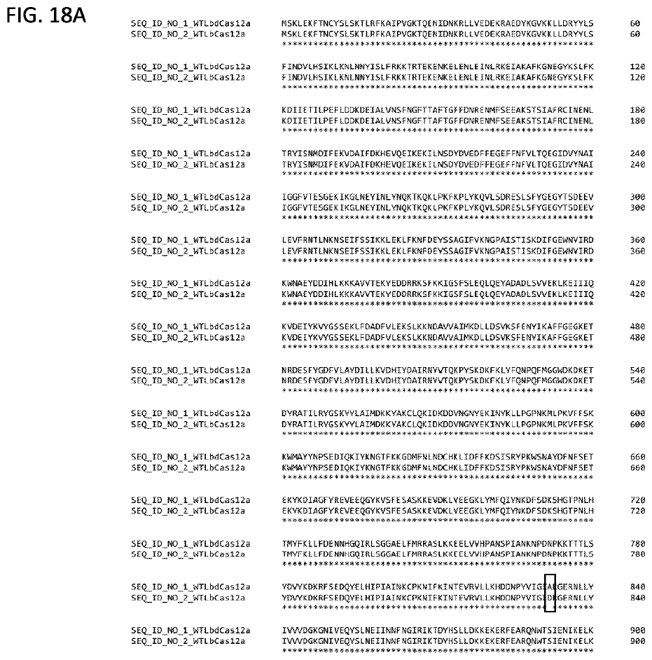

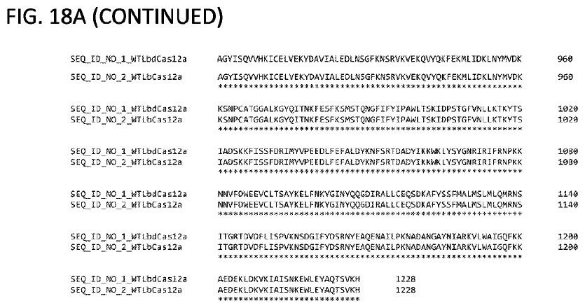

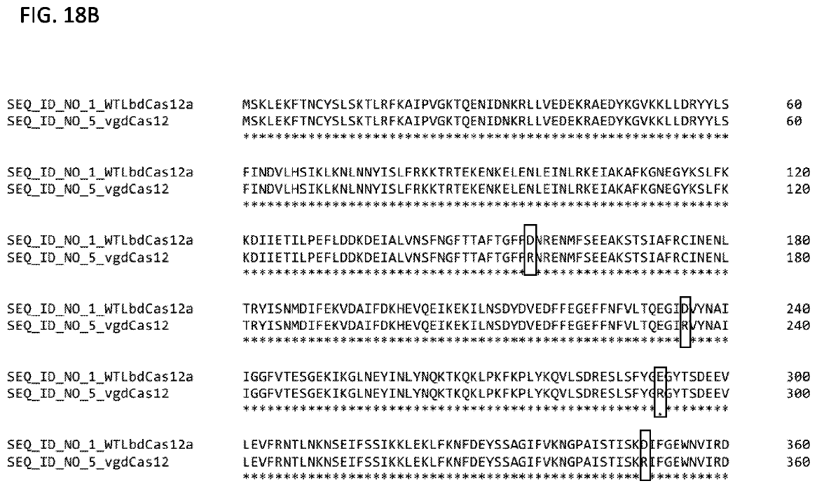

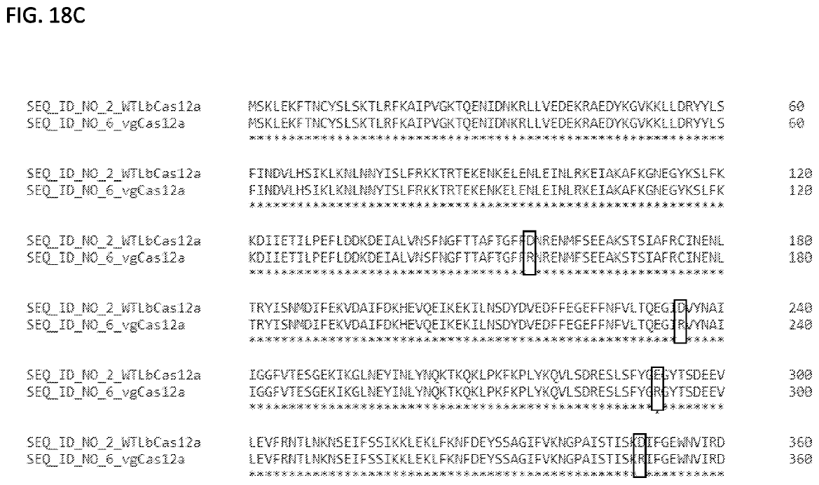

The engineered Cas12a protein provided herein can be nuclease active (i.e., having the Cas12a nuclease activity) or nuclease dead (i.e., not having the Cas12a nuclease activity). The loss of nuclease activity can be the result of mutations. For instance, a sequence alignment of a nuclease active and a nuclease dead forms of lbCas12a is illustrated in FIG. 18A, with the mutation indicated in the box.

In some exemplary embodiments, the engineered Cas12a protein provided herein comprises a sequence that is at least about 65%, 70%, 75%, 80%, 85%, 90%, 91%, 92%, 93%, 94%, 95%, 96%, 97%, 98%, 99%, or higher identity to a sequence set forth in SEQ ID NO: 5. In other exemplary embodiments, the engineered Cas12a protein provided herein comprises a sequence that is at least about 80%, 90%, or 95% identical to a sequence set forth in SEQ ID NO: 5. In one specific embodiment, the engineered Cas12a protein provided herein comprises the sequence of SEQ ID NO: 5, and the engineered Cas12a protein is a mutant nuclease dead form of LbdCas12a, also called “vgdCas12a.” The vgdCas12a protein has all of the four mutations of D156R, D235R, E292R, and D350R. A partial sequence alignment of vgdCas12a and the WT LbdCas12a is illustrated in FIG. 18B with the mutations indicated in boxes.

In some exemplary embodiments, the engineered Cas12a protein provided herein comprises a sequence that is at least about 65%, 70%, 75%, 80%, 85%, 90%, 91%, 92%, 93%, 94%, 95%, 96%, 97%, 98%, 99%, or higher identity to a sequence set forth in SEQ ID NO: 6. In other exemplary embodiments, the engineered Cas12a protein provided herein comprises a sequence that is at least about 80%, 90%, or 95% identical to a sequence set forth in SEQ ID NO: 6. In one specific embodiment, the engineered Cas12a protein provided herein comprises the sequence of SEQ ID NO: 6, and the engineered Cas12a protein is a mutant nuclease dead form of LbCas12a, also called “vgCas12a.” The vgCas12a protein has all of the four mutations of D156R, D235R, E292R, and D350R. A partial sequence alignment of vgCas12a and the WT LbCas12a is illustrated in FIG. 18C with the mutations indicated in boxes.

Exemplary sequences of the Cas12a nucleases described herein are provided in Table 1 below.

TABLE 1
Exemplary amino acid and nucleic acid sequences
of the Cas12a nucleases.

| Sequence (SEQ ID NO) | Description |
|---|---|
| MSKLEKFTNCYSLSKTLRFKAIPVGKTQENIDNKRLLVEDEKRAEDY | Amino acid |
| KGVKKLLDRYYLSFINDVLHSIKLKNLNNYISLERKKTRTEKENKEL | sequence of |
| ENLEINLRKEIAKAFKGNEGYKSLFKKDIIETILPEFLDDKDEIALV | WT |
| NSFNGFTTAFTGFFDNRENMFSEEAKSTSIAFRCINENLTRYISNMD | Lachnospiraceae |
| IFEKVDAIFDKHEVQEIKEKILNSDYDVEDFFEGEFFNFVLTQEGID | bacterium |
| VYNAIIGGFVTESGEKIKGLNEYINLYNQKTKOKLPKFKPLYKQVLS | dead Cas12a |
| DRESLSFYGEGYTSDEEVLEVERNTLNKNSEIFSSIKKLEKLFKNED | (LbdCas12a) |
| EYSSAGIFVKNGPAISTISKDIFGEWNVIRDKWNAEYDDIHLKKKAV | |
| VTEKYEDDRRKSFKKIGSFSLEQLQEYADADLSVVEKLKEIIIQKVD | |
| EIYKVYGSSEKLFDADFVLEKSLKKNDAVVAIMKDLLDSVKSFENYI | |
| KAFFGEGKETNRDESFYGDFVLAYDILLKVDHIYDAIRNYVTOKPYS | |
| KDKFKLYFQNPQFMGGWDKDKETDYRATILRYGSKYYLAIMDKKYAK | |
| CLQKIDKDDVNGNYEKINYKLLPGPNKMLPKVFFSKKWMAYYNPSED | |
| IQKIYKNGTFKKGDMFNLNDCHKLIDFFKDSISRYPKWSNAYDENFS | |
| ETEKYKDIAGFYREVEEQGYKVSFESASKKEVDKLVEEGKLYMFQIY | |
| NKDFSDKSHGTPNLHTMYFKLLFDENNHGQIRLSGGAELFMRRASLK | |
| KEELVVHPANSPIANKNPDNPKKTTTLSYDVYKDKRFSEDQYELHIP | |
| IAINKCPKNIFKINTEVRVLLKHDDNPYVIGIARGERNLLYIVVVDG | |
| KGNIVEQYSLNEIINNENGIRIKTDYHSLLDKKEKERFEARQNWTSI | |
| ENIKELKAGYISQVVHKICELVEKYDAVIALEDLNSGFKNSRVKVEK | |
| QVYQKFEKMLIDKLNYMVDKKSNPCATGGALKGYOITNKFESFKSMS | |
| TONGFIFYIPAWLTSKIDPSTGFVNLLKTKYTSIADSKKFISSEDRI | |
| MYVPEEDLFEFALDYKNFSRTDADYIKKWKLYSYGNRIRIFRNPKKN | |
| NVFDWEEVCLTSAYKELENKYGINYQQGDIRALLCEQSDKAFYSSFM | |
| ALMSLMLQMRNSITGRTDVDFLISPVKNSDGIFYDSRNYEAQENAIL | |
| PKNADANGAYNIARKVLWAIGQFKKAEDEKLDKVKIAISNKEWLEYA | |
| QTSVKH (SEQ ID NO: 1) | |
| MSKLEKFTNCYSLSKTLRFKAIPVGKTQENIDNKRLLVEDEKRAEDY | Amino acid |
| KGVKKLLDRYYLSFINDVLHSIKLKNLNNYISLERKKTRTEKENKEL | sequence of |
| ENLEINLRKEIAKAFKGNEGYKSLFKKDIIETILPEFLDDKDEIALV | WT nuclease |
| NSFNGFTTAFTGFFDNRENMFSEEAKSTSIAFRCINENLTRYISNMD | active form |
| IFEKVDAIFDKHEVQEIKEKILNSDYDVEDFFEGEFFNFVLTQEGID | of lbCas12a |
| VYNAIIGGFVTESGEKIKGLNEYINLYNQKTKQKLPKFKPLYKQVLS | |
| DRESLSFYGEGYTSDEEVLEVFRNTLNKNSEIFSSIKKLEKLFKNED | |
| EYSSAGIFVKNGPAISTISKDIFGEWNVIRDKWNAEYDDIHLKKKAV | |
| VTEKYEDDRRKSFKKIGSFSLEQLQEYADADLSVVEKLKEIIIQKVD | |
| EIYKVYGSSEKLFDADFVLEKSLKKNDAVVAIMKDLLDSVKSFENYI | |
| KAFFGEGKETNRDESFYGDFVLAYDILLKVDHIYDAIRNYVTOKPYS | |
| KDKFKLYFQNPQFMGGWDKDKETDYRATILRYGSKYYLAIMDKKYAK | |
| CLQKIDKDDVNGNYEKINYKLLPGPNKMLPKVFFSKKWMAYYNPSED | |
| IQKIYKNGTFKKGDMENLNDCHKLIDFFKDSISRYPKWSNAYDENFS | |
| ETEKYKDIAGFYREVEEQGYKVSFESASKKEVDKLVEEGKLYMFQIY | |
| NKDFSDKSHGTPNLHTMYFKLLFDENNHGQIRLSGGAELFMRRASLK | |
| KEELVVHPANSPIANKNPDNPKKTTTLSYDVYKDKRFSEDQYELHIP | |
| IAINKCPKNIFKINTEVRVLLKHDDNPYVIGIDRGERNLLYIVVVDG | |
| KGNIVEQYSLNEIINNFNGIRIKTDYHSLLDKKEKERFEARQNWTSI | |
| ENIKELKAGYISQVVHKICELVEKYDAVIALEDLNSGFKNSRVKVEK | |
| QVYQKFEKMLIDKLNYMVDKKSNPCATGGALKGYQITNKFESFKSMS | |
| TONGFIFYIPAWLTSKIDPSTGFVNLLKTKYTSIADSKKFISSFDRI | |
| MYVPEEDLFEFALDYKNFSRTDADYIKKWKLYSYGNRIRIFRNPKKN | |
| NVFDWEEVCLTSAYKELENKYGINYQQGDIRALLCEQSDKAFYSSFM | |
| ALMSLMLOMRNSITGRTDVDFLISPVKNSDGIFYDSRNYEAQENAIL | |
| PKNADANGAYNIARKVLWAIGQFKKAEDEKLDKVKIAISNKEWLEYA | |
| QTSVKH (SEQ ID NO: 2) | |
| ATGAGCAAGCTGGAGAAGTTTACaaactgctactccctgtctaagac | Nucleic acid |
| cctgaggttcaaggccatccctgtgggcaagacccaggagaacatcg | sequence of |
| acaataagcggctgctggtggaggacgagaagagagccgaggattat | WT LbdCas12a |
| aagggcgtgaagaagctgctggatcgctactatctgtcttttatcaa | |
| cgacgtgctgcacagcatcaagctgaagaatctgaacaattacatca | |
| gcctgttccggaagaaaaccagaaccgagaaggagaataaggagctg | |
| gagaacctggagatcaatctgcggaaggagatcgccaaggccttcaa | |
| gggcaacgagggctacaagtccctgtttaagaaggatatcatcgaga | |
| caatcctgccagagttcctggacgataaggacgagatcgccctggtg | |
| aacagcttcaatggctttaccacagccttcaccggcttctttgataa | |
| cagagagaatatgttttccgaggaggccaagagcacatccatcgcct | |
| tcaggtgtatcaacgagaatctgacccgctacatctctaatatggac | |
| atcttcgagaaggtggacgccatctttgataagcacgaggtgcagga | |
| gatcaaggagaagatcctgaacagcgactatgatgtggaggatttct | |
| ttgagggcgagttctttaactttgtgctgacacaggagggcatcgac | |
| gtgtataacgccatcatcggcggcttcgtgaccgagagcggcgagaa | |
| gatcaagggcctgaacGAgtacatcaacctgtataatcagaaaacca | |
| agcagaagctgcctaagtttaagccactgtataagcaggtgctgagc | |
| gatcgggagtctctgagcttctacggcgagggctatacatccgatga | |
| ggaggtgctggaggtgtttagaaacaccctgaacaagaacagcgaga | |
| tcttcagctccatcaagaagctggagaagctgttcaagaattttgac | |
| gagtactctagcgccggcatctttgtgaagaacggccccgccatcag | |
| cacaatctccaaggatatcttcggcgagtggaacgtgatccgggaca | |
| agtggaatgccgagtatgacgatatccacctgaagaagaaggccgtg | |
| gtgaccgagaagtacgaggacgatcggagaaagtccttcaagaagat | |
| cggctccttttctctggagcagctgcaggagtacgccgacgccgatc | |
| tgtctgtggtggagaagctgaaggagatcatcatccagaaggtggat | |
| gagatctacaaggtgtatggctcctctgagaagctgttcgacgccga | |
| ttttgtgctggagaagagcctgaagaagaacgacgccgtggtggcca | |
| tcatgaaggacctgctggattctgtgaagagcttcgagaattacatc | |
| aaggccttctttggcgagggcaaggagacaaacagggacgagtcctt | |
| ctatggcgattttgtgctggcctacgacatcctgctgaaggtggacc | |
| acatctacgatgccatccgcaattatgtgacccagaagccctactct | |
| aaggataagttcaagctgtattttcagaaccctcagttcatgggcgg | |
| ctgggacaaggataaggagacagactatcgggccaccatcctgagat | |
| acggctccaagtactatctggccatcatggataagaagtacgccaag | |
| tgcctgcagaagatcgacaaggacgatgtgaacggcaattacgagaa | |
| gatcaactataagctgctgcccggccctaataagatgctgccaaagg | |
| tgttcttttctaagaagtggatggcctactataaccccagcgaggac | |
| atccagaagatctacaagaatggcacattcaagaagggcgatatgtt | |
| taacctgaatgactgtcacaagctgatcgacttctttaaggatagca | |
| tctcccggtatccaaagtggtccaatgcctacgatttcaacttttct | |
| gagacagagaagtataaggacatcgccggcttttacagagaggtgga | |
| ggagcagggctataaggtgagcttcgagtctgccagcaagaaggagg | |
| tggataagctggtggaggagggcaagctgtatatgttccagatctat | |
| aacaaggacttttccgataagtctcacggcacacccaatctgcacac | |
| catgtacttcaagctgctgtttgacgagaacaatcacggacagatca | |
| ggctgagcggaggagcagagctgttcatgaggcgcgcctccctgaag | |
| aaggaggagctggtggtgcacccagccaactcccctatcgccaacaa | |
| gaatccagataatcccaagaaaaccacaaccctgtcctacgacgtgt | |
| ataaggataagaggttttctgaggaccagtacgagctgcacatccca | |
| atcgccatcaataagtgccccaagaacatcttcaagatcaatacaga | |
| ggtgcgcgtgctgctgaagcacgacgataacccctatgtgatcggca | |
| tcgccaggggcgagcgcaatctgctgtatatcgtggtggtggacggc | |
| aagggcaacatcgtggagcagtattccctgaacgagatcatcaacaa | |
| cttcaacggcatcaggatcaagacagattaccactctctgctggaca | |
| agaaggagaaggagaggttcgaggcccgccagaactggacctccatc | |
| gagaatatcaaggagctgaaggccggctatatctctcaggtggtgca | |
| caagatctgcgagctggtggagaagtacgatgccgtgatcgccctgg | |
| aggacctgaactctggctttaagaatagccgcgtgaaggtggagaag | |
| caggtgtatcagaagttcgagaagatgctgatcgataagctgaacta | |
| catggtggacaagaagtctaatccttgtgcaacaggcggcgccctga | |
| agggctatcagatcaccaataagttcgagagctttaagtccatgtct | |
| acccagaacggcttcatcttttacatccctgcctggctgacatccaa | |
| gatcgatccatctaccggctttgtgaacctgctgaaaaccaagtata | |
| ccagcatcgccgattccaagaagttcatcagctcctttgacaggatc | |
| atgtacgtgcccgaggaggatctgttcgagtttgccctggactataa | |
| gaacttctctcgcacagacgccgattacatcaagaagtggaagctgt | |
| actcctacggcaaccggatcagaatcttccggaatcctaagaagaac | |
| aacgtgttcgactgggaggaggtgtgcctgaccagcgcctataagga | |
| gctgttcaacaagtacggcatcaattatcagcagggcgatatcagag | |
| ccctgctgtgcgagcagtccgacaaggccttctactctagctttatg | |
| gccctgatgagcctgatgctgcagatgcggaacagcatcacaggccg | |
| caccgacgtggattttctgatcagccctgtgaagaactccgacggca | |
| tcttctacgatagccggaactatgaggcccaggagaatgccatcctg | |
| ccaaagaacgccgacgccaatggcgcctataacatcgccagaaaggt | |
| gctgtgggccatcggccagttcaagaaggccgaggacgagaagctgg | |
| ataaggtgaagatcgccatctctaacaaggagtggctggagtacgcc | |
| cagaccagcgtgaagcac (SEQ ID NO: 3) | |
| atgagcaagctggagaagtttacaaactgctactccctgtctaagac | Nucleic acid |
| cctgaggttcaaggccatccctgtgggcaagacccaggagaacatcg | sequence of |
| acaataagcggctgctggtggaggacgagaagagagccgaggattat | WT nuclease |
| aagggcgtgaagaagctgctggatcgctactatctgtcttttatcaa | active form |
| cgacgtgctgcacagcatcaagctgaagaatctgaacaattacatca | of lbCas12a |
| gcctgttccggaagaaaaccagaaccgagaaggagaataaggagctg | |
| gagaacctggagatcaatctgcggaaggagatcgccaaggccttcaa | |
| gggcaacgagggctacaagtccctgtttaagaaggatatcatcgaga | |
| caatcctgccagagttcctggacgataaggacgagatcgccctggtg | |
| aacagcttcaatggctttaccacagccttcaccggcttctttgataa | |
| cagagagaatatgttttccgaggaggccaagagcacatccatcgcct | |
| tcaggtgtatcaacgagaatctgacccgctacatctctaatatggac | |
| atcttcgagaaggtggacgccatctttgataagcacgaggtgcagga | |
| gatcaaggagaagatcctgaacagcgactatgatgtggaggatttct | |
| ttgagggcgagttctttaactttgtgctgacacaggagggcatcgac | |
| gtgtataacgccatcatcggcggcttcgtgaccgagagcggcgagaa | |
| gatcaagggcctgaacgagtacatcaacctgtataatcagaaaacca | |
| agcagaagctgcctaagtttaagccactgtataagcaggtgctgagc | |
| gatcgggagtctctgagcttctacggcgagggctatacatccgatga | |
| ggaggtgctggaggtgtttagaaacaccctgaacaagaacagcgaga | |
| tcttcagctccatcaagaagctggagaagctgttcaagaattttgac | |
| gagtactctagcgccggcatctttgtgaagaacggccccgccatcag | |
| cacaatctccaaggatatcttcggcgagtggaacgtgatccgggaca | |
| agtggaatgccgagtatgacgatatccacctgaagaagaaggccgtg | |
| gtgaccgagaagtacgaggacgatcggagaaagtccttcaagaagat | |
| cggctccttttctctggagcagctgcaggagtacgccgacgccgato | |
| tgtctgtggtggagaagctgaaggagatcatcatccagaaggtggat | |
| gagatctacaaggtgtatggctcctctgagaagctgttcgacgccga | |
| ttttgtgctggagaagagcctgaagaagaacgacgccgtggtggcca | |
| tcatgaaggacctgctggattctgtgaagagcttcgagaattacatc | |
| aaggccttctttggcgagggcaaggagacaaacagggacgagtcctt | |
| ctatggcgattttgtgctggcctacgacatcctgctgaaggtggacc | |
| acatctacgatgccatccgcaattatgtgacccagaagccctactct | |
| aaggataagttcaagctgtattttcagaaccctcagttcatgggcgg | |
| ctgggacaaggataaggagacagactatcgggccaccatcctgagat | |
| acggctccaagtactatctggccatcatggataagaagtacgccaag | |
| tgcctgcagaagatcgacaaggacgatgtgaacggcaattacgagaa | |
| gatcaactataagctgctgcccggccctaataagatgctgccaaagg | |
| tgttcttttctaagaagtggatggcctactataaccccagcgaggac | |
| atccagaagatctacaagaatggcacattcaagaagggcgatatgtt | |
| taacctgaatgactgtcacaagctgatcgacttctttaaggatagca | |
| tctcccggtatccaaagtggtccaatgcctacgatttcaacttttct | |
| gagacagagaagtataaggacatcgccggcttttacagagaggtgga | |
| ggagcagggctataaggtgagcttcgagtctgccagcaagaaggagg | |
| tggataagctggtggaggagggcaagctgtatatgttccagatctat | |
| aacaaggacttttccgataagtctcacggcacacccaatctgcacac | |
| catgtacttcaagctgctgtttgacgagaacaatcacggacagatca | |
| ggctgagcggaggagcagagctgttcatgaggcgcgcctccctgaag | |
| aaggaggagctggtggtgcacccagccaactcccctatcgccaacaa | |
| gaatccagataatcccaagaaaaccacaaccctgtcctacgacgtgt | |
| ataaggataagaggttttctgaggaccagtacgagctgcacatccca | |
| atcgccatcaataagtgccccaagaacatcttcaagatcaatacaga | |
| ggtgcgcgtgctgctgaagcacgacgataacccctatgtgatcggca | |
| tcGATaggggcgagcgcaatctgctgtatatcgtggtggtggacggc | |
| aagggcaacatcgtggagcagtattccctgaacgagatcatcaacaa | |
| cttcaacggcatcaggatcaagacagattaccactctctgctggaca | |
| agaaggagaaggagaggttcgaggcccgccagaactggacctccatc | |
| gagaatatcaaggagctgaaggccggctatatctctcaggtggtgca | |
| caagatctgcgagctggtggagaagtacgatgccgtgatcgccctgg | |
| aggacctgaactctggctttaagaatagccgcgtgaaggtggagaag | |
| caggtgtatcagaagttcgagaagatgctgatcgataagctgaacta | |
| catggtggacaagaagtctaatccttgtgcaacaggcggcgccctga | |
| agggctatcagatcaccaataagttcgagagctttaagtccatgtct | |
| acccagaacggcttcatcttttacatccctgcctggctgacatccaa | |
| gatcgatccatctaccggctttgtgaacctgctgaaaaccaagtata | |
| ccagcatcgccgattccaagaagttcatcagctcctttgacaggatc | |
| atgtacgtgcccgaggaggatctgttcgagtttgccctggactataa | |
| gaacttctctcgcacagacgccgattacatcaagaagtggaagctgt | |
| actcctacggcaaccggatcagaatcttccggaatcctaagaagaac | |
| aacgtgttcgactgggaggaggtgtgcctgaccagcgcctataagga | |
| gctgttcaacaagtacggcatcaattatcagcagggcgatatcagag | |
| ccctgctgtgcgagcagtccgacaaggccttctactctagctttatg | |
| gccctgatgagcctgatgctgcagatgcggaacagcatcacaggccg | |
| caccgacgtggattttctgatcagccctgtgaagaactccgacggca | |
| tcttctacgatagccggaactatgaggcccaggagaatgccatcctg | |
| ccaaagaacgccgacgccaatggcgcctataacatcgccagaaaggt | |
| gctgtgggccatcggccagttcaagaaggccgaggacgagaagctgg | |
| ataaggtgaagatcgccatctctaacaaggagtggctggagtacgcc | |
| cagaccagcgtgaagcac (SEQ ID NO: 4) | |
| MSKLEKFTNCYSLSKTLRFKAIPVGKTQENIDNKRLLVEDEKRAEDY | Amino acid |
| KGVKKLLDRYYLSFINDVLHSIKLKNLNNYISLERKKTRTEKENKEL | sequence of |
| ENLEINLRKEIAKAFKGNEGYKSLEKKDIIETILPEFLDDKDEIALV | mutant |
| NSFNGFTTAFTGFERNRENMESEEAKSTSIAFRCINENLTRYISNMD | nuclease |
| IFEKVDAIFDKHEVQEIKEKILNSDYDVEDFFEGEFFNFVLTQEGIR | dead form of |
| VYNAIIGGFVTESGEKIKGLNEYINLYNQKTKQKLPKFKPLYKQVLS | LbdCas12a |
| DRESLSFYGRGYTSDEEVLEVERNTLNKNSEIFSSIKKLEKLFKNED | (vgdCas12a) |
| EYSSAGIFVKNGPAISTISKRIFGEWNVIRDKWNAEYDDIHLKKKAV | |
| VTEKYEDDRRKSFKKIGSFSLEQLQEYADADLSVVEKLKEIIIQKVD | |
| EIYKVYGSSEKLFDADFVLEKSLKKNDAVVAIMKDLLDSVKSFENYI | |
| KAFFGEGKETNRDESFYGDFVLAYDILLKVDHIYDAIRNYVTOKPYS | |
| KDKFKLYFQNPQFMGGWDKDKETDYRATILRYGSKYYLAIMDKKYAK | |
| CLQKIDKDDVNGNYEKINYKLLPGPNKMLPKVFFSKKWMAYYNPSED | |
| IQKIYKNGTFKKGDMENLNDCHKLIDFFKDSISRYPKWSNAYDENFS | |
| ETEKYKDIAGFYREVEEQGYKVSFESASKKEVDKLVEEGKLYMFQIY | |
| NKDFSDKSHGTPNLHTMYFKLLFDENNHGQIRLSGGAELFMRRASLK | |
| KEELVVHPANSPIANKNPDNPKKTTTLSYDVYKDKRFSEDQYELHIP | |
| IAINKCPKNIFKINTEVRVLLKHDDNPYVIGIARGERNLLYIVVVDG | |
| KGNIVEQYSLNEIINNENGIRIKTDYHSLLDKKEKERFEARONWTSI | |
| ENIKELKAGYISQVVHKICELVEKYDAVIALEDLNSGFKNSRVKVEK | |
| QVYQKFEKMLIDKLNYMVDKKSNPCATGGALKGYQITNKFESFKSMS | |
| TONGFIFYIPAWLTSKIDPSTGFVNLLKTKYTSIADSKKFISSFDRI | |
| MYVPEEDLFEFALDYKNFSRTDADYIKKWKLYSYGNRIRIFRNPKKN | |
| NVFDWEEVCLTSAYKELENKYGINYQQGDIRALLCEQSDKAFYSSEM | |
| ALMSLMLQMRNSITGRTDVDFLISPVKNSDGIFYDSRNYEAQENAIL | |
| PKNADANGAYNIARKVLWAIGQFKKAEDEKLDKVKIAISNKEWLEYA | |
| QTSVKH (SEQ ID NO: 5) | |
| MSKLEKFTNCYSLSKTLRFKAIPVGKTQENIDNKRLLVEDEKRAEDY | Amino acid |
| KGVKKLLDRYYLSFINDVLHSIKLKNLNNYISLFRKKTRTEKENKEL | sequence of |
| ENLEINLRKEIAKAFKGNEGYKSLFKKDIIETILPEFLDDKDEIALV | mutant |
| NSFNGFTTAFTGFFRNRENMFSEEAKSTSIAFRCINENLTRYISNMD | nuclease |
| IFEKVDAIFDKHEVQEIKEKILNSDYDVEDFFEGEFFNFVLTQEGIR | active form |
| VYNAIIGGFVTESGEKIKGLNEYINLYNOKTKOKLPKFKPLYKQVLS | of lbCas12a |
| DRESLSFYGRGYTSDEEVLEVERNTLNKNSEIFSSIKKLEKLFKNED | (vgCas12a) |
| EYSSAGIFVKNGPAISTISKRIFGEWNVIRDKWNAEYDDIHLKKKAV | |
| VTEKYEDDRRKSFKKIGSESLEQLQEYADADLSVVEKLKEIIIQKVD | |
| EIYKVYGSSEKLFDADFVLEKSLKKNDAVVAIMKDLLDSVKSFENYI | |
| KAFFGEGKETNRDESFYGDFVLAYDILLKVDHIYDAIRNYVTOKPYS | |
| KDKFKLYFQNPQFMGGWDKDKETDYRATILRYGSKYYLAIMDKKYAK | |
| CLQKIDKDDVNGNYEKINYKLLPGPNKMLPKVFFSKKWMAYYNPSED | |
| IQKIYKNGTFKKGDMENLNDCHKLIDFFKDSISRYPKWSNAYDENES | |
| ETEKYKDIAGFYREVEEQGYKVSFESASKKEVDKLVEEGKLYMFQIY | |
| NKDFSDKSHGTPNLHTMYFKLLFDENNHGQIRLSGGAELEMRRASLK | |
| KEELVVHPANSPIANKNPDNPKKTTTLSYDVYKDKRFSEDQYELHIP | |
| IAINKCPKNIFKINTEVRVLLKHDDNPYVIGIDRGERNLLYIVVVDG | |
| KGNIVEQYSLNEIINNENGIRIKTDYHSLLDKKEKERFEARQNWTSI | |
| ENIKELKAGYISQVVHKICELVEKYDAVIALEDLNSGFKNSRVKVEK | |
| QVYQKFEKMLIDKLNYMVDKKSNPCATGGALKGYQITNKFESFKSMS | |
| TONGFIFYIPAWLISKIDPSTGFVNLLKTKYTSIADSKKFISSEDRI | |
| MYVPEEDLFEFALDYKNFSRTDADYIKKWKLYSYGNRIRIFRNPKKN | |
| NVFDWEEVCLTSAYKELFNKYGINYQQGDIRALLCEQSDKAFYSSEM | |
| ALMSLMLQMRNSITGRTDVDFLISPVKNSDGIFYDSRNYEAQENAIL | |
| PKNADANGAYNIARKVLWAIGQFKKAEDEKLDKVKIAISNKEWLEYA | |
| QTSVKH (SEQ ID NO: 6) | |
| ATGAGCAAGCTGGAGAAGTTTACaaactgctactccctgtctaagac | Nucleic acid |
| cctgaggttcaaggccatccctgtgggcaagacccaggagaacatcg | sequence of |
| acaataagcggctgctggtggaggacgagaagagagccgaggattat | mutant |
| aagggcgtgaagaagctgctggatcgctactatctgtcttttatcaa | LbdCas12a |
| cgacgtgctgcacagcatcaagctgaagaatctgaacaattacatca | (vgdCas12a) |
| gcctgttccggaagaaaaccagaaccgagaaggagaataaggagctg | |
| gagaacctggagatcaatctgcggaaggagatcgccaaggccttcaa | |
| gggcaacgagggctacaagtccctgtttaagaaggatatcatcgaga | |
| caatcctgccagagttcctggacgataaggacgagatcgccctggtg | |
| aacagcttcaatggctttaccacagccttcaccggcttctttcgtaa | |
| cagagagaatatgttttccgaggaggccaagagcacatccatcgcct | |
| tcaggtgtatcaacgagaatctgacccgctacatctctaatatggac | |
| atcttcgagaaggtggacgccatctttgataagcacgaggtgcagga | |
| gatcaaggagaagatcctgaacagcgactatgatgtggaggatttct | |
| ttgagggcgagttctttaactttgtgctgacacaggagggcatccgc | |
| gtgtataacgccatcatcggcggcttcgtgaccgagagcggcgagaa | |
| gatcaagggcctgaacGAgtacatcaacctgtataatcagaaaacca | |
| agcagaagctgcctaagtttaagccactgtataagcaggtgctgagc | |
| gatcgggagtctctgagcttctacggcaggggctatacatccgatga | |
| ggaggtgctggaggtgtttagaaacaccctgaacaagaacagcgaga | |
| tcttcagctccatcaagaagctggagaagctgttcaagaattttgac | |
| gagtactctagcgccggcatctttgtgaagaacggccccgccatcag | |
| cacaatctccaagcgtatcttcggcgagtggaacgtgatccgggaca | |
| agtggaatgccgagtatgacgatatccacctgaagaagaaggccgtg | |
| gtgaccgagaagtacgaggacgatcggagaaagtccttcaagaagat | |
| cggctccttttctctggagcagctgcaggagtacgccgacgccgatc | |
| tgtctgtggtggagaagctgaaggagatcatcatccagaaggtggat | |
| gagatctacaaggtgtatggctcctctgagaagctgttcgacgccga | |
| ttttgtgctggagaagagcctgaagaagaacgacgccgtggtggcca | |
| tcatgaaggacctgctggattctgtgaagagcttcgagaattacatc | |
| aaggccttctttggcgagggcaaggagacaaacagggacgagtcctt | |
| ctatggcgattttgtgctggcctacgacatcctgctgaaggtggacc | |
| acatctacgatgccatccgcaattatgtgacccagaagccctactct | |
| aaggataagttcaagctgtattttcagaaccctcagttcatgggcgg | |
| ctgggacaaggataaggagacagactatcgggccaccatcctgagat | |
| acggctccaagtactatctggccatcatggataagaagtacgccaag | |
| tgcctgcagaagatcgacaaggacgatgtgaacggcaattacgagaa | |
| gatcaactataagctgctgcccggccctaataagatgctgccaaagg | |
| tgttcttttctaagaagtggatggcctactataaccccagcgaggac | |
| atccagaagatctacaagaatggcacattcaagaagggcgatatgtt | |
| taacctgaatgactgtcacaagctgatcgacttctttaaggatagca | |
| tctcccggtatccaaagtggtccaatgcctacgatttcaacttttct | |
| gagacagagaagtataaggacatcgccggcttttacagagaggtgga | |
| ggagcagggctataaggtgagcttcgagtctgccagcaagaaggagg | |
| tggataagctggtggaggagggcaagctgtatatgttccagatctat | |
| aacaaggacttttccgataagtctcacggcacacccaatctgcacac | |
| catgtacttcaagctgctgtttgacgagaacaatcacggacagatca | |
| ggctgagcggaggagcagagctgttcatgaggcgcgcctccctgaag | |
| aaggaggagctggtggtgcacccagccaactcccctatcgccaacaa | |
| gaatccagataatcccaagaaaaccacaaccctgtcctacgacgtgt | |
| ataaggataagaggttttctgaggaccagtacgagctgcacatccca | |
| atcgccatcaataagtgccccaagaacatcttcaagatcaatacaga | |
| ggtgcgcgtgctgctgaagcacgacgataacccctatgtgatcggca | |
| tcgccaggggcgagcgcaatctgctgtatatcgtggtggtggacggc | |
| aagggcaacatcgtggagcagtattccctgaacgagatcatcaacaa | |
| cttcaacggcatcaggatcaagacagattaccactctctgctggaca | |
| agaaggagaaggagaggttcgaggcccgccagaactggacctccatc | |
| gagaatatcaaggagctgaaggccggctatatctctcaggtggtgca | |
| caagatctgcgagctggtggagaagtacgatgccgtgatcgccctgg | |
| aggacctgaactctggctttaagaatagccgcgtgaaggtggagaag | |
| caggtgtatcagaagttcgagaagatgctgatcgataagctgaacta | |
| catggtggacaagaagtctaatccttgtgcaacaggcggcgccctga | |
| agggctatcagatcaccaataagttcgagagctttaagtccatgtct | |
| acccagaacggcttcatcttttacatccctgcctggctgacatccaa | |
| gatcgatccatctaccggctttgtgaacctgctgaaaaccaagtata | |
| ccagcatcgccgattccaagaagttcatcagctcctttgacaggatc | |
| atgtacgtgcccgaggaggatctgttcgagtttgccctggactataa | |
| gaacttctctcgcacagacgccgattacatcaagaagtggaagctgt | |
| actcctacggcaaccggatcagaatcttccggaatcctaagaagaac | |
| aacgtgttcgactgggaggaggtgtgcctgaccagcgcctataagga | |
| gctgttcaacaagtacggcatcaattatcagcagggcgatatcagag | |
| ccctgctgtgcgagcagtccgacaaggccttctactctagctttatg | |
| gccctgatgagcctgatgctgcagatgcggaacagcatcacaggccg | |
| caccgacgtggattttctgatcagccctgtgaagaactccgacggca | |
| tcttctacgatagccggaactatgaggcccaggagaatgccatcctg | |
| ccaaagaacgccgacgccaatggcgcctataacatcgccagaaaggt | |
| gctgtgggccatcggccagttcaagaaggccgaggacgagaagctgg | |
| ataaggtgaagatcgccatctctaacaaggagtggctggagtacgcc | |
| cagaccagcgtgaagcac (SEQ ID NO: 7) | |
| ATGAGCAAGCTGGAGAAGTTTACaaactgctactccctgtctaagac | Nucleic acid |
| cctgaggttcaaggccatccctgtgggcaagacccaggagaacatcg | sequence of |
| acaataagcggctgctggtggaggacgagaagagagccgaggattat | mutant |
| aagggcgtgaagaagctgctggatcgctactatctgtcttttatcaa | nuclease |
| cgacgtgctgcacagcatcaagctgaagaatctgaacaattacatca | active form |
| gcctgttccggaagaaaaccagaaccgagaaggagaataaggagctg | of lbCas12a |
| gagaacctggagatcaatctgcggaaggagatcgccaaggccttcaa | (vgCas12a) |
| gggcaacgagggctacaagtccctgtttaagaaggatatcatcgaga | |
| caatcctgccagagttcctggacgataaggacgagatcgccctggtg | |
| aacagcttcaatggctttaccacagccttcaccggcttctttcgtaa | |
| cagagagaatatgttttccgaggaggccaagagcacatccatcgcct | |
| tcaggtgtatcaacgagaatctgacccgctacatctctaatatggac | |
| atcttcgagaaggtggacgccatctttgataagcacgaggtgcagga | |
| gatcaaggagaagatcctgaacagcgactatgatgtggaggatttct | |
| ttgagggcgagttctttaactttgtgctgacacaggagggcatccgc | |
| gtgtataacgccatcatcggcggcttcgtgaccgagagcggcgagaa | |
| gatcaagggcctgaacGAgtacatcaacctgtataatcagaaaacca | |
| agcagaagctgcctaagtttaagccactgtataagcaggtgctgagc | |
| gatcgggagtctctgagcttctacggcaggggctatacatccgatga | |
| ggaggtgctggaggtgtttagaaacaccctgaacaagaacagcgaga | |
| tcttcagctccatcaagaagctggagaagctgttcaagaattttgac | |
| gagtactctagcgccggcatctttgtgaagaacggccccgccatcag | |
| cacaatctccaagcgtatcttcggcgagtggaacgtgatccgggaca | |
| agtggaatgccgagtatgacgatatccacctgaagaagaaggccgtg | |
| gtgaccgagaagtacgaggacgatcggagaaagtccttcaagaagat | |
| cggctccttttctctggagcagctgcaggagtacgccgacgccgatc | |
| tgtctgtggtggagaagctgaaggagatcatcatccagaaggtggat | |
| gagatctacaaggtgtatggctcctctgagaagctgttcgacgccga | |
| ttttgtgctggagaagagcctgaagaagaacgacgccgtggtggcca | |
| tcatgaaggacctgctggattctgtgaagagcttcgagaattacatc | |
| aaggccttctttggcgagggcaaggagacaaacagggacgagtcctt | |
| ctatggcgattttgtgctggcctacgacatcctgctgaaggtggacc | |
| acatctacgatgccatccgcaattatgtgacccagaagccctactct | |
| aaggataagttcaagctgtattttcagaaccctcagttcatgggcgg | |
| ctgggacaaggataaggagacagactatcgggccaccatcctgagat | |
| acggctccaagtactatctggccatcatggataagaagtacgccaag | |
| tgcctgcagaagatcgacaaggacgatgtgaacggcaattacgagaa | |
| gatcaactataagctgctgcccggccctaataagatgctgccaaagg | |
| tgttcttttctaagaagtggatggcctactataaccccagcgaggac | |
| atccagaagatctacaagaatggcacattcaagaagggcgatatgtt | |
| taacctgaatgactgtcacaagctgatcgacttctttaaggatagca | |
| tctcccggtatccaaagtggtccaatgcctacgatttcaacttttct | |
| gagacagagaagtataaggacatcgccggcttttacagagaggtgga | |
| ggagcagggctataaggtgagcttcgagtctgccagcaagaaggagg | |
| tggataagctggtggaggagggcaagctgtatatgttccagatctat | |
| aacaaggacttttccgataagtctcacggcacacccaatctgcacac | |
| catgtacttcaagctgctgtttgacgagaacaatcacggacagatca | |
| ggctgagcggaggagcagagctgttcatgaggcgcgcctccctgaag | |
| aaggaggagctggtggtgcacccagccaactcccctatcgccaacaa | |
| gaatccagataatcccaagaaaaccacaaccctgtcctacgacgtgt | |
| ataaggataagaggttttctgaggaccagtacgagctgcacatccca | |
| atcgccatcaataagtgccccaagaacatcttcaagatcaatacaga | |
| ggtgcgcgtgctgctgaagcacgacgataacccctatgtgatcggca | |
| tcgataggggcgagcgcaatctgctgtatatcgtggtggtggacggc | |
| aagggcaacatcgtggagcagtattccctgaacgagatcatcaacaa | |
| cttcaacggcatcaggatcaagacagattaccactctctgctggaca | |
| agaaggagaaggagaggttcgaggcccgccagaactggacctccatc | |
| gagaatatcaaggagctgaaggccggctatatctctcaggtggtgca | |
| caagatctgcgagctggtggagaagtacgatgccgtgatcgccctgg | |
| aggacctgaactctggctttaagaatagccgcgtgaaggtggagaag | |
| caggtgtatcagaagttcgagaagatgctgatcgataagctgaacta | |
| catggtggacaagaagtctaatccttgtgcaacaggcggcgccctga | |
| agggctatcagatcaccaataagttcgagagctttaagtccatgtct | |
| acccagaacggcttcatcttttacatccctgcctggctgacatccaa | |
| gatcgatccatctaccggctttgtgaacctgctgaaaaccaagtata | |
| ccagcatcgccgattccaagaagttcatcagctcctttgacaggatc | |
| atgtacgtgcccgaggaggatctgttcgagtttgccctggactataa | |
| gaacttctctcgcacagacgccgattacatcaagaagtggaagctgt | |
| actcctacggcaaccggatcagaatcttccggaatcctaagaagaac | |
| aacgtgttcgactgggaggaggtgtgcctgaccagcgcctataagga | |
| gctgttcaacaagtacggcatcaattatcagcagggcgatatcagag | |
| ccctgctgtgcgagcagtccgacaaggccttctactctagctttatg | |
| gccctgatgagcctgatgctgcagatgcggaacagcatcacaggccg | |
| caccgacgtggattttctgatcagccctgtgaagaactccgacggca | |
| tcttctacgatagccggaactatgaggcccaggagaatgccatcctg | |
| ccaaagaacgccgacgccaatggcgcctataacatcgccagaaaggt | |
| gctgtgggccatcggccagttcaagaaggccgaggacgagaagctgg | |
| ataaggtgaagatcgccatctctaacaaggagtggctggagtacgcc | |
| cagaccagcgtgaagcac (SEQ ID NO: 8) | |

The engineered Cas12a proteins provided herein exhibit improved activities compared to the corresponding WT Cas12a protein, i.e., the nuclease active form or the nuclease dead form, respectively.

For instance, in some embodiments, the present disclosure demonstrates that the engineered Cas12a protein provided herein exhibit improved activation compared to the WT Cas12a protein, as shown in Example 3. In some embodiments, the engineered Cas12a protein provided herein exhibits improved repression compared to the WT Cas12a protein, as demonstrated in Example 4. In some embodiments, the engineered Cas12a protein provided herein exhibits enhanced regulatory effect compared to the WT Cas12a protein, as demonstrated in Example 4.

In other embodiments, the engineered Cas12a protein provided herein can show improved epigenetic modifications compared to the WT Cas12a protein. In still other embodiments, the engineered Cas12a protein provided herein can have improved gene knockout, gene knock-in, and mutagenesis activities compared to the WT Cas12a protein. In further embodiments, the engineered Cas12a protein provided herein can show improved gene editing of single or multiple bases compared to the WT Cas12a protein. In yet other embodiments, the engineered Cas12a protein provided herein can have improved gene prime editing compared to the WT Cas12a protein.

In some embodiments, the engineered Cas12a protein provided herein is less susceptibility to variations in crRNA concentration compared to the WT Cas12a protein. In some embodiments, the engineered Cas12a protein provided herein exhibits increased level of activation under crRNA:Cas12a ratio of about 1:1 or lower compared to the WT Cas12a protein. For instance, see Examples 3 and 7. In some embodiments, the engineered Cas12a protein provided herein exhibits increased level of activation under crRNA:Cas12a ratio of about 1:0.9, about 1:0.8, about 1:0.7, about 1:0.6, about 1:0.5, about 1:0.4, about 1:0.3, about 1:0.2, about 1:0.1, or lower.

### Engineered Cas12a System

One aspect of the present disclosure relates to an engineered Cas12a system. The engineered Cas12a system has at least the following components: (a) one or more CRISPR RNAs (crRNAs) or a nucleic acid encoding each of the one or more crRNAs; and (b) the engineered Cas12a protein described herein or a nucleic acid encoding the Cas12a protein thereof.

As used herein, the term “CRISPR RNA” or “crRNA” refers to an RNA molecule having a synthetic sequence and typically comprising two sequence components: a spacer sequence and a guide RNA scaffold sequence (also called a “repeat sequence”). These two sequence components can be in a single RNA molecule or in a double-RNA molecule configuration (also known as a duplex guide RNA that comprises both a crRNA and a trans-activating crRNA (tracrRNA)). In some instances, the RNA molecule can have a crRNA component only (without a tracrRNA), for example, the RNAs that work with Cas12a. Thus, a crRNA as used herein generally comprises a repeat sequence and a spacer. In some instances, the repeat sequence is referred to as a “crRNA.”

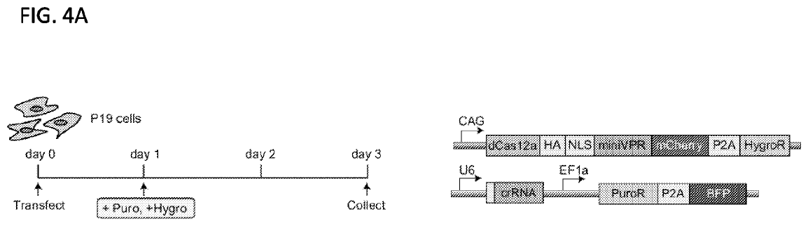

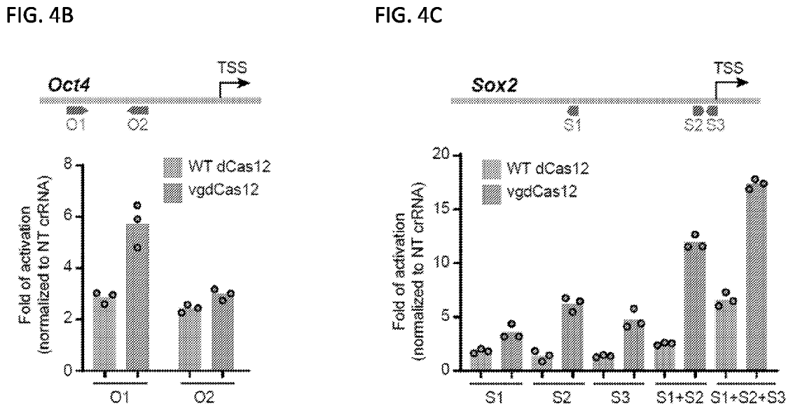

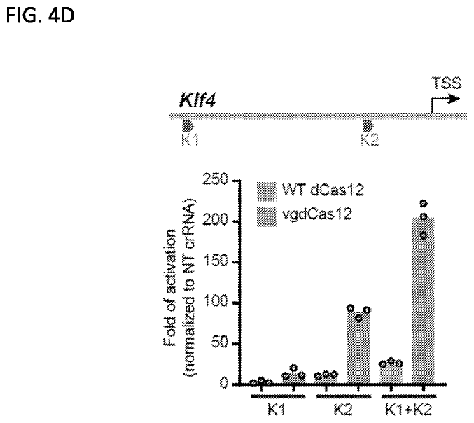

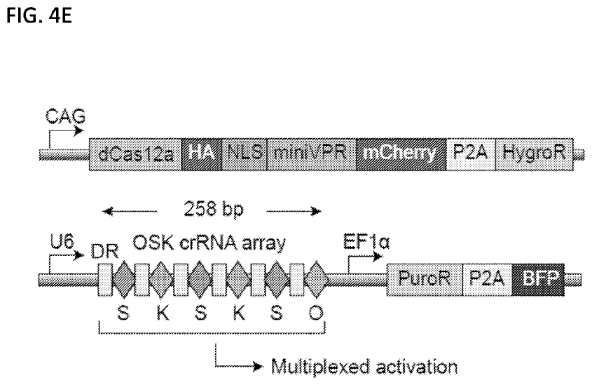

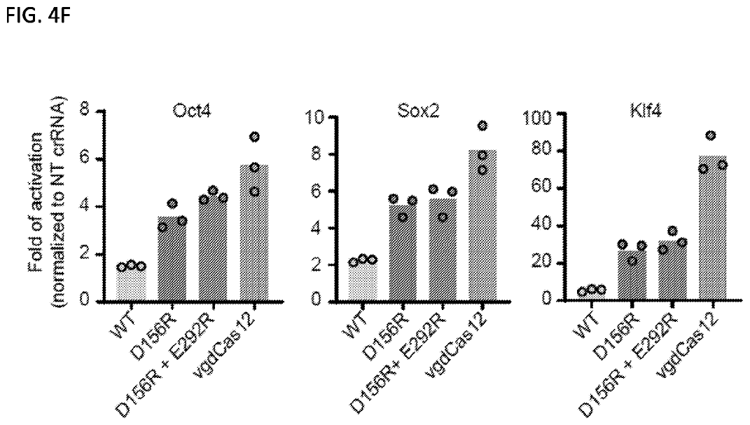

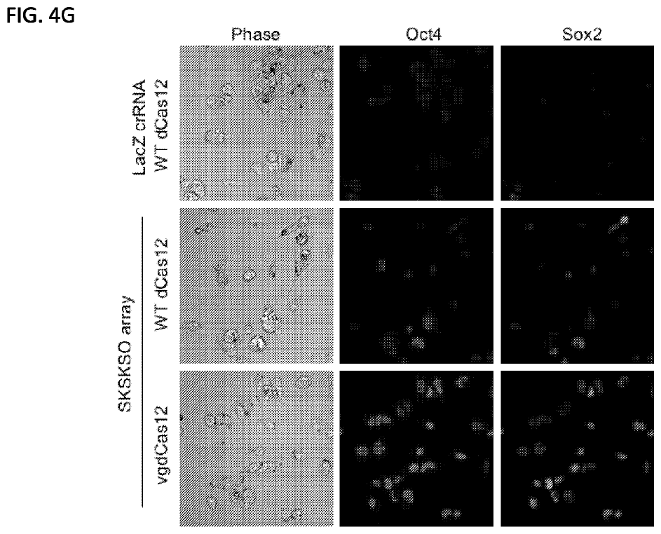

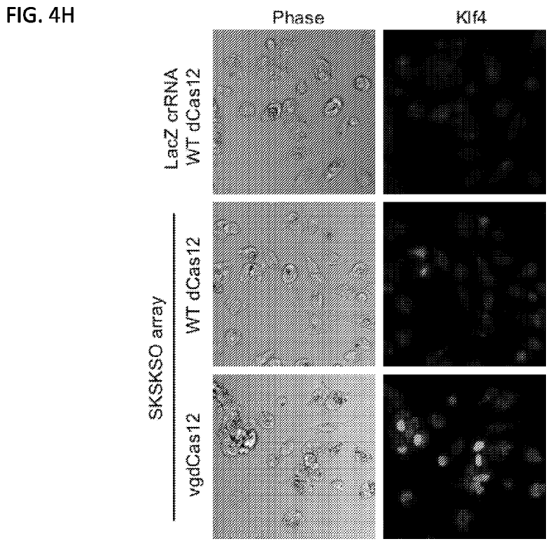

In some embodiments, the engineered Cas12a system can have more than one crRNAs, and each of the more than one crRNAs has a repeat sequence and a spacer. For instance, the engineered Cas12a system provided herein can have 2, 3, 4, 5, or more crRNAs. In some embodiments, the more than one crRNAs are arranged in tandem, i.e., located immediately adjacent to one another, and configures as a crRNA array. In some embodiments, the crRNA array can have 2-50 crRNAs. In other embodiments, the crRNA array can have 50-100 crRNAs. In some embodiments, the crRNA array can have 100-150 crRNAs. In other embodiments, the crRNA array can have 150-200 crRNAs. However, crRNAs containing more than 200 crRNAs are also contemplated by the present disclosure. An exemplary crRNA array and its application are illustrated in FIG. 4A and described in Example 8.

Each of the one or more crRNAs described herein comprises a repeat sequence and a spacer. The repeat sequence can be a Cas12a repeat sequence. In some embodiments, the repeat sequence is about 8-30 nucleotides long. In some embodiments, the repeat sequence is about 10-25 nucleotides long. In some embodiments, the repeat sequence is about 12-22 nucleotides long. In some embodiments, the repeat sequence is about 14-20 nucleotides long. In some embodiments, the repeat sequence is about 14-18 nucleotides long.

The spacer in a crRNA is configured to hybridize to a target nucleic acid. For instance, the spacer in a crRNA can have sequences that are complementary to its target nucleic acid sequence. The complementarity can be partial complementarity or complete (e.g., perfect) complementarity. The terms “complementary” and “complementarity” are used as they are in the art and refer to the natural binding of nucleic acid sequences by base pairing. The complementarity of two polynucleotide strands is achieved by distinct interactions between nucleobases: adenine (A), thymine (T) (uracil (U) in RNA), guanine (G), and cytosine (C). Adenine and guanine are purines, while thymine, cytosine, and uracil are pyrimidines. Both types of molecules complement each other and can only base pair with the opposing type of nucleobase by hydrogen bonding. For example, an adenine can only be efficiently paired with a thymine (A=T) or a uracil (A=U), and a guanine can only be efficiently paired with a cytosine (G≡C). The base complement A=T or A=U shares two hydrogen bonds, while the base pair G≡C shares three hydrogen bonds. The two complementary strands are oriented in opposite directions, and they are said to be antiparallel. For another example, the sequence 5′-A-G-T 3′ binds to the complementary sequence 3′-T-C-A-5′. The degree of complementarity between two strands may vary from complete (or perfect) complementarity to no complementarity. The degree of complementarity between polynucleotide strands has significant effects on the efficiency and strength of the hybridization between the nucleic acid strands. In some embodiments, the polynucleotide probes provided herein comprise two perfectly complementary strands of polynucleotides.

As used herein, the term “perfectly complementary” means that two strands of a double-stranded nucleic acid are complementary to one another at 100% of the bases, with no overhangs on either end of either strand. For example, two polynucleotides are perfectly complementary to one another when both strands are the same length, e.g., 100 bp in length, and each base in one strand is complementary to a corresponding base in the “opposite” strand, such that there are no overhangs on either the 5′ or 3′ end.

In some embodiments, the engineered Cas12a system comprises one or more crRNAs, and each spacer in at least a portion of the one or more crRNAs is configured to hybridize to the same target nucleic acid. In other embodiments, the engineered Cas12a system comprises one or more crRNAs, and each spacer in at least a portion of the one or more crRNAs is configured to hybridize to a different target nucleic acid. In certain embodiments, the engineered Cas12a system comprises one or more crRNAs, and each spacer in all of the one or more crRNAs is configured to hybridize to a different target nucleic acid.

The engineered Cas12a system provided herein is capable of binding to one or more target nucleic acids. As used herein, a “target nucleic acid sequence” of an engineered Cas12a system refers to a sequence to which a spacer sequence is designed to have complementarity, where hybridization between a target nucleic acid sequence and a spacer sequence promotes the formation of a CRISPR complex.

In some embodiments, the target nucleic acid refers to a nucleic acid of interest. For instance, the target nucleic acid can be a nucleic acid being investigated. In some embodiments, the target nucleic acid can be an endogenous gene. The target nucleic acids encompassed by the present disclosure can be RNAs and DNAs. In specific embodiments, the target nucleic acids can be DNAs, in particular, double-stranded DNAs (dsDNAs). Alternatively, the target nucleic acids can be derived from the genomic DNA, mitochondria DNA, chloroplast DNA, or viral DNA in host cells.

In some embodiments, the target nucleic acid refers to a genomic site or DNA locus capable of being recognized by and bound to a crRNA provided herein. An enzymatically active crRNA-Cas complex would process such a target site to result in a break at the CRISPR target site. In the case of a deactivated Cas, a crRNA-dCas still recognizes and binds a CRISPR target site without cutting the target nucleic acid (e.g., the target DNA).

In some embodiments, the target nucleic acid can be a transcription factor. In some embodiments, the target nucleic acid can be a metabolic enzyme. In other embodiments, the target nucleic acid can be any functional proteins. For example, in some embodiments, the target nucleic acid is involved in a pathological pathway, such as but not limited to, degenerative retinal diseases. Non-limiting examples of degenerative retinal diseases include Leber's congenital amaurosis, glaucoma, retinitis pigmentosa, and macular degeneration. In other embodiments, the target nucleic acid is involved in a biological pathway, such as but not limited to, aging, cell death, angiogenesis, DNA repair, and stem cell differentiation.

In some embodiments, the engineered Cas12a system provided herein can target any number of nucleic acids. In some embodiments, the engineered Cas12a system provided herein can target at least 2-4 different target nucleic acids. In some embodiments, the engineered Cas12a system provided herein can target at least 3 different target nucleic acids. In some embodiments, the engineered Cas12a system provided herein can target at least 5, at least 10, at least 15, at least 20, at least 25, at least 30 different target nucleic acids. In some embodiments, the engineered Cas12a system provided herein can target at least 50 different target nucleic acids. In other embodiments, the engineered Cas12a system provided herein can target at least 100 different target nucleic acids.

### Nucleic Acids and Vectors

Another aspect of the disclosure is one or more nucleic acids that encode the engineered Cas12a proteins and/or systems as described herein. As used herein, “encoding” refers to a polynucleotide encoding for the amino acids of a polypeptide, such as the engineered Cas12a proteins and/or systems described herein. A series of three nucleotide bases encodes one amino acid.

Some exemplary nucleic acid sequences are provided in Table 1. In one embodiment, the nucleic acid sequence provided herein encodes for the WT LbdCas12a as set forth in SEQ ID NO: 3. In some embodiments, the nucleic acid sequence is at least about 65%, 70%, 75%, 80%, 85%, 90%, 91%, 92%, 93%, 94%, 95%, 96%, 97%, 98%, 99%, or higher identity to a sequence set forth in SEQ ID NO: 3. In other exemplary embodiments, the nucleic acid sequence is at least about 80%, 90%, or 95% identical to a sequence set forth in SEQ ID NO: 3.

In another embodiment, the nucleic acid sequence provided herein encodes for the WT nuclease active form of lbCas12a as set forth in SEQ ID NO: 4. In some embodiments, the nucleic acid sequence is at least about 65%, 70%, 75%, 80%, 85%, 90%, 91%, 92%, 93%, 94%, 95%, 96%, 97%, 98%, 99%, or higher identity to a sequence set forth in SEQ ID NO: 4. In other exemplary embodiments, the nucleic acid sequence is at least about 80%, 90%, or 95% identical to a sequence set forth in SEQ ID NO: 4.

In yet another embodiment, the nucleic acid sequence provided herein encodes for the vgdCas12a protein as set forth in SEQ ID NO: 7. In some embodiments, the nucleic acid sequence is at least about 65%, 70%, 75%, 80%, 85%, 90%, 91%, 92%, 93%, 94%, 95%, 96%, 97%, 98%, 99%, or higher identity to a sequence set forth in SEQ ID NO: 7. In other exemplary embodiments, the nucleic acid sequence is at least about 80%, 90%, or 95% identical to a sequence set forth in SEQ ID NO: 7.

In still another embodiment, the nucleic acid sequence provided herein encodes for the nuclease active form of lbCas12a, vgCas12a protein, as set forth in SEQ ID NO: 8. In some embodiments, the nucleic acid sequence is at least about 65%, 70%, 75%, 80%, 85%, 90%, 91%, 92%, 93%, 94%, 95%, 96%, 97%, 98%, 99%, or higher identity to a sequence set forth in SEQ ID NO: 8. In other exemplary embodiments, the nucleic acid sequence is at least about 80%, 90%, or 95% identical to a sequence set forth in SEQ ID NO: 8.

As used herein, “expressed,” “expression,” or “expressing” refers to transcription of RNA from a DNA molecule. In some embodiments, the nucleic acid is operably linked to a heterologous nucleic acid sequence, such as, for example a structural gene that encodes a protein of interest or a regulatory sequence (e.g., a promoter sequence). As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s). In addition to promoters, regulatory elements include, without being limiting, an enhancer, a leader, a transcription start site (TSS), a linker, 5′ and 3′ untranslated regions (UTRs), an intron, a polyadenylation signal, and a termination region or sequence, etc., that are suitable, necessary or preferred for regulating or allowing expression of the gene or transcribable DNA sequence in a cell. Such additional regulatory element(s) can be optional and used to enhance or optimize expression of the gene or transcribable DNA sequence.

Also provided herein are vectors and/or plasmids containing one or more of the nucleic acids encoding the engineered Cas12a proteins and/or systems as described herein. As used herein, the terms “vector” or “plasmid” are used interchangeably and refer to a circular, double-stranded DNA molecule that is physically separate from chromosomal DNA. In one embodiment, a plasmid or vector used herein is capable of replication in vivo. In one embodiment, a plasmid provided herein is a bacterial plasmid. In one aspect, a plasmid or vector provided herein is a recombinant vector. As used herein, the term “recombinant vector” refers to a vector formed by laboratory methods of genetic recombination, such as molecular cloning. In another embodiment, a plasmid provided herein is a synthetic plasmid. As used herein, a “synthetic plasmid” is an artificially created plasmid that is capable of the same functions (e.g., replication) as a natural plasmid. Without being limited, one skilled in the art can create a synthetic plasmid de novo via synthesizing a plasmid by individual nucleotides, or by splicing together nucleic acids from different pre-existing plasmids. In other embodiments, the vector comprises a viral vector. In some embodiments, the viral vector comprises a lentiviral vector, an adeno virus vector, an adeno-associated viral vector, a piggyBac vector, herpes virus, simian virus 40 (SV40), bovine papilloma virus vectors, or a retroviral vector. Some embodiments disclosed herein relate expression cassettes including a nucleic acid molecule as disclosed herein.

In other embodiments, the present disclosure also provides expression cassettes containing one or more of the nucleic acids encoding the engineered Cas12a proteins as described herein. An expression cassettes is a construct of genetic material that contains coding sequences and enough regulatory information to direct proper transcription and/or translation of the coding sequences in a recipient cell, in vivo and/or ex vivo. The expression cassette may be inserted into a vector for targeting to a desired host cell. As such, the term “expression cassette” may be used interchangeably with the term “expression construct.”

A host cell as used herein can be a eukaryotic cell or prokaryotic cell. Non-limiting examples of eukaryotic cells include animal cell, plant cells, and fungal cells. In some embodiment, the eukaryotic cell comprises CHO, HEK293T, Sp2/0, MEL, COS, and insect cells. In some embodiment, the eukaryotic cell comprises mammalian cells. In some embodiment, the eukaryotic cell comprises human cells. In some embodiment, the prokaryotic cells comprises E. coli.

In some embodiments, the vector provided herein further comprises a promoter. As used herein, the term “promoter” generally refers to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A non-limiting exemplary inducible promoter includes a TRE promoter. A promoter can also be classified in terms of its origin, such as being heterologous, homologous, chimeric, synthetic, etc. A “heterologous” promoter is a promoter sequence having a different origin relative to its associated transcribable sequence, coding sequence, or gene (or transgene), and/or not naturally occurring in the plant species to be transformed. In some embodiments, the promoter can be a polymerase II promoter. Non-limiting, exemplary polymerase II promoter includes a CAG promoter, PGK promoter, CMV promoter, EF1α promoter, SV40 promoter, and Ubc promoter, ligand-inducible promoters (e.g., those can be conditionally activated by NFkB, NFAT, or externally supplied chemical compounds). In some embodiments, the CAG promoter is synthetic. In other embodiments, the promoter can be a polymerase III promoter. Non-limiting, exemplary polymerase III promoter includes the mouse U6 promoter, the human U6 promoter, the H1 promoter, and the 7SK promoter.

In some embodiments, the vector provided herein further comprises a reporter gene. For example, the reporter gene can be, without limitations, BFP, GFP, and mCherry. A skilled person knows how to choose or design reporter genes.

The nucleic acids described herein can be contained within a vector that is capable of directing their expression in, for example, a cell that has been transduced with the vector. Suitable vectors for use in eukaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd Ed. (1989).

The vectors are useful for autonomous replication in a host cell or may be integrated into the genome of a host cell upon introduction into the host cell, and thereby are replicated along with the host genome (e.g., non-episomal mammalian vectors).

In some embodiments, the vector is an expression vector. Expression vectors are capable of directing the expression of coding sequences to which they are operably linked. In some embodiments, the vector is eukaryotic expression vector, i.e. the vector is capable of directing the expression of coding sequences to which they are operably linked in a eukaryotic cell. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are also included.

DNA vectors can be introduced into eukaryotic cells via conventional transformation or transfection techniques. Suitable methods for transforming or transfecting host cells can be found in Sambrook et al. (1989) Molecular Cloning: A Laboratory Manual (2nd ed., Cold Spring Harbor Laboratory Press, Plainview, N.Y.) and other standard molecular biology laboratory manuals.

In some embodiments, the vector is a viral vector. The term “viral vector” is widely used to refer either to a nucleic acid molecule that includes virus-derived nucleic acid elements that typically facilitate transfer of the nucleic acid molecule or integration into the genome of a cell, or to a viral particle that mediates nucleic acid transfer. Viral particles typically include viral components, and sometimes also host cell components, in addition to nucleic acid(s). Retroviral vectors used herein contain structural and functional genetic elements, or portions thereof, that are primarily derived from a retrovirus. Retroviral lentivirus vectors contain structural and functional genetic elements, or portions thereof including LTRs, that are primarily derived from a lentivirus (a sub-type of retrovirus).

In some embodiments, the nucleic acids are delivered by non-viral delivery vehicles known in the art. For example, the nucleic acid molecule can be stably integrated in the host genome, or can be episomally replicating, or present in the recombinant host cell as a mini-circle expression vector for stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can also be accomplished using classical random genomic recombination techniques or with more precise genome editing techniques such as using guide RNA-directed CRISPR/Cas9, DNA-guided endonuclease genome editing NgAgo (Natronobacterium gregoryi Argonaute), or TALENs genome editing (transcription activator-like effector nucleases). In some embodiments, the nucleic acid molecule is present in the recombinant host cell as a mini-circle expression vector for stable or transient expression.

The nucleic acids can be encapsulated in a viral capsid or a lipid nanoparticle. For example, introduction of nucleic acids into cells may be achieved using viral transduction methods. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also useful for nucleic acid delivery and gene therapy via viral transduction. Lentiviral vectors offer several attractive properties as gene-delivery vehicles, including: (i) sustained gene delivery through stable vector integration into the host cell genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile (e.g., by targeting a site for integration that has little or no oncogenic potential); and (vii) a relatively easy system for vector manipulation and production.

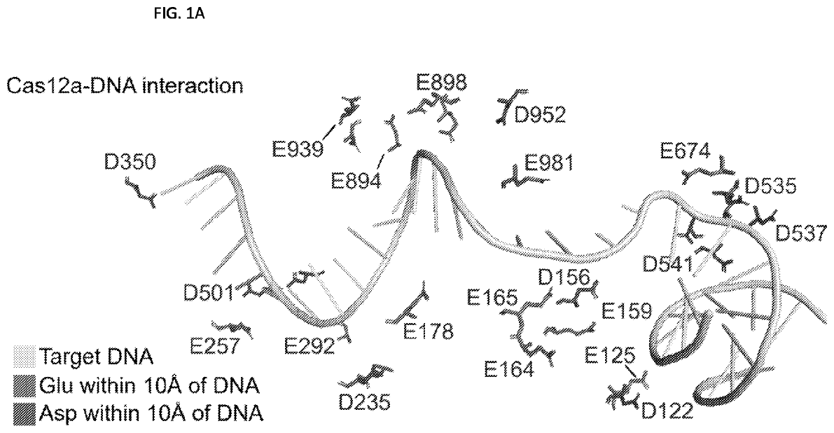

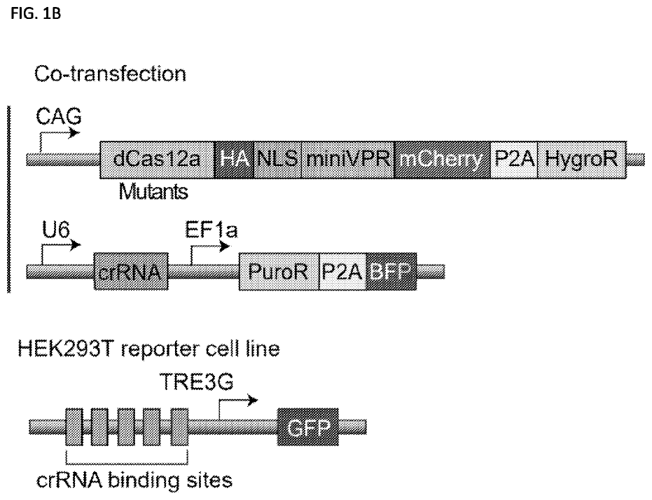

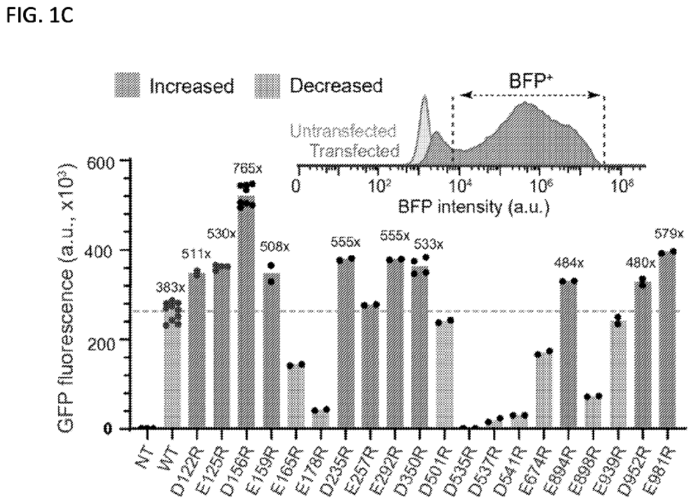

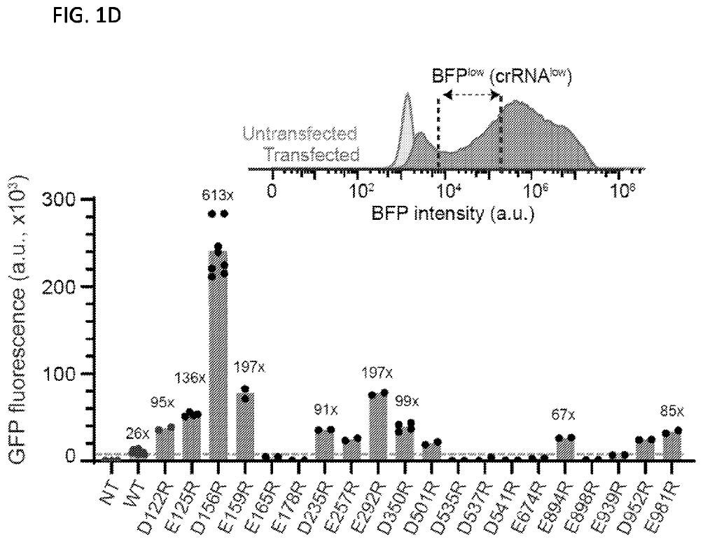

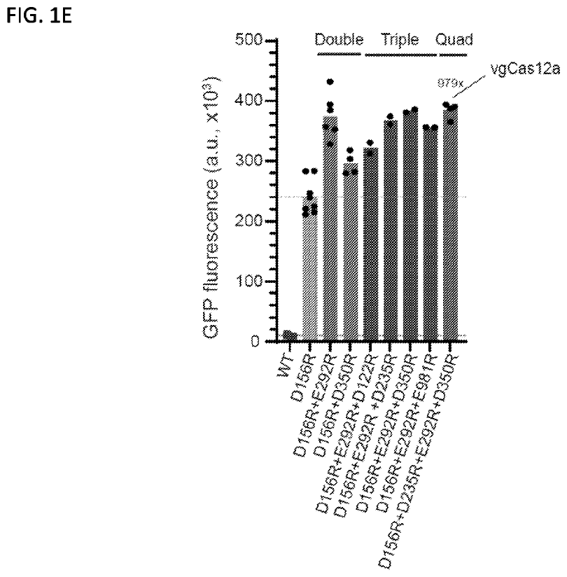

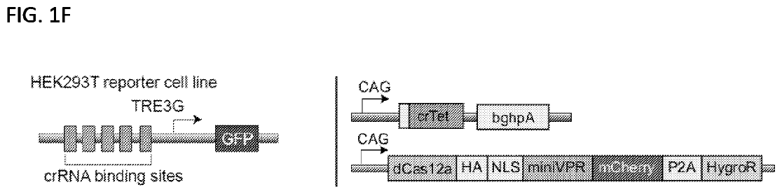

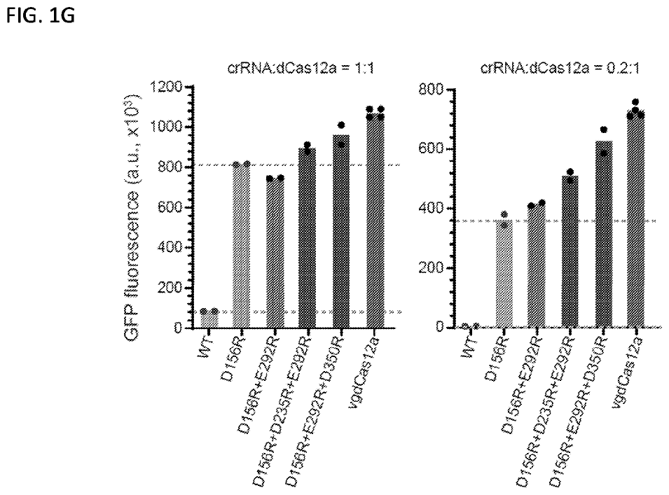

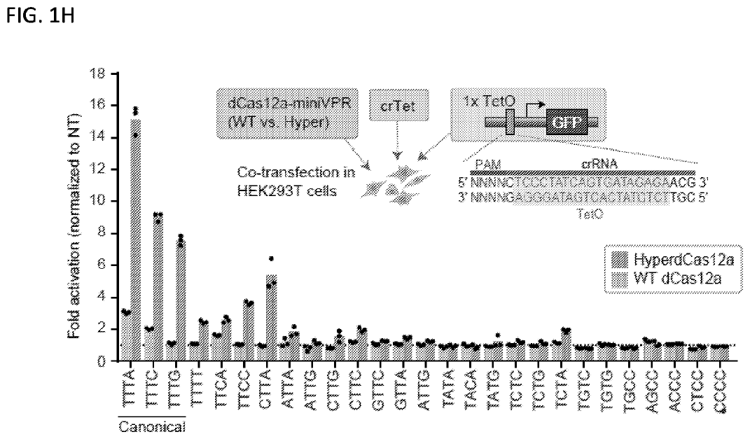

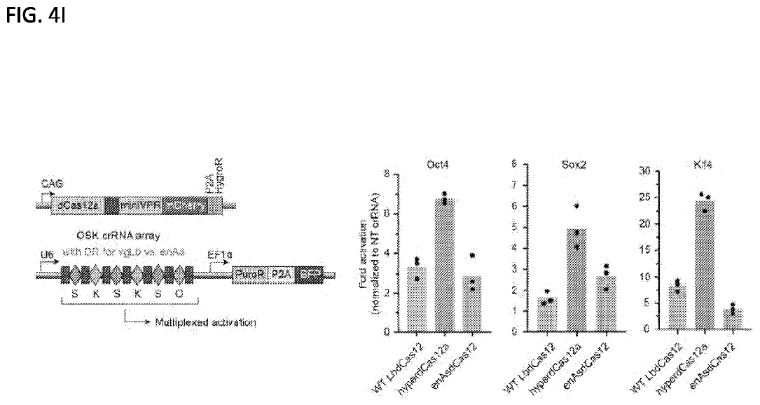

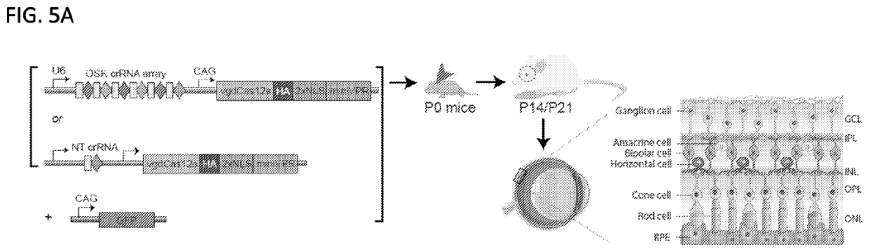

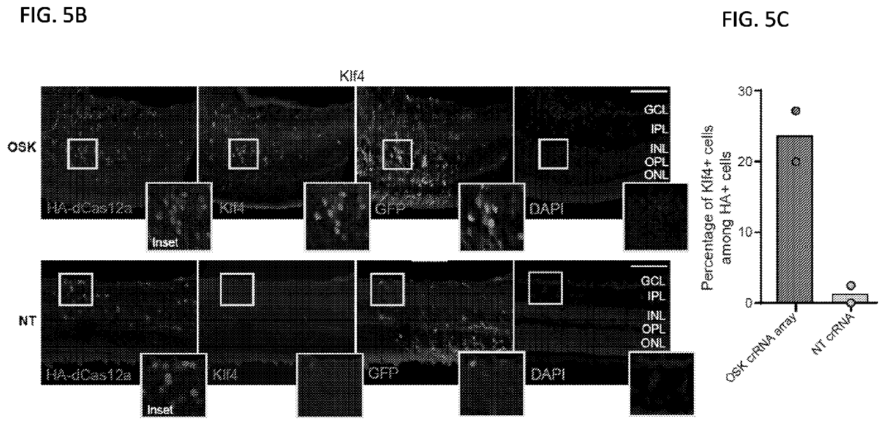

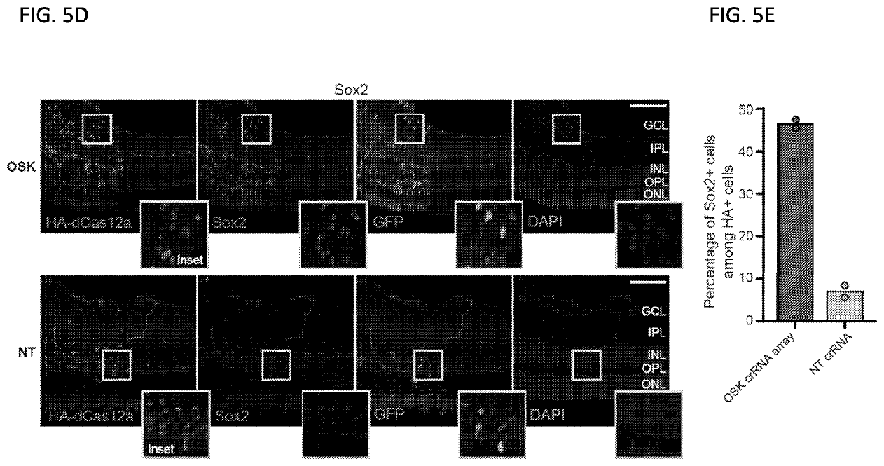

One aspect of the present disclosure provides an engineered Cas12a system in the form of one or more expression vectors. In some embodiments, the one or more crRNAs and the engineered Cas12a protein of the engineered Cas12a system can be located in separate vectors. For instance, an example of an engineered Cas12a system of which the one or more crRNAs and the engineered Cas12a protein are located in different vectors is illustrated in FIGS. 1B, 1F, 2A, 2C, 2E, 4A, 3E, and 11A. While in other embodiments, the one or more crRNAs and the engineered Cas12a protein of the engineered Cas12a system can be located in the same vector. For instance, an example of an engineered Cas12a system of which the array of crRNAs and the engineered Cas12a protein are located in the same vector is illustrated in FIG. 5A.

The expression of the one or more crRNAs or the Cas12a protein can be driven by an RNA polymerase III promoter, an RNA polymerase II promoter, an inducible promoter, or a combination thereof, as described herein.

In some specific embodiments, the one or more crRNAs and the Cas12a protein can be located in the same vector, and the expression of the one or more crRNAs or the Cas12a protein is driven by the same promoter, for example, see FIG. 5A. In other embodiments, the one or more crRNAs and the Cas12a protein can be located in the same vector, and the expression of the one or more crRNAs or the Cas12a protein is driven by different promoters.

In other specific embodiments, the one or more crRNAs and the Cas12a protein can be located in different vectors, and the expression of the one or more crRNAs or the Cas12a protein is driven by different promoters, for example, see FIGS. 1B, 2A, 2C, 2E, 4A, 3E, and 11A.

In other specific embodiments, the one or more crRNAs and the Cas12a protein can be located in different vectors, and the expression of the one or more crRNAs or the Cas12a protein is driven by the same promoter, for example, see FIG. 1F.

### Pharmaceutical Composition

The present disclosure further provides pharmaceutical compositions comprising the engineered Cas12a proteins, the nucleic acids, the vectors, or the engineered Cas12a systems described herein. in some embodiments, the pharmaceutical compositions further comprise one or more pharmaceutically acceptable excipient or carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable excipient include physiological saline, bacteriostatic water, Cremophor EL™. (BASF, Parsippany, N.J.), or phosphate buffered saline (PBS). In all cases, the composition should be sterile and should be fluid to the extent that it can be administered by syringe. It should be stable under the conditions of manufacture and storage and must be preserved against the contaminating action of microorganisms such as bacteria and fungi. The excipient can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the engineered Cas12a proteins, the nucleic acids, the vectors, or the engineered Cas12a systems of the disclosure can be administered by transfection or infection with nucleic acids encoding them, using methods known in the art, including but not limited to the methods described in McCaffrey et al., Nature (2002) 418:6893, Xia et al., Nature Biotechnol (2002) 20:1006-10, and Putnam, Am J Health Syst Pharm (1996) 53:151-60, erratum at Am J Health Syst Pharm (1996) 53:325.

### Engineered Cells

Another aspect of the present disclosure encompasses engineered cells or recombinant cells. In some embodiments, the engineered Cas12a proteins, the nucleic acids, the vectors, or the engineered Cas12a systems of the disclosure can be used in eukaryotic cells, such as mammalian cells, for example, human cells, to produce engineered cells with modulated expression of target nucleic acids. Any human cell is contemplated for use with the engineered Cas12a proteins, the nucleic acids, the vectors, or the engineered Cas12a systems of the disclosure disclosed herein.

In some embodiments, the cells are engineered to express the engineered Cas12a proteins, the nucleic acids, the vectors, or the engineered Cas12a systems described herein. In some embodiments, an engineered cell ex vivo or in vitro includes: (a) nucleic acid encoding the one or more CRISPR RNAs described herein, and/or (b) nucleic acid encoding the engineered Cas12a protein described herein.

Some embodiments disclosed herein relate to a method of engineering a cell that includes introducing into the cell, such as an animal cell, the engineered Cas12a proteins, the nucleic acids, the vectors, or the engineered Cas12a systems as described herein, and selecting or screening for an engineered cell transformed by the engineered Cas12a proteins, the nucleic acids, the vectors, or the engineered Cas12a systems. The term “engineered cell” or “recombinant cells” refers not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. Techniques for transforming a wide variety of cell are known in the art.

In a related aspect, some embodiments relate to engineered cells or recombinant cells, for example, engineered animal cells that include a heterologous nucleic acid and/or polypeptide as described herein. The nucleic acid can be stably integrated in the host genome, or can be episomally replicating, or present in the engineered cell as a mini-circle expression vector for stable or transient expression.

In some embodiments, provided herein is an engineered cell, e.g., an isolated engineered cell, prepared by modulating the expression of a target gene in a target nucleic acid or otherwise modifying the target nucleic acid in a cell according to any of the methods described herein, thereby producing the engineered cell. In some embodiments, provided herein is an engineered cell prepared by a method comprising providing to a cell the engineered Cas12a proteins, the nucleic acids, the vectors, or the engineered Cas12a systems as described herein.

In some embodiments, according to any of the engineered cells described herein, the engineered cell is capable of expressing or not expressing target nucleic acids (e.g., target DNAs). In some embodiments, according to any of the engineered cells described herein, the engineered cell is capable of regulated expression of target nucleic acids. In some embodiments, according to any of the engineered cells described herein, the engineered cell exhibits altered expression pattern of target nucleic acids. In other embodiments, the engineered cells described herein exhibits desired phenotypes because of the altered expression pattern of target nucleic acids.

### Kits

In some embodiments, provided herein are kits for carrying out a method described herein. A kit can include one or more components of the engineered Cas12a proteins, the nucleic acids, the vectors, or the engineered Cas12a systems as described herein. A kit as described herein can further include one or more additional reagents, where such additional reagents can be selected from: a buffer for introducing one or more components of the engineered Cas12a proteins, the nucleic acids, the vectors, or the engineered Cas12a systems into a cell; a dilution buffer; a reconstitution solution; a wash buffer; a control reagent; a control expression vector or polyribonucleotide; a reagent for in vitro production of one or more components of the engineered Cas12a proteins, the nucleic acids, the vectors, or the engineered Cas12a systems, and the like.

Components of a kit can be in separate containers; or can be combined in a single container.

In addition to the above-mentioned components, a kit can further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (e.g., associated with the packaging or sub-packaging) etc. In some embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### III. Methods

### Methods of Targeting Nucleic Acids

Provided herein are methods of targeting (e.g., binding to, modifying, detecting, etc.) one or more target nucleic acids (e.g., dsDNA or RNA) using the engineered Cas12a proteins, the nucleic acids, the vectors, or the engineered Cas12a systems provided herein.

In some embodiments, provided herein is a method of targeting (e.g., binding to, modifying, detecting, etc.) a target nucleic acid in a sample comprising introducing into the sample the components of the engineered Cas12a proteins, the nucleic acids, the vectors, or the engineered Cas12a systems as described herein.

Targeting a nucleic acid molecule can include one or more of cutting or nicking the target nucleic acid molecule; modulating the expression of a gene present in the target nucleic acid molecule (such as by regulating transcription of the gene from a target DNA or RNA, e.g., to downregulate and/or upregulate expression of a gene); visualizing, labeling, or detecting the target nucleic acid molecule; binding the target nucleic acid molecule, editing the target nucleic acid molecule, trafficking the target nucleic acid molecule, and masking the target nucleic acid molecule. In some embodiments, modifying the target nucleic acid molecule includes introducing one or more of a nucleobase substitution, a nucleobase deletion, a nucleobase insertion, a break in the target nucleic acid molecule, methylation of the target nucleic acid molecule, and demethylation of the nucleic acid molecule. In some embodiments, such methods are used to treat a disease, such as a disease in a human. In such embodiments, one or more target nucleic acids are associated with the disease.

### Methods of Gene Modulation

The engineered Cas12a proteins, the nucleic acids, the vectors, or the engineered Cas12a systems provided herein can be used to modulate (e.g., activate, repress, silence, knockdown, or knockout) gene expression in a sample. The modulation can be done in vitro or in vivo. The gene expression to be modulated can be endogenous or exogenous gene expression.

In some embodiments, the present disclosure describes a method for improving multi-gene expression control in in the sample. In some embodiments, the present disclosure provides a method for simultaneous activation or repression of multiple target nucleic acids (e.g., endogenous genes). In some embodiments, the modulating results in transcriptional activation of the one or more target nucleic acids. In other embodiments, the modulating results in transcriptional repression of the one or more target nucleic acids.

In some embodiments, the present disclosure describes methods of modulating one or more target nucleic acids (e.g., endogenous genes) in a sample. In some embodiments, the methods of modulating one or more target nucleic acids (e.g., endogenous genes) in a sample as provided herein involves contacting the sample (such as the one or more cells) with the engineered Cas12a proteins, the nucleic acids, the vectors, or the engineered Cas12a systems provided herein. The contacting can occur in vitro, in vivo, or ex vivo. In some embodiments, the methods comprise modulating the more than one target nucleic acids simultaneously. In certain embodiments, the modulating can result in transcriptional activation of the one or more target nucleic acids. See, for instance, Examples 1, 3, 6, and 7. In other embodiments, the modulating can result in transcriptional repression of the one or more target nucleic acids. See, for instance, Example 4. In some exemplary embodiments, the modulating can result in epigenetic modifications. Non-limiting exemplary epigenetic modifications encompassed by the present disclosure include targeted CpG methylation, histone H2, H3 or H4 methylation, or acetylation of the one or more target nucleic acids. In some exemplary embodiments, the modulating can be applied for gene editing. For instance, the modulating can result in editing single or multiple bases of the one or more target nucleic acids. Alternatively, the modulating can result in altered expression of the one or more target nucleic acids. Furthermore, the modulating the target nucleic acid in the sample results in depletion of the one or more target nucleic acids. See, for instance, Example 4. In addition, the modulating can result in reprograming the lineage of the sample. An illustrative application is shown in Example 8 of the present disclosure, which demonstrates that the in vivo multiplex activation by vgdCas12a in mouse retina leads to progenitor cell differentiation.

As one skilled in the art would appreciate, the one or more target nucleic acids that can be modulated by the present disclosure can include any nucleic acids encoding functional proteins. A “functional protein” as used herein generally refers to proteins that have biological activity. For instance, a functional protein can be a structural protein. In other embodiments, a functional protein can be involved in disease and physiology, drug interaction, aging, cell differentiation, etc. Alternatively, a functional protein can be involved in any of the biological pathways, including without being limited to, the metabolic pathway, any genetic pathways, or a signal transduction pathway. Multiple pathway databases are freely accessible in the field. For example, PathBank provides a list of various pathway databases, which is accessible at https://pathbank.org/others. In some exemplary embodiments, the one or more target nucleic acids that can be modulated by the present disclosure comprise one or more nucleic acids encoding transcriptional factors and/or metabolic enzymes.

### Methods of Treatment

Another aspect of the disclosure relates to methods of treatment. Specifically, the pharmaceutical compositions provided herein can be used to treat various disorders (or diseases, symptoms, or pathological conditions). In one embodiment, the present disclosure provides a method for treating a disorder in an individual in need thereof. In other embodiments, the methods of treating involves administering a therapeutically effective dose of the pharmaceutical composition provided herein.

The disorder to be treated by the methods provided herein can be a genetic disorder. The term “genetic disorder” is used as its common meaning in the field, and generally refers to a health problem caused by one or more abnormalities in the genome of an individual. An genetic disorder can be caused by a mutation in a single gene (monogenic) or multiple genes (polygenic) or by a chromosomal abnormality. In some embodiments, the disorder is monogenic. In other embodiments, the disorder is polygenic.

Some non-limiting exemplary disorders that can be treated by the methods provided herein include inherited retinal degenerative disorders, inherited optic nerve disorders, and polygenic degenerative diseases of the eye. Exemplary inherited retinal degenerative disorders include, but are not limited to, Leber's congenital amaurosis and retinitis pigmentosa. Exemplary inherited optic nerve disorders include, but are not limited to, Leber's hereditary optic neuropathy and autosomal dominant optic neuropathy. Exemplary polygenic degenerative diseases include, but are not limited to, glaucoma and macular degeneration.

The methods of treating of the present disclosure can be in the form of a gene therapy. In some embodiments, the methods of treating involves modifying one or more target nucleic acids in a cell by introducing into the cell a pharmaceutical composition comprising the engineered Cas12a protein, the nucleic acid, the vector, or the engineered Cas12a system as described herein.

The discussion of the general methods given herein is intended for illustrative purposes only. Other alternative methods and alternatives will be apparent to those of skill in the art upon review of this disclosure, and are to be included within the spirit and purview of this application.

### EXAMPLES

### Example 1: Synthetic Cas12a for Enhanced Multiplex Gene Control and Editing

The purpose of this example is to describe experiments showing that variants of LbdCas12a exhibit increased activity over the wildtype protein. If mutants were screened randomly, it would be expected that most mutations would decrease or abolish protein function. Instead, by using a protein-structure-guided design and focusing on negatively charged amino acid residues on Cas12a within close proximity to target DNA, then systematically mutating each sidechain to a positively charged one (FIG. 1A), it may be possible to increase affinity of the Cas protein to its target DNA. While most mutations tested worsened or decreased protein activity, a few mutants (specifically, D122R, E125R, D156R, E159R, D235R, E257R, E292R, D350R, E894R, D952R, and E981R) enhanced dCas12a activity (FIGS. 1B-1C). Also investigated were the effects of these mutants at lower Blue Fluorescent Protein (BFP) intensity (FIG. 1D), which serves as a proxy for conditions with low reactant concentrations (i.e., concentrations of crRNA and Cas12a protein), which may be particularly relevant for in vivo delivery.

Notably, it was observed that the enhancement of dCas12a activity of several of these mutants was especially evident at these low reactant concentrations. Several of the mutants achieved a 3-23×-fold increase in activation above the wildtype (WT) protein (FIG. 1D). Furthermore, combining 4 of the best-performing mutants (D156R, D235R, E292R, and D350R) were shown to achieve further increase in activation with several permutations of combinatorial mutants (FIG. 1E). In addition to crRNAs driven by type III RNA polymerase III promoter, such as U6 (FIGS. 1A-1E), also tested was the functionality of these Cas12a mutants with crRNA driven by an RNA polymerase II promoter, such as the synthetic CAG promoter. Using dCas12a for multiplex genome regulation applications would require that the protein maintains its RNAse ability to process a functional crRNA from a longer poly-crRNA transcript. To easily test this using the same GFP reporter system, we compared the performance of the dCas12a mutants to the WT protein using crRNA expressed by an RNA polymerase II promoter (CAG promoter, in this case), so that dCas12a would be required to process the crRNA before activation of the target gene. It was shown that the mutants exhibited improved activation compared to WT in this context as well. Notably, a combinatorial mutant consisting of 4 of the best-performing mutants (from FIG. 1E) achieved the highest level of activation, and this was particularly striking under conditions of low crRNA:Cas12a ratio, which would be most relevant for in vivo conditions (FIGS. 1F-G). It is worth noting here that while the WT protein (and to a lesser extent, the single D156R mutant) showed a decrease in activation when crRNA amount was decreased 5-fold, the mutant incorporating quadruple mutations showed much less decrease, indicating that it is less susceptible to variations in crRNA concentration. This quadruple mutant is heretofore referred to as vgdCas12a (very good dCas12a).

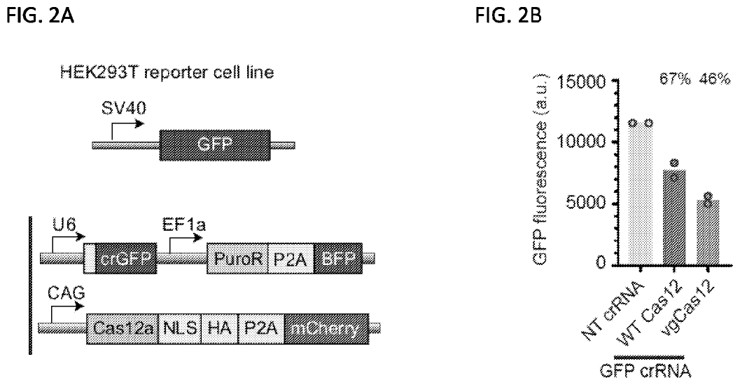

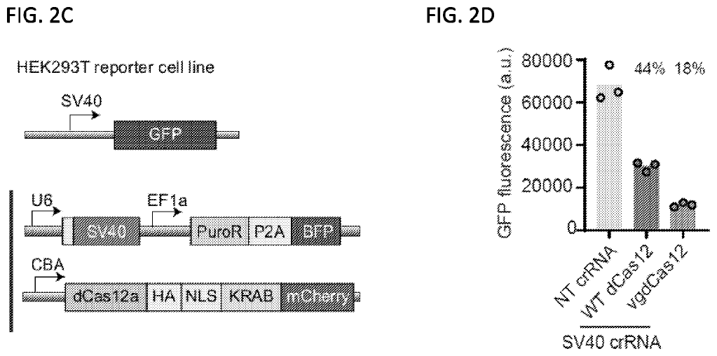

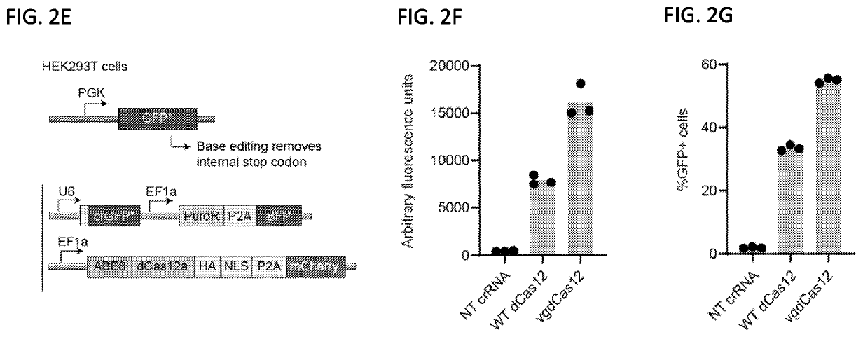

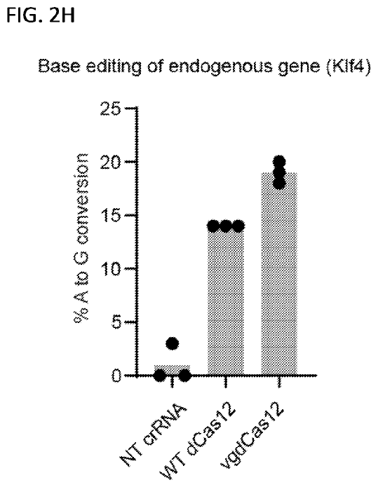

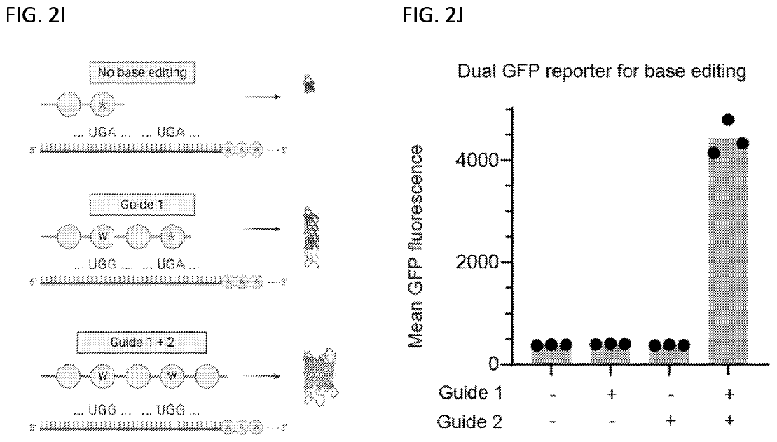

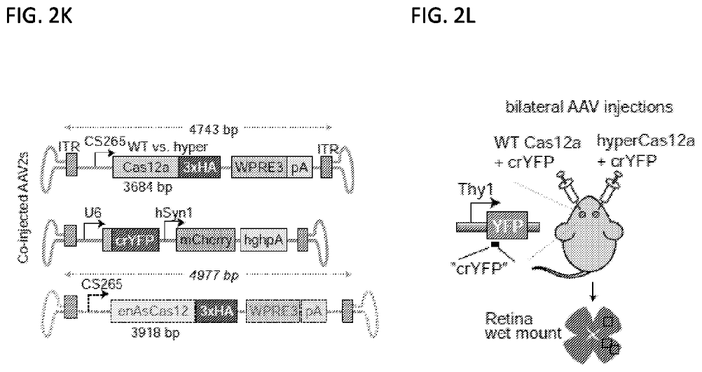

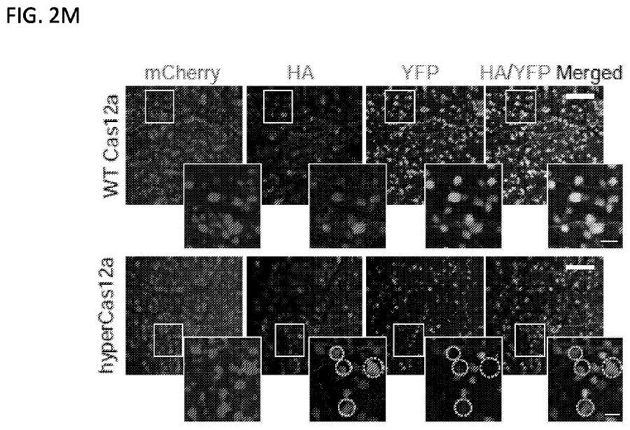

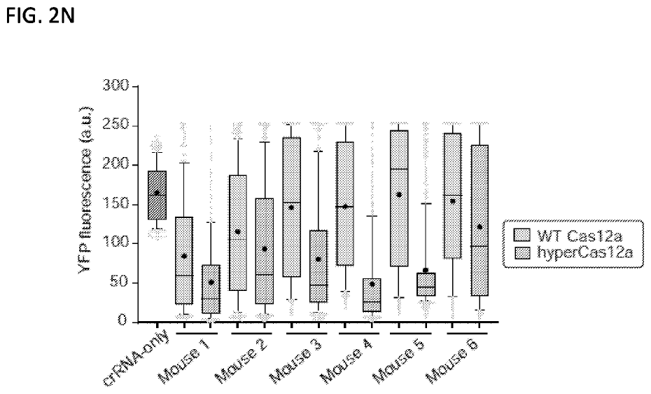

It was further shown that vgCas12a also works for better gene editing. The four activity enhancing mutations described previously were introduced into the nuclease-active form of Cas12a, and it was shown that vgCas12a enables more effective GFP knockout in SV40-GFP reporter cells (FIGS. 2A-2B). Furthermore, vgdCas12a can be modularly coupled to different effectors and exhibit enhanced regulatory effects. For example, when coupled to a transcriptional repressor, the mutant fusion protein enabled ˜82% repression over non-targeting control, compared to only 56% by its wildtype equivalent (FIGS. 2C-2D).

It was further investigated whether the variant protein allows better multiplexed gene regulation. Co-expression of a single CRISPR-RNA (crRNA) array encoding 6 crRNAs activated three endogenous genes, Oct4, Sox2, and Klf4, and it was shown that vgdCas12a-miniVPR exhibited a dramatically higher magnitude of transcriptional activation as compared to the wildtype equivalent (FIG. 4F). Additionally, the enhanced performance of vgdCas12a over the single D156R mutant and the double D156R/E292R mutant in this assay highlights the synergistic power of our combinatorial mutations, and points to vgdCas12a as a logical protein of choice for multiplex genome engineering in mammalian cells.

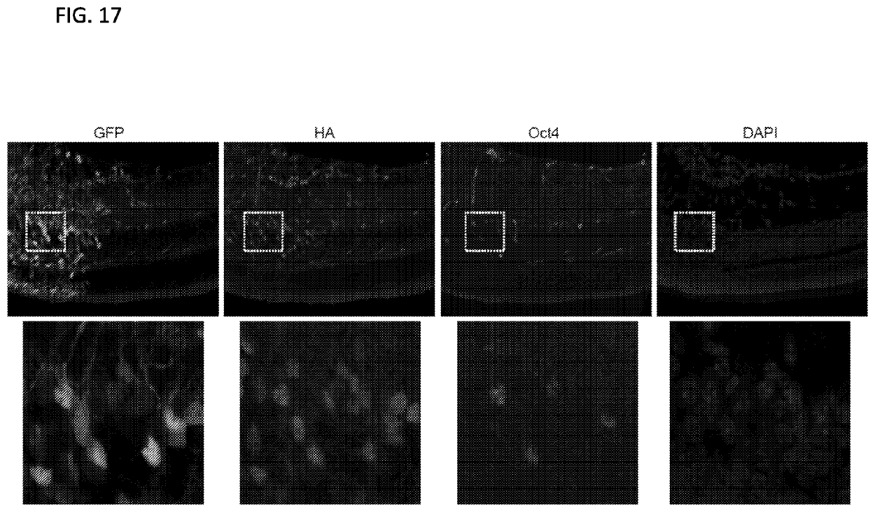

The retina was targeted for in vivo delivery, given the high interest in using genome engineering for ocular disorders, due to its relative immune privilege and accessibility, as well as the global burden of degenerative retinal diseases. Using the well-validated in vivo electroporation technique (FIGS. 5A-5B), expression of HA-tagged vgdCas12a-miniVPR was robustly detected at 14 days after delivery in multiple layers of the retina (FIGS. 5C-5D). Described and illustrated herein is evidence that vgdCas12a-miniVPR, when co-delivered with a crRNA array, can simultaneously activate target genes Klf4 and Sox2 in the postnatal murine retina (FIG. 5B-5E), and Oct4 to a lesser extent (FIG. 17).

### Example 2: Methods

This Example described the methods used in the present disclosure.

### Cell Culture:

HEK293T cells (Clontech Laboratories, Mountain View, CA) were cultured in DMEM+GlutaMAX (Thermo Fisher Scientific, Waltham, MA) supplemented with 10% FBS (ALSTEM, Richmond, CA) and 100 U/mL of penicillin and streptomycin (Life Technologies, Carlsbad, CA). P19 cells were cultured in alpha-MEM with nucleosides (Invitrogen, Carlsbad, CA) with same FBS and pen/strep as above. Cells were maintained at 37° C. and 5% CO₂ and passaged using standard cell culture techniques. For transient transfection of HEK293T cells, cells were seeded the day before transfection at 1×10⁵ cells/mL. Transient transfections were performed using 3 mL of TransIT-LT1 transfection reagent (Mirus Bio, Madison, WI) per mg of plasmid. Cells were analyzed 2 days post transfection, as indicated. For transient transfection of P19 cells, cells were seeded the day before transfection at density of 2×10⁵ cells/mL. Transient transfections were performed using 3 ul of Mirus X2 transfection reagent (Mirus Bio, Madison, WI) per μg of plasmid. For double-selection, cells were treated with 500 μg/ml of hygromycin and 2 μg/ml of puromycin. Cells were analyzed 3 days post transfection, as indicated.

### Plasmid Cloning

Standard molecular cloning techniques were used to assemble constructs in this disclosure. Nuclease-dead dCas12a from Lachnospiraceae bacterium and its crRNA backbone were modified from methods described in Kempton, H. R. et al. Short Article Multiple Input Sensing and Signal Integration Using a Split Cas12a System Short Article Multiple Input Sensing and Signal Integration Using a Split Cas12a System. Mol. Cell 1-8 (2020) doi:10.1016/j.molcel.2020.01.016.

### Flow Cytometry

Cells were dissociated using 0.05% Trypsin-EDTA (Life Technologies, Carlsbad, CA), resuspended in PBS+10% FBS, and analyzed for fluorescence using a CytoFLEX S flow cytometer (Beckman Coulter, Brea, CA). 10,000 cells from the population of interest (for most experiments, mCherry+ and BFP+ gated based on non-transfected control) were collected for each sample and analyzed using FlowJo.

qPCR (Quantification of mRNA Expression)

RNA was isolated from transfected cells using Qiagen RNeasy plus kit (Qiagen, Hilden, Germany) followed by reverse transcription of 100 ng RNA into cDNA using iScripst kit (Bio-Rad Laboratories, Hercules, CA). A Quantitative PCR (qPCR) reaction was performed using SYBR master mix (Bio-Rad Laboratories, Hercules, CA) according to the manufacturer's protocol. Quantification of RNA expression was normalized based on expression of glyceraldehyde 3-phosphate dehydrogenase and calculated using ΔΔCt.

### Immunostaining

P19 cells were seeded onto black flat-bottom 96-well plates at 48 hr after transfection (continuing in dual selection media), fixed with 1×DPBS/4% formaldehyde 24 hr after seeding. Each well was permeabilized with 1×DPBS/0.25% Triton X-100 and blocked with 1×DPBS/5% donkey serum, then incubated at 4 C overnight with primary antibodies diluted in 1×DPBS/5% donkey serum: mouse anti-Oct4 (1:200, BD bioscience, 611203), rabbit anti-Sox2 (1:200, Cell signaling, 14962), and goat anti-Klf4 (1:200, R&D system, AF3158). Each well was washed 3× with 1×DPBS then incubated for 1 hr with Alexa Fluor-conjugated 488 or 647 donkey secondary antibodies (Life Tech) at 1:500 diluted in same buffer as primary antibodies. Each well was then washed 3× with 1×PBS, and each well is immersed in 1×PBS in each well. No nuclear dye was used. Imaging was done with Leica DMi8 inverted microscope with 20× objective and a Leica DFC9000 CT camera.

### RNAseq

HEK reporter cell line stably expressing TRE3G-GFP were seeded in a 6 well plate at density of 2×10⁵/ml and were co-transfected next day with TET crRNA or LacZ non-target crRNA with dCas12aWT or vgdCas12a, in duplicates. One day after transfection, transfected cells were placed in antibiotic selection (hygromycin 500 μg/ml and puromycin 2 μg/ml) for 2 days before harvest. Total RNA was isolated by using RNeasy Plus Mini Kit (QIAGEN). Library preparation and next-generation sequencing were performed by Novogene (Chula Vista, CA) as described previously. Spliced Transcripts Alignment to a Reference (STAR) software was used to index hg19 genome and GFP sequence, and then to map paired end reads to the genome. HTSeq-Count was used to quantify gene-level expression. Gene-level fragments per kilobase of transcript per million mapped reads (FPKM) were calculated using a custom Python script. The script is available at https://github.com/QilabGitHub/FPKMcalculation.

### Animals

Wild-type neonatal mice were obtained from timed pregnant CD1 mice (Charles River Laboratories, Wilmington, MA). For AAV experiments, Thy1-YFP-17 transgenic mice were originally generated by Drs. Guoping Feng and Josh Sanes (Feng, G. et al. Imaging Neuronal Subsets in Transgenic Mice Expressing Multiple Spectral Variants of GFP. Neuron 28, 41-51 (2000)) and were acquired from Dr. Zhigang He; male mice age 6-8 weeks were used. All animal studies were approved by the Institutional Animal Care and Use Committee at Stanford School of Medicine.

### In Vivo Plasmid Electroporation

In vivo retina electroporation was carried out as described in Wang, S., Sengel, C., Emerson, M. M. & Cepko, C. L. A gene regulatory network controls the binary fate decision of rod and bipolar cells in the vertebrate retina, Dev. Cell 30, 513-527 (2014). Plasmid with wildtype dCas12a was mixed with CAG-GFP construct in ˜5:1 ratio and electroporated at a concentration of up to 2 μg/μl total plasmid at P0. Five pulses of 80 V, 50 ms each at intervals of 950 ms were applied to neonatal mouse pups. Dissected mouse eyeballs were processed as described (Chan, C. S. Y. et al. Cell type- And stage-specific expression of Otx2 is regulated by multiple transcription factors and cis-regulatory modules in the retina. Dev. 147, 1-13 (2020)). Eyeballs were fixed in 4% 702 paraformaldehyde (PFA) in 1×PBS (pH 7.4) for 2 hr at room temperature. Retinas were dissected and equilibrated at room temperature in a series of sucrose solutions (5% sucrose in 1×PBS, 5 min; 15% sucrose in 1×PBS, 15 min; 30% sucrose in 1×PBS, 1 hr; 1:1 mixed solution of OCT and 30% sucrose in PBS, 4° C., overnight), frozen and stored at −80° C. A Leica CM3050S cryostat (Leica Microsystems) was used to prepare 20 μm cryosections. Retinal cryosections were washed in 1×PBS briefly, incubated in 0.2% Triton, 1×PBS for 20 min, and blocked for 30 min in blocking solution of 0.1% Triton, 1% bovine serum albumin and 10% donkey serum (Jackson ImmunoResearch Laboratories) in 1×PBS. Slides were incubated with primary antibodies diluted in blocking solution in a humidified chamber at room temperature at 4° C. overnight. After washing in 0.1% Triton 1×PBS three times, slides were incubated with secondary antibodies and DAPI (Sigma-Aldrich; D9542) for 1-2 hr, washed three times with 0.1% Triton, 1×PBS and mounted in Fluoromount-G (Southern Biotechnology Associates). Primary antibodies for Oct4, Sox2 and Klf4 are as described in above “immunostaining” section. Additional primary antibodies used were rat anti-HA (Roche; 3F10), guinea pig anti-RBPMS (PhosphoSolutions; 1832), and rabbit anti-Pax6 (Thermo; 42-6600). Retinal slices were imaged with the LSM Confocal inverted laser scanning microscope, with Plan Apochromat objective 40×.1.4 Oil (FWD=0.13 mm) with 405, 488, 561 and 633 lasers. Quantitation was performed as described (Wang, S., Sengel, C., Emerson, M. M. & Cepko, C. L. A gene regulatory network controls the binary fate decision of rod and bipolar cells in the vertebrate retina. Dev. Cell 30, 513-527 (2014)) using Fiji software.

### Histology and Immunohistochemistry

Dissected mouse eyeballs were processed as described in Chan, C. S. Y. et al. Cell type- And stage-specific expression of Otx2 is regulated by multiple transcription factors and cis-regulatory modules in the retina, Development, 147, 1-13 (2020). Eyeballs were fixed in 4% paraformaldehyde (PFA) in 1×PBS (pH 7.4) for 2 hr at room temperature. Retinas were dissected and equilibrated at room temperature in a series of sucrose solutions (5% sucrose in 1×PBS, 5 min; 15% sucrose in 1×PBS, 15 min; 30% sucrose in 1×PBS, 1 hr; 1:1 mixed solution of OCT and 30% sucrose in PBS, 4° C., overnight), frozen and stored at −80° C. A Leica CM3050S cryostat (Leica Microsystems) was used to prepare 20 μm cryosections. Retinal cryosections were washed in 1×PBS briefly, incubated in 0.2% Triton, 1×PBS for 20 min, and blocked for 30 min in blocking solution of 0.1% Triton, 1% bovine serum albumin and 10% donkey serum (Jackson ImmunoResearch Laboratories) in 1×PBS. Slides were incubated with primary antibodies diluted in blocking solution in a humidified chamber at room temperature at 4° C. overnight. After washing in 0.1% Triton 1×PBS three times, slides were incubated with secondary antibodies and DAPI (Sigma-Aldrich; D9542) for 1-2 hr, washed three times with 0.1% Triton, 1×PBS and mounted in Fluoromount-G (Southern Biotechnology Associates). Primary antibodies for Oct4, Sox2 and Klf4 are as described in above “immunostaining” section. Additional primary antibodies used were rat anti-HA (Roche; 3F10), guinea pig anti-RBPMS (PhosphoSolutions; 1832), and rabbit anti-Pax6 (Thermo; 42-6600). Retinal slices were imaged with the LSM 710 Confocal inverted laser scanning microscope, with 20× Plan Apochromat objective (NA 0.8, wd 0.55 mm) with 405, 488, 561 and 633 lasers.

### AAV Production and Intravitreal Injection

AAV2s were produced by AAVnerGene (North Bethesda, MD) using previously described approaches (Wang, Q. et al. Mouse gamma-Synuclein Promoter-Mediated Gene Expression and Editing in Mammalian Retinal Ganglion Cells. J. Neurosci. 40, JN-RM-0102-20 (2020)). AAV titers were determined by real-time PCR. AAV-Cas12a and AAV-crYFP were mixed at a ratio of 2:1. AAV-Cas12a was diluted to 4.5×10¹² vector genome (vg)/ml and AAV-crYFP was diluted to 2.25×10¹². For intravitreal injection, mice were anesthetized by xylazine and ketamine based on their body weight (0.01 mg xylazine/g+0.08 mg ketamine/g). A pulled and polished microcapillary needle was inserted into the peripheral retina just behind the ora serrata. Approximately 2 μl of the vitreous was removed to allow injection of 2 μl AAV into the vitreous chamber to achieve 9×10⁹ vg/retina of Cas12a and 4.5×10⁹ vg/retina of crYFP. Mice were sacrificed 10 weeks after AAV injection. Transcardiac perfusion was performed as described (Wang, Q. et al. Mouse gamma-Synuclein Promoter-Mediated Gene Expression and Editing in Mammalian Retinal Ganglion Cells. J. Neurosci. 40, JN-RM-0102-20 (2020)). For retina wholemount, retinas were dissected out and washed extensively in PBS before blocking in staining buffer (10% normal goat serum and 2% Triton X-100 in PBS) for 1 h. RBPMS guinea pig antibody was made at ProSci according to publications56 and used at 1:4000, and rat HA (clone 3F10, 1:200, Roche) was diluted in the same staining buffer. Floating retinas were incubated with primary antibodies overnight at 4° C. and washed three times for 30 min each with PBS. Secondary antibodies (Cy2, Cy3, or Cy5 conjugated) were then applied (1:200; Jackson ImmunoResearch) and incubated for 1 h at room temperature. Retinas were again washed three times for 30 min each with PBS before a cover slip was attached with Fluoromount-G (SouthernBiotech). Quantitation of fluorescence of individual cells utilized a custom semi-automatic image analysis pipeline based on MATLAB (version R2019a) available at https://github.com/QilabGitHub/dCas12a-microscopy. For analysis on mouse retina wet mount, threshold-based segmentation was performed based on the fluorescent channel representing crRNA, which had highest signal-to-noise ratio and distributes evenly throughout the cytoplasm. Morphological operations were then applied to remove noise and thus yields masks for single cells. Based on the masks, mean fluorescent intensities of all corresponding channels for every cell were collected for further statistical analysis.

### Example 3: VgdCas12a Drives Superior CRISPR Activation Over Wildtype dCas12a

This Example demonstrates the superior CRISPR activation activity of VgdCas12a.

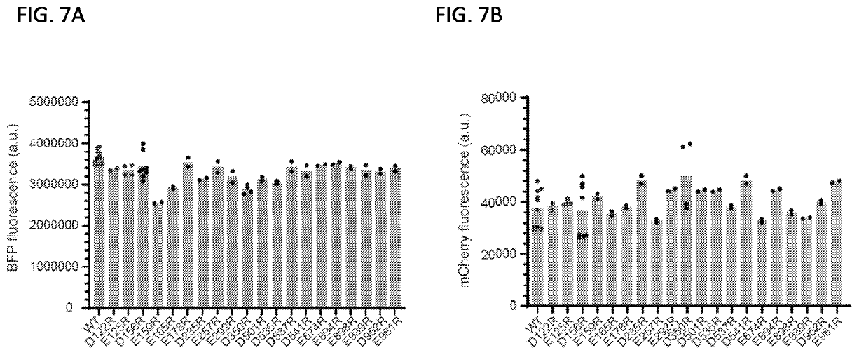

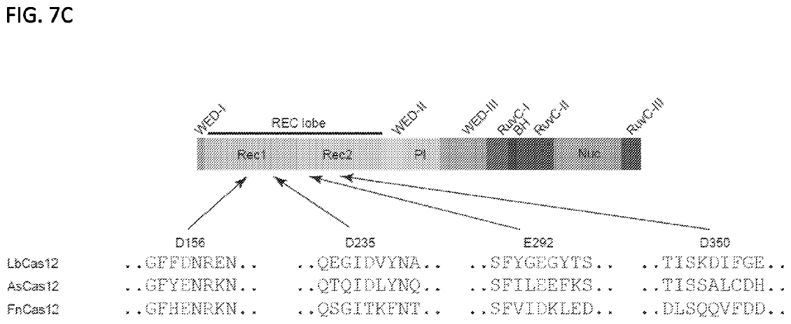

Since previous comparisons show that LbdCas12a-VPR achieves ˜5-fold higher than AsdCas12a-VPR for single-gene activation, this Example focused on LbdCas12a. A structure-guided protein engineering approach was used and focused on negatively charged (e.g., Asp or Glu) residues within LbdCas12a that reside within 10 Å of the target DNA (PDB 5XUS), and systematically mutated the negatively charged residues to positively charged arginine (FIG. 1A), with the aim of increasing affinity of the Cas protein to its target DNA. Then, these various mutants were tested in their ability to drive transcriptional activation of TRE3G-GFP in a HEK293T reporter cell line (FIG. 1B). While most mutations tested had worsened or decreased activity, a few mutants (D122R, E125R, D156R, E159R, D235R, E257R, E292R, D350R, E894R, D952R, and E981R) showed enhanced dCas12a activity (FIG. 1C and FIGS. 7A-B). Next, the effects of these mutants in a low blue fluorescent protein (BFP) bin was examined (FIG. 1D), serving as a proxy for low reactant concentrations (e.g., of crRNA and Cas12a protein), which would be particularly relevant for in vivo delivery. Notably, it was observed that several mutants exhibited even greater enhancement over WT dCas12a at lower reactant concentrations. WT dCas12a exhibited a significant decrease in activity, only enabling a ˜26-fold activation of GFP over the non-targeting control. Notably, several mutants performed substantially better than the WT protein in this condition: the single D156R mutation enabled >600-fold activation, while several others enabled 90-200-fold activation (FIG. 1D). Furthermore, the 4 best mutants (D156R, D235R, E292R, D350R) were chosen and achieved further enhancement with several permutations of combinatorial mutants (FIG. 1E).

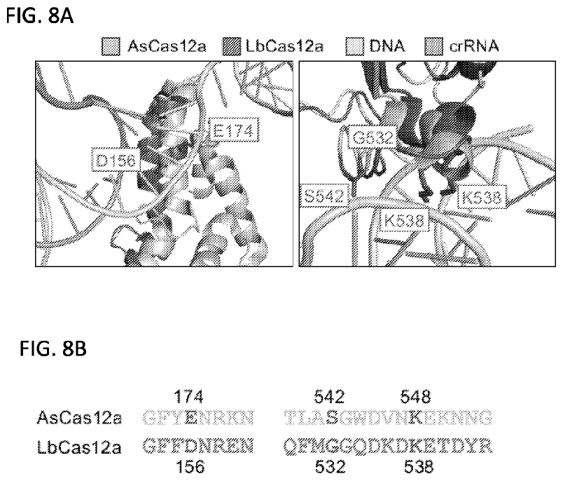

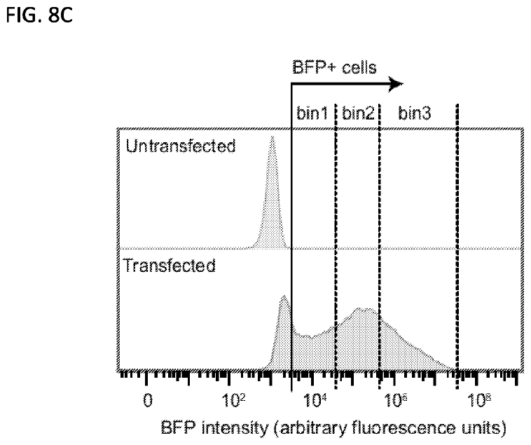

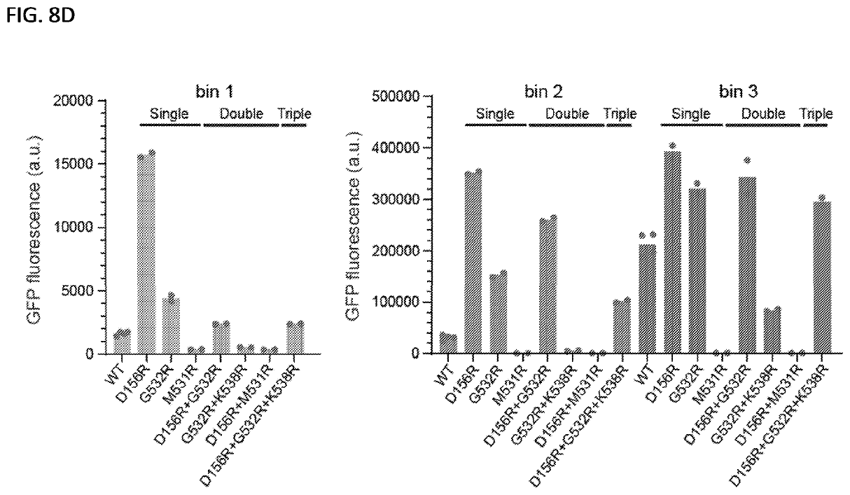

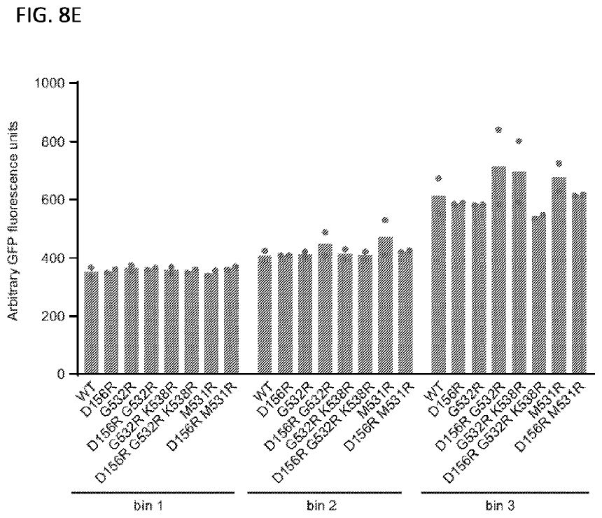

A previously reported enhanced version of Cas12a from a different species, Acidaminococcus, harbored the E174R/S542R/K548R mutations (called “enAsCas12a” and “enAsdCas12a”)). Therefore, mutations in homologous residues (D156R/S532R/K538R) in LbdCas12a were tested (FIGS. 8A-8E). Both single mutants and the triple-mutant were tested, since reports have shown utility of the single D156R mutant in plants and fungi, and its ability to enhance activity of other mutants. Interestingly, D156R combined with G532R and/or K538R did not achieve activation higher than the single D156R, in contrast to results with homologous residues in AsCas12a (FIGS. 8A-8E).

Using dCas12a for multiplex genome regulation applications would require that the protein maintains its RNAse ability to process a functional crRNA from a longer poly-crRNA transcript. To easily test this using the same GFP reporter system, we compared the performance of the dCas12a mutants to the WT protein using crRNA expressed by an RNA polymerase II promoter (CAG promoter, in this case), so that dCas12a would be required to process the crRNA before activation of the target gene. Therefore, in addition to crRNA driven by U6 promoter (FIG. 1B), the LbCas12a mutants with crRNA driven by an RNA polymerase II promoter were also tested. It is shown that the mutants described herein exhibited enhanced activation with a CAG promoter-driven crRNA (FIGS. 1F-1G). Here, GFP activation using WT dCas12a was greatly reduced using a CAG-driven crRNA compared a U6-driven crRNA (compare GFP fluorescence of WT in FIG. 1C vs. FIG. 1G), but the single and combinatorial mutants significantly enhanced the level of activation. Notably, the quadruple mutant (D156R/D235R/E292R/D350R) achieved the highest level of activation, ˜12-fold above the level achieved by the WT protein (FIG. 1G, left). We then tested the mutants in a condition with limiting crRNA quantity (a crRNA:dCas12a ratio of 0.2:1), and here, the quadruple mutant performed above all other mutants, at ˜168-fold above the level achieved by the WT protein (FIG. 1G, right). We heretofore refer to this quadruple mutant as “vgdCas12a” (very good dCas12a) for further characterization and in vivo gene targeting.

Even though the mutagenesis focused on increasing efficiency (instead of broadening targeting range as in previous studies (Kleinstiver, B. P. et al. Engineered CRISPR-Cas12a variants with increased activities and improved targeting ranges for gene, epigenetic and base editing. Nat. Biotechnol. 37, 276-282 (2019); Gao, L. et al. Engineered Cpf1 variants with altered PAM specificities. Nat. Biotechnol. 35, 789-792 (2017)), the PAM preferences of this mutant were tested specifically for gene activation. A truncated TRE3G promoter was used containing a single TetO preceded by a PAM, and it is shown that hyperdCas12a outperformed WT dCas12a for all 3 canonical PAMS (TTTA, TTTC, TTTG) as well as several of the non-canonical PAMS (TTTT, CTTA, TTCA, TTCC) (FIG. 1H). Since out of the 4 mutated residues of hyperdCas12a, only the D156R mutation is proximal to the PAM, it is logical that several of these PAMS are also accessible by the homologous E174R mutant of AsdCas12a (Kleinstiver, B. P. et al. Engineered CRISPR-Cas12a variants with increased activities and improved targeting ranges for gene, epigenetic and base editing. Nat. Biotechnol. 37, 276-282 (2019)), and that the PAM range of hyperdCas12a may be stricter than that of enAsdCas12a (Kleinstiver, B. P. et al. Engineered CRISPR-Cas12a variants with increased activities and improved targeting ranges for gene, epigenetic and base editing. Nat. Biotechnol. 37, 276-282 (2019)).

### Example 4: VgdCas12a Outperforms WT dCas12a for Gene Editing, CRISPR Repression, and Base Editing

This Example demonstrates that the vgdCas12a is useful for additional Cas12a-based applications, including CRISPR repression and base editing. Additionally, this Examples shows that the four activity-enhancing mutations, when introduced into the nuclease-active form of Cas12a, enhanced gene editing.

First, the four activity-enhancing mutations were introduced into the nuclease-active form of Cas12a, and it was shown that the vgCas12a (very good Cas12a) enabled more effective GFP knockout in SV40-GFP reporter cells (FIGS. 2A-2B).

Furthermore, vgdCas12a can be modularly coupled to different effectors and exhibit enhanced regulatory effects. For example, when coupled to a transcriptional repressor, the mutant fusion protein showed 2 to 3-fold improvement compared to the wildtype fusion protein (FIGS. 2C-2D).

VgdCas12a, when coupled to the A-to-G base editor ABE8, substantially improved base editing in a reporter system where A-to-G editing of an internal stop codon results in a functional GFP protein (FIG. 2E-G), and also improved base editing of an endogenous gene target (FIG. 2H). Additionally, it was shown in a “dual reporter” system that translation of a full-length GFP protein requires simultaneous targeting by two crRNAs (FIG. 2I-J), indicating the high specificity of base editing by ABE8.

To test gene editing in vivo, hyperCas12a was packaged in an adenovirus-associated virus 141 (AAV) serotype 2 with a retinal ganglion cell-specific promoter further miniaturized from a previous study (Wang, Q. et al. Mouse gamma-Synuclein Promoter-Mediated Gene Expression and Editing in Mammalian Retinal Ganglion Cells. J. Neurosci. 40, JN-RM-0102-20 (2020)) (265 bp), a truncated WPRE (245 bp) (Levy, J. M. et al. Cytosine and adenine base editing of the brain, liver, retina, heart and skeletal muscle of mice via adeno-associated viruses. Nat. Biomed. Eng. 4, 97-110 (2020)), and a small synthetic poly-A tail (49 bp) (FIG. 2K). In transgenic mice expressing Thy1-YFP (Feng, G. et al. Imaging Neuronal Subsets in Transgenic Mice Expressing Multiple Spectral Variants of GFP. Neuron 28, 41-51 (2000)), AAV-hyperCas12a was co-delivered by intravitreal injection along with AAV-crRNA (YFP) in one eye, and its wildtype counterpart in the contralateral eye as a side-by-side control (FIG. 2L). For all mice tested, hyperCas12a showed improved YFP knockout compared to WT Cas12a (FIGS. 2M-20). Despite using minimal versions of all regulatory elements, the AAV containing hyperdCas12a (4743 bp) nonetheless teetered on the AAV packaging limit (˜4.7 bp); by being 234 bp larger, enAsdCas12a exceeded this limit (FIG. 2K). This highlights the utility of hyperCas12a for enhanced AAV-based in vivo gene-editing.

### Example 5: CRISPR Activation by vgdCas12a is Highly Specific

This Example evaluates the specificity of CRISPR activation by vgdCas12a on a genome-wide scale, and demonstrates that CRISPR activation by vgdCas12a described herein is highly specific.

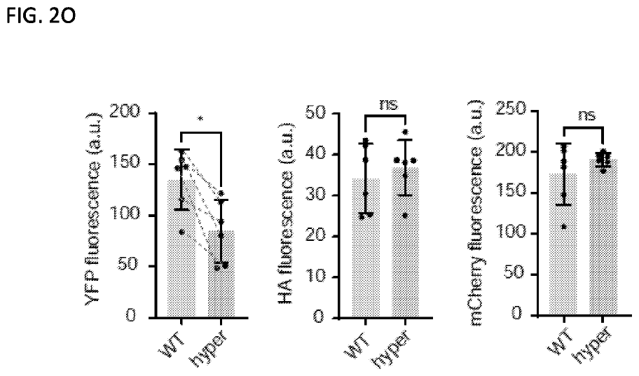

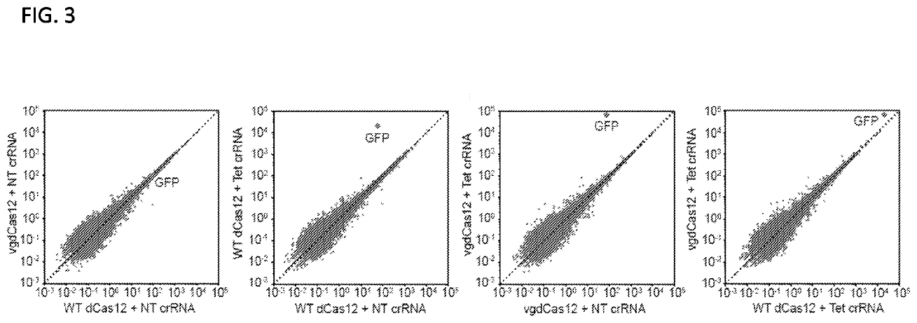

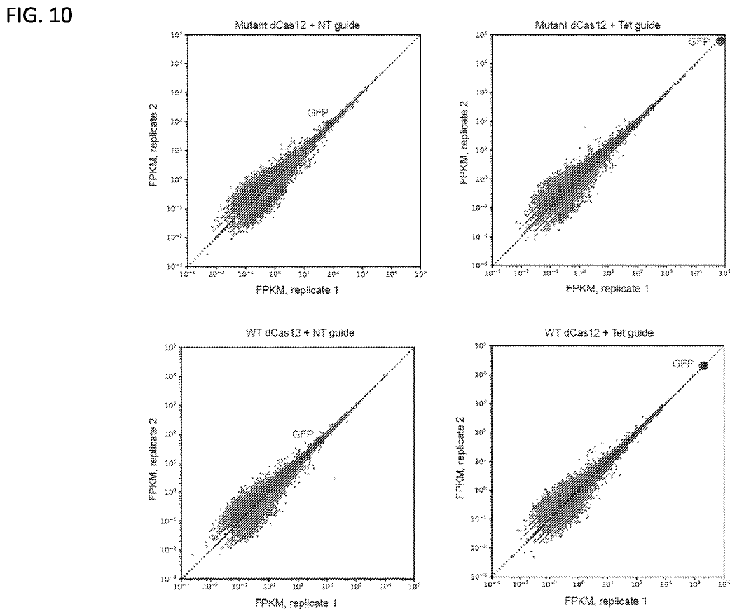

To evaluate the specificity of CRISPR activation by vgdCas12a on a genome-wide scale, we carried out whole-transcriptome RNA-seq of HEK293T cells with the TRE3G-GFP reporter (FIG. 1B) transfected with either WT dCas12a or vgdCas12a combined with the TRE3G-targeting crRNA (FIG. 3). We also included a non-targeting crRNA as negative control for each case. Two biological replicates were analyzed separately and showed similar results (FIG. 10). As expected, with the targeting crRNA, the GFP transcript exhibited an increase in abundance, consistent with flow cytometry data showing stronger transcriptional activation by vgdCas12a compared to the WT dCas12a in FIG. 1C (FIG. 3). Comparing the targeting vs. non-targeting crRNAs, both WT dCas12a and vgdCas12a showed similar specificity, and no genes were observed with significantly altered expression (FIG. 3). These plots together demonstrate that vgdCas12a exhibits comparable specificity as WT dCas12a.

### Example 6: VgdCas12a Effectively Activates Endogenous Genes

This Example shows that the VgdCas12a described herein effectively activates endogenous genes and exhibits synergistic endogenous gene activation.

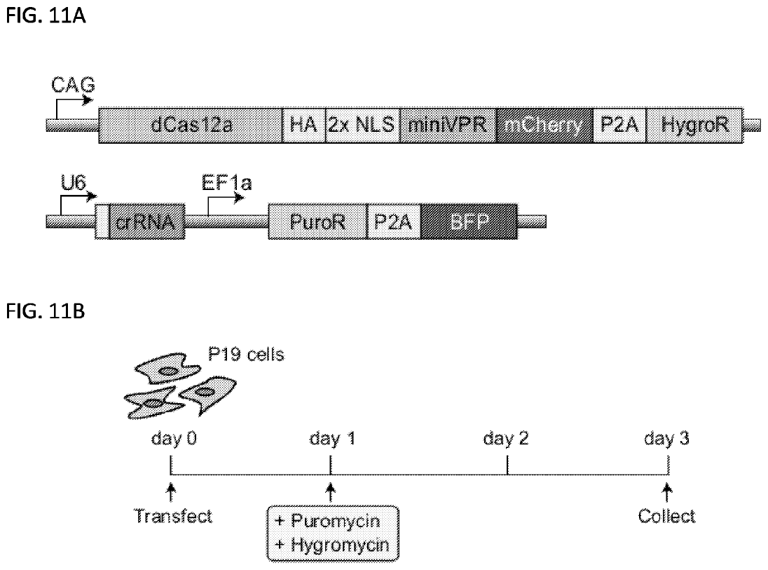

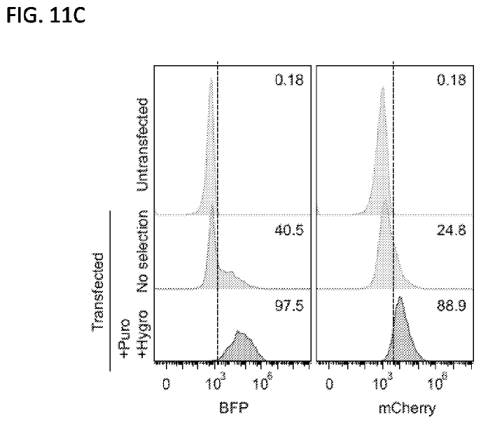

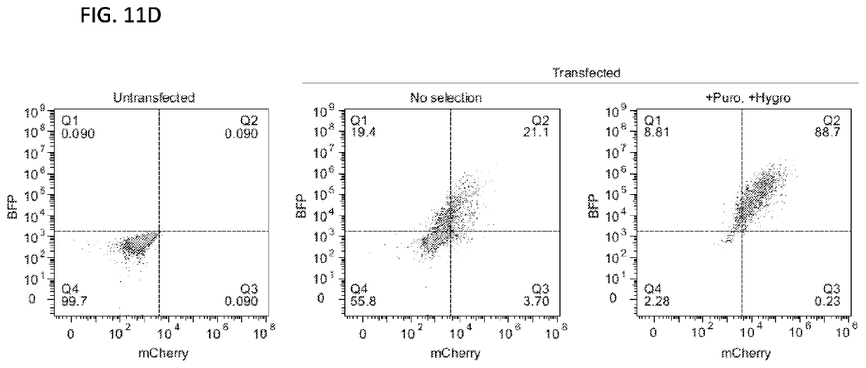

Next, the testing moved beyond the GFP reporter cell line to endogenous genes activating. Mouse P19 cells were used, in which ˜21% transfection efficiency of the two plasmids was achieved (FIG. 11A-D). Nonetheless, since the ˜21% transfection efficiency is still too low for interpretation of bulk measurements, a dual-selection approach was used. In brief, the cells were treated at 24 hr after transfection with both puromycin and hygromycin for 48 hours (FIG. 4A), which resulted in ˜89% double-positive cells (FIG. 11A-D). This dual-selection approach allowed facile comparisons between different crRNAs as well as different dCas12a mutants, compared to alternative strategies of packing different lentivirus or making numerous stable cell lines.

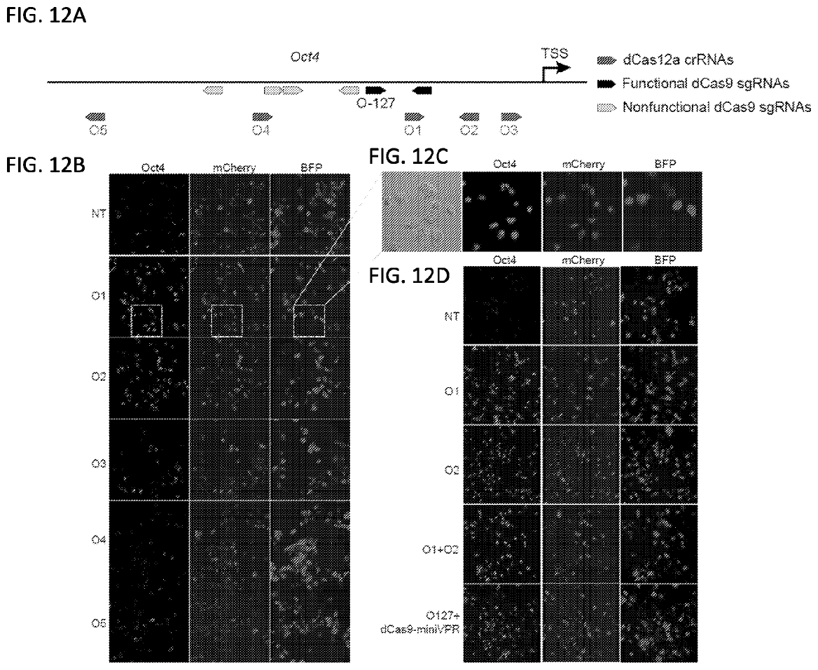

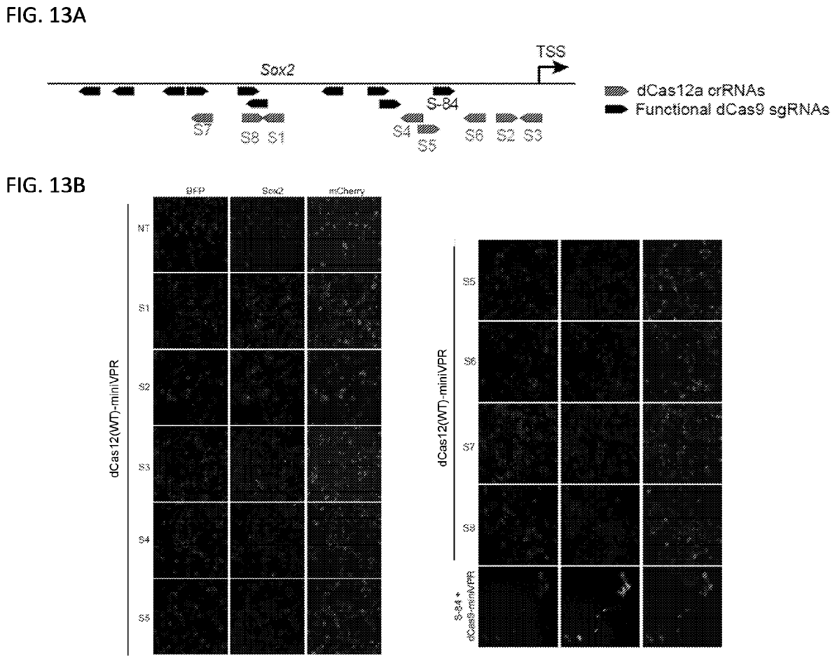

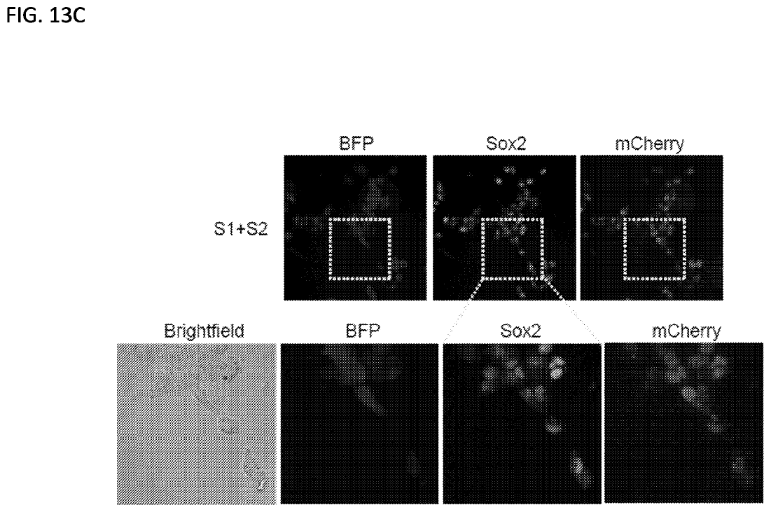

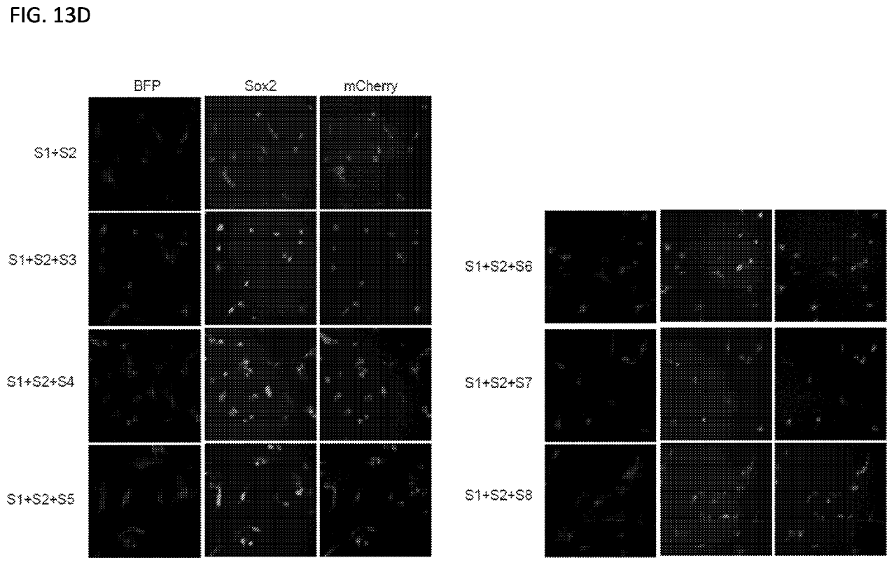

CrRNAs targeting promoters of the transcription factors Oct4, Sox2, and Klf4 were tested, given their known synergistic regenerative role in multiple contexts. Cas12a crRNAs targeting the promoter of each gene were designed (FIG. 12-14, Table 2), encompassing regions previously targeted by dCas9-SunTag-VP64 in mouse embryonic stem cells. Immunostaining was used to visualize target protein expression in cells, and to identify several crRNAs that effectively enabled transcriptional activation of Oct4 (FIG. 12), Sox2 (FIG. 13), and Klf4 (FIG. 14). Furthermore, for Sox2 and Klf4, synergistic activation was achieved by using paired crRNAs (even though target sequences for Klf4 crRNAs were >500 nt apart), and further synergy in Sox2 activation was achieved by using a “triplet” of three separate Sox2 crRNAs (FIG. 13-14). Using a subset of the validated crRNAs, the level of endogenous gene activation was compared between WT dCas12a vs. vgdCas12a. All crRNAs tested, including paired and triplet crRNAs, exhibited enhanced activation using vgdCas12a compared to WT dCas12a (FIG. 4B-D).

TABLE 2
crRNA sequences

| Target | Name | Sequence (SEQ ID NO) |
|---|---|---|
| Tet | CrTet | CTCCCTATCAGTGATAGAGAACG |
| | | (SEQ ID NO: 9) |
| LacZ | CrLacZ | CGAATACGCCCACGCGATGGGT |
| | | (SEQ ID NO: 10) |
| Sox2 | S1 | AGCAACAGGTCACGGCGCACG |
| promoter | | (SEQ ID NO: 11) |
| | S2 | TTCCCTGACAGCCCCCATCACAT |
| | | (SEQ ID NO: 12) |
| | S3 | AACAAGTTAATAGACAACCATCC |
| | | (SEQ ID NO: 13) |
| | S4 | CATGAAAGGGGGCGGGGCCT |
| | | (SEQ ID NO: 14) |
| | S5 | ATGCAAAACCCTCTGGCGAG |
| | | (SEQ ID NO: 15) |
| | S6 | CGGCGGCCAATCAGCGAGCG |
| | | (SEQ ID NO: 16) |
| | S7 | CCCCATGCTACGGAATATTGGCT |
| | | (SEQ ID NO: 17) |
| | S8 | CCCACTTCCTTCGAACAGGCGTG |
| | | (SEQ ID NO: 18) |
| Oct4 | O1 | ACCTCTCCCTCCCCAATCCCACC |
| promoter | | (SEQ ID NO: 19) |
| | O2 | CACCAGGCCCCCGGCTCGGGGTG |
| | | (SEQ ID NO: 20) |
| | O3 | AGCCCATGTCCAAGGCCAGGACA |
| | | (SEQ ID NO: 21) |
| | O4 | TGGTGCGATGGGGCATCCGAGCA |
| | | (SEQ ID NO: 22) |
| | O5 | TCCCACCCCCACAGCTCTGCTCC |
| | | (SEQ ID NO: 23) |
| Klf4 | K1 | ATAGCAGGCGCGGAACCCCTTCT |
| promoter | | (SEQ ID NO: 24) |
| | K2 | GCTACCATGGCAACGCGCAGTGG |
| | | (SEQ ID NO: 25) |
| | K3 | GAGCCCAGGGAACCGACCGTGGC |
| | | (SEQ ID NO: 26) |
| | K4 | TTCTCCCCGCCTTCCCGCAGCCC |
| | | (SEQ ID NO: 27) |

### Example 7: VgdCas12a Drives Enhanced Multiplex Activation of Endogenous Targets

This Example demonstrates the enhanced multiplex activation of endogenous genes driven by the vgdCas12a described herein.

Cas12a possesses both DNAse and RNAse activities and controls the processing and maturation of its own crRNA in addition to editing its target genes. Engineered Cas12a systems are transcribed as a long RNA transcript (called pre-crRNA) consisting of direct repeats (DRs). Since Oct4, Sox2, and Klf4 are known to work synergistically, there is strong rationale for their multiplex activation. With best crRNAs identified to the three target genes, a single crRNA array driven by the U6 promoter encoding 6 crRNAs was co-expressed to activate the three endogenous genes (FIG. 4E). DCas12a(D156R) and a double mutant (D156R+E292R) achieved significantly enhanced activation over WT dCas12a, and further enhancement was achieved by vgdCas12a which reached ˜5-fold activation of Oct4, ˜8-fold activation of Sox2, and ˜70-fold activation of Klf4 (FIG. 4F). Of note, hyperdCas12a also outperformed enAsdCas12a (FIG. 4I). Interestingly, vgdCas12a achieved this compelling Oct4 activation in P19 cells despite its location as the 6^{th} crRNA, despite prior studies with WT dCas12a showing decreased expression of crRNAs at and beyond the 4^{th} position. The activation of each target gene is decreased compared to the level achieved by single crRNAs (compare FIG. 4F to FIGS. 4B-4D), likely due to decreased copies of the longer pre-crRNA array expressed by the U6 promoter compared to shorter individual crRNAs. Nevertheless, vgdCas12a performed robustly in using a single CRISPR array to activate multiple endogenous targets. Additionally, the enhanced performance of vgdCas12a over the single D156R mutant and the double D156R/E292R mutant in this assay highlights the synergistic power of these combinatorial mutations, and points to vgdCas12a as a logical protein of choice for multiplex genome engineering in mammalian cells.

### Example 8: In Vivo Multiplex Activation by vgdCas12a in Mouse Retina Directs Progenitor Cell Differentiation

This Example demonstrates the in vivo multiplex activation by vgdCas12a described herein in mouse retina directs retinal progenitor cell differentiation.

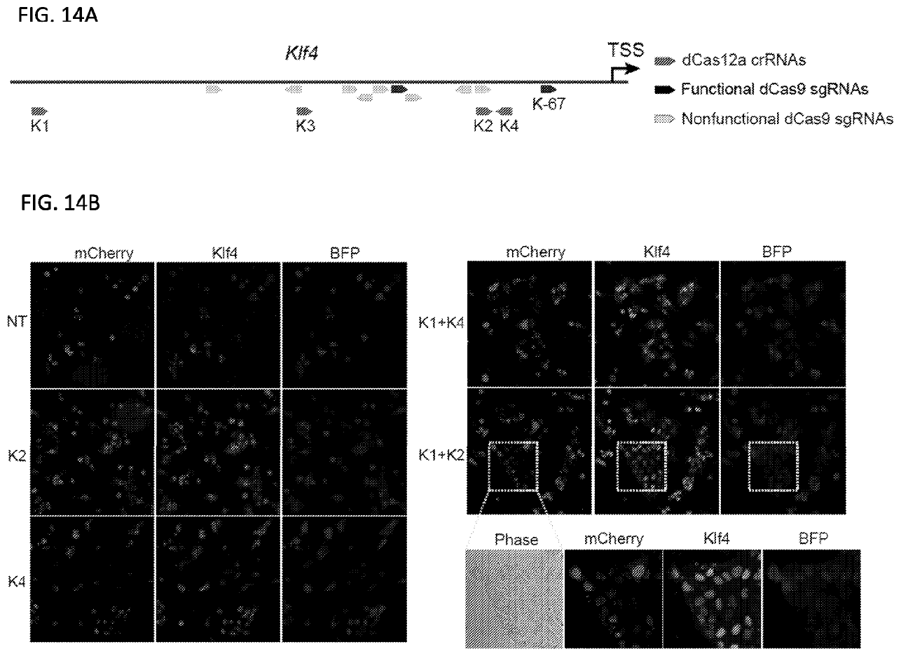

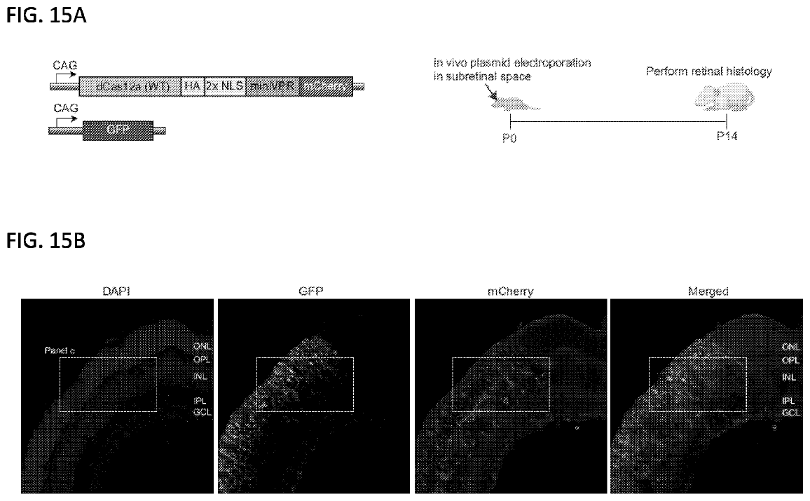

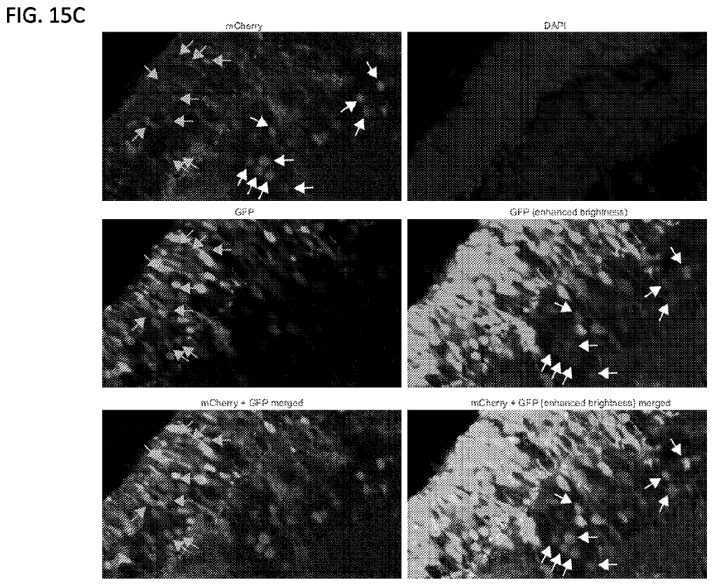

The retina was targeted for in vivo applications given the high interest in using genome engineering for eye disease, its relative immune privilege and accessibility, and the global burden of degenerative retinal diseases. The well-validated in vivo electroporation technique was used, which has several advantages over other methods of gene transfer, such as more lenient size limitation of the transgene. Transgenes persist up to a few months in retina cells in vivo. In vivo electroporation allowed expression of the full-length WT dCas12a at 14 days after delivery, which exhibited high expression both in the outer nuclear layer (ONL, consisting of rod and cone photoreceptors) and in the inner nuclear layer (INL, consisting of amacrine, bipolar, horizontal neurons, as well as Müller glia) (FIG. 15).

The effect of multiplex CRISPR activation in the retina was tested as proof of principle of the vgdCas12a system. Overexpression of Sox2, Oct4 and Klf4 individually have been shown to redirect the differentiation of retinal progenitor cells (RPCs) towards specific fates, but their potential for retinal reprogramming and rejuvenation has not been fully elucidated. Since synergistic co-activation of these three transcription factors can induce the formation of iPSCs in vitro and rejuvenate mature retinal ganglion cells for regeneration in vivo, it was tested whether the vgdCas12a system can synergistically activate Sox2, Klf4 and Oct4 in postnatal RPCs in vivo, and whether this manipulation affects the differentiation capacity of RPCs.

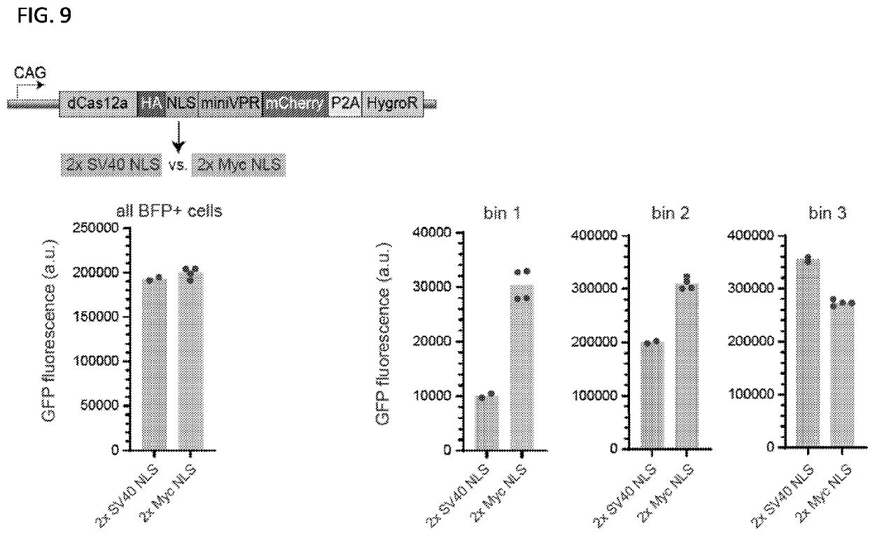

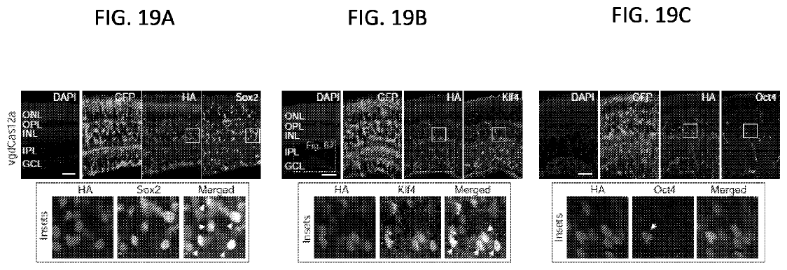

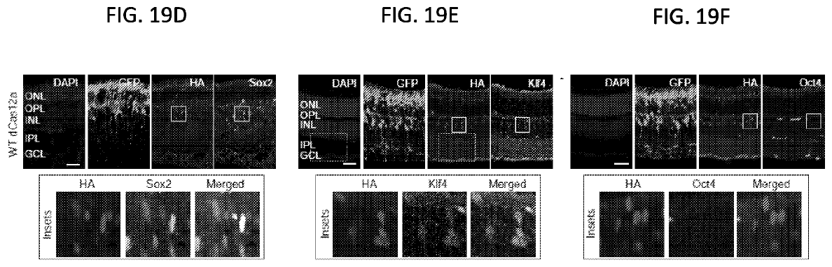

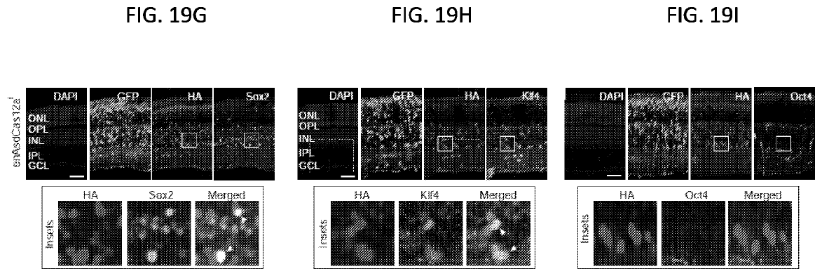

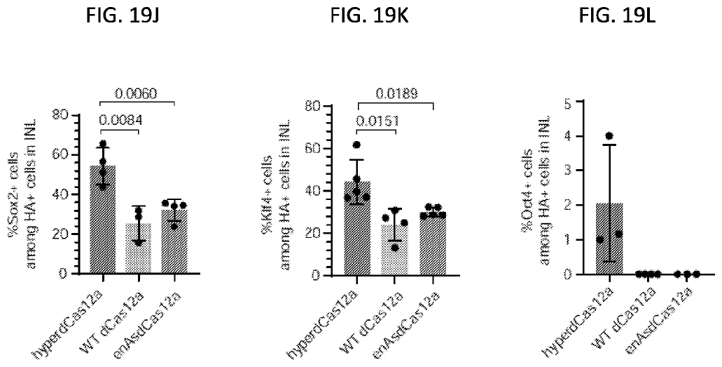

A single plasmid consisting of HA-tagged vgdCas12a was constructed with an optimized nuclear-targeting sequence (NLS) structure (FIG. 9) and a poly-crRNA targeting Sox2, Klf4, and Oct4, and was delivered this into the mouse retina in vivo via electroporation at postnatal day 0 (P0). The CAG-GFP plasmid was co-electroporated to serve as electroporation efficiency control. Within the electroporated GFP+ patches in the retina, numerous HA+ cells were observed, indicating successful delivery and expression of vgdCas12a (FIGS. 5-6, 16). While Sox2, Klf4 and Oct 4 were not activated by nontargeting control crRNA, strong expression of Klf4 (FIG. 5B-C) and Sox2 (FIG. 5D-E) were observed, as well as weak activation of Oct4 in HA+ cells (FIG. 17), indicating successful CRISPR activation of these targets. Further, the level of in vivo activation of all three gene targets was stronger with hyperdCas12a (FIGS. 19A-19C) than with WT dCas12a (FIGS. 19D-19F, 19J-19L), enAsdCas12a (FIGS. 19G-19I, 19J-19L), which is consistent with the in vitro results (FIG. 4I).

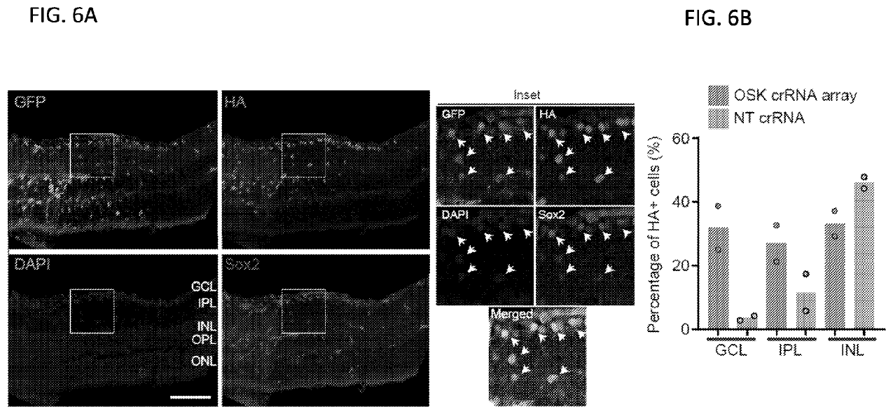

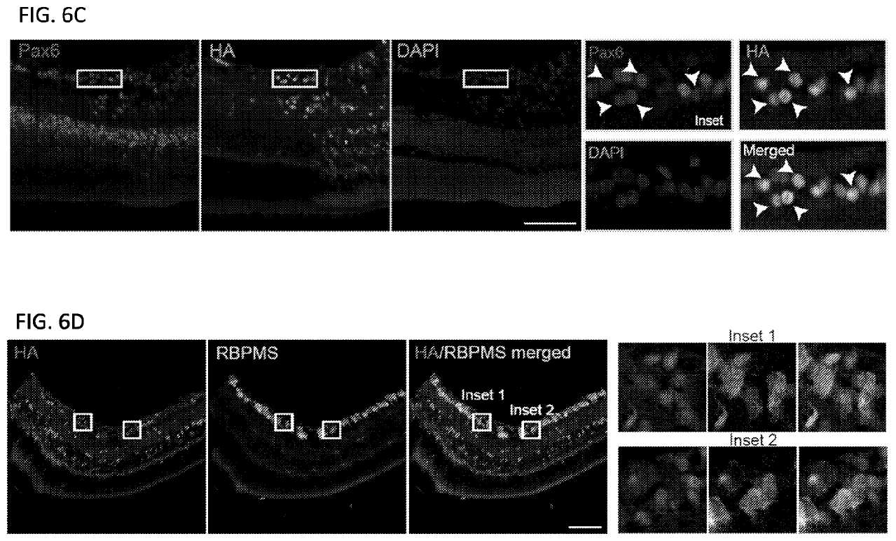

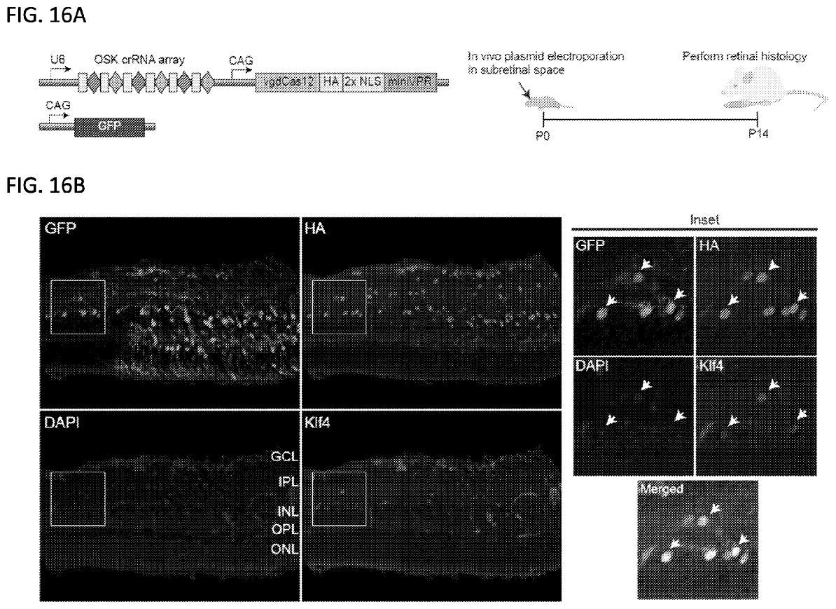

The fates of HA+ cells that have received the vgdCas12a and poly-crRNA array plasmid were examined. The in vivo electroporation technique delivers DNA mainly to mitotic cells, and at postnatal day 0, mitotic RPCs give rise to rod photoreceptors, Müller glia, and bipolar and amacrine neurons, which migrate to and reside in the ONL (outer nuclear layer) or INL (inner nuclear layer), but not in GCL (ganglion cell layer). It was noted that activation by vgdCas12a-miniVPR with the crRNA array resulted in a strong population of HA+/Sox2+/Klf4+ cells in GCL and inner plexiform layer (IPL), which were not seen in non-targeting controls (FIGS. 6A-6B, and 16). It is likely that CRISPRa of Sox2/Klf4 in P0 RPCs induced migration of cells into the GCL. In most of the HA+ cells that migrated into GCL, we observed expression of Pax6 (marker for retinal displaced amacrine and ganglion cells in GCL) but not RBPMS (marker for retinal ganglion cells) (FIG. 6C). However, a minority of GCL HA+ cells expressed RBPMS (FIG. 6D). These data suggest that transcriptional activation of Sox2 and Klf4 (and weakly, Oct4) can reprogram postnatal RPCs to differentiate into displaced amacrine-like and ganglion-like cells, and support the conclusion that the engineered vgdCas12a variant can activate multiple endogenous genes in vivo to induce significant organismal phenotypes for in vivo research.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

All publications, published patent documents, and patent applications cited herein are hereby incorporated by reference to the same extent as though each individual publication, published patent document, or patent application was specifically and individually indicated as being incorporated by reference.

What is claimed is:

1. An engineered Cluster Regularly Interspaced Short Palindromic Repeat (CRISPR)-associated (Cas) 12a protein, comprising a sequence that is at least 80% identical to the amino acid sequence of SEQ ID NO: 1 or 2, wherein the engineered Cas12a protein comprises one or more mutations selected from the list consisting of D122R, E125R, D156R, E159R, D235R, E257R, E292R, D350R, E894R, D952R, and E981R.

2. The engineered Cas12a protein of claim 1, wherein the engineered Cas12a protein comprises one or more mutations selected from the list consisting of D156R, D235R, E292R, and D350R.

3. The engineered Cas12a protein of claim 1 or 2, wherein the engineered Cas12a protein comprises at least two, three, or four mutations.

4. The engineered Cas12a protein of any one of the preceding claims, wherein the engineered Cas12a protein comprises the mutations of D156R and E292R.

5. The engineered Cas12a protein of any one of the preceding claims, wherein the engineered Cas12a protein comprises the mutations of D156R and D350R.

6. The engineered Cas12a protein of any one of the preceding claims, wherein the engineered Cas12a protein comprises the mutations of D156R, E292R, and D122R.

7. The engineered Cas12a protein of any one of the preceding claims, wherein the engineered Cas12a protein comprises the mutations of D156R, E292R, and D235R.

8. The engineered Cas12a protein of any one of the preceding claims, wherein the engineered Cas12a protein comprises the mutations of D156R, E292R, and D350R.

9. The engineered Cas12a protein of any one of the preceding claims, wherein the engineered Cas12a protein comprises the mutations of D156R, D235R, E292R, and D350R.

10. The engineered Cas12a protein of any one of the preceding claims, wherein the engineered Cas12a protein exhibits improved activation compared to the wild type (WT) Cas12a protein.

11. The engineered Cas12a protein of any one of the preceding claims, wherein the engineered Cas12a protein exhibits improved repression compared to the wild type (WT) Cas12a protein.

12. The engineered Cas12a protein of any one of the preceding claims, wherein the engineered Cas12a protein exhibits enhanced regulatory effect compared to the WT Cas12a protein.

13. The engineered Cas12a protein of any one of the preceding claims, wherein the engineered Cas12a protein exhibits improved epigenetic modifications compared to the wild type (WT) Cas12a protein.

14. The engineered Cas12a protein of any one of the preceding claims, wherein the engineered Cas12a protein exhibits improved gene knockout, knockin, and mutagenesis compared to the wild type (WT) Cas12a protein.

15. The engineered Cas12a protein of any one of the preceding claims, wherein the engineered Cas12a protein exhibits improved gene editing of single or multiple bases compared to the wild type (WT) Cas12a protein.

16. The engineered Cas12a protein of any one of the preceding claims, wherein the engineered Cas12a protein exhibits improved gene prime editing compared to the wild type (WT) Cas12a protein.

17. The engineered Cas12a protein of any one of the preceding claims, wherein the engineered Cas12a protein is less susceptibility to variations in crRNA concentration compared to the WT Cas12a protein.

18. The engineered Cas12a protein of any one of the preceding claims, wherein the engineered Cas12a protein exhibits increased level of activation under crRNA:Cas12a ratio of or lower compared to the WT Cas12a protein.

19. A nucleic acid encoding the engineered Cas12a protein of any one of the preceding claims.

20. A vector comprising the nucleic acid of claim 19.

21. The vector of claim 20, further comprising a promoter.

22. An engineered Cluster Regularly Interspaced Short Palindromic Repeat (CRISPR)-associated (Cas) 12a system comprising: (a) one or more CRISPR RNAs (crRNAs) or a nucleic acid encoding each of the one or more crRNAs; and (b) the engineered Cas12a protein of any one of the preceding claims or a nucleic acid encoding the engineered Cas12a protein thereof.

23. The engineered Cas12a system of any one of the preceding claims, wherein each of the one or more crRNAs comprises a repeat sequence and a spacer.

24. The engineered Cas12a system of any one of the preceding claims, wherein each spacer is configured to hybridize to a target nucleic acid.

25. The engineered Cas12a system of any one of the preceding claims, wherein each spacer in at least a portion of the one or more crRNAs is configured to hybridize to the same target nucleic acid.

26. The engineered Cas12a system of any one of the preceding claims, wherein each spacer in at least a portion of the one or more crRNAs is configured to hybridize to a different target nucleic acid.

27. The engineered Cas12a system of any one of the preceding claims, wherein each spacer in all of the one or more crRNAs is configured to hybridize to a different target nucleic acid.

28. The engineered Cas12a system of any one of the preceding claims, wherein the target nucleic acid is a DNA.

29. The engineered Cas12a system of any one of the preceding claims, wherein the system comprises one or more expression vectors.

30. The engineered Cas12a system of any one of the preceding claims, wherein the one or more crRNAs and the engineered Cas12a protein are located in separate vectors.

31. The engineered Cas12a system of any one of the preceding claims, wherein the one or more crRNAs and the engineered Cas12a protein are located in the same vector.

32. The engineered Cas12a system of any one of the preceding claims, wherein the expression of the one or more crRNAs or the engineered Cas12a protein is driven by an RNA polymerase III promoter or an RNA polymerase II promoter.

33. The engineered Cas12a system of any one of the preceding claims, wherein the RNA polymerase III promoter comprises the mouse U6 promoter, the human U6 promoter, the H1 promoter, and the 7SK promoter.

34. The engineered Cas12a system of any one of the preceding claims, wherein the RNA polymerase II promoter comprises a CAG promoter, PGK promoter, CMV promoter, EF1α promoter, SV40 promoter, and Ubc promoter.

35. The engineered Cas12a system of any one of the preceding claims, wherein the CAG promoter is synthetic.

36. The engineered Cas12a system of any one of the preceding claims, wherein the expression of the one or more crRNAs or the engineered Cas12a protein is driven by an inducible promoter.

37. The engineered Cas12a system of claim 36, wherein the inducible promoter comprises a TRE promoter.

38. The engineered Cas12a system of any one of the preceding claims, wherein the one or more crRNAs and the engineered Cas12a protein are located in the same vector, and wherein the expression of the one or more crRNAs or the engineered Cas12a protein is driven by the same promoter.

39. The engineered Cas12a system of any one of the preceding claims, wherein the one or more crRNAs and the engineered Cas12a protein are located in the same vector, and wherein the expression of the one or more crRNAs or the engineered Cas12a protein is driven by different promoters.

40. A method of modulating one or more target nucleic acids in a sample, comprising contacting the sample with a plurality of the engineered Cas12a protein, or a plurality of the engineered Cas12a system, of any one of the preceding claims.

41. The method of claim 40, comprising modulating the more than one target nucleic acids simultaneously.

42. The method of any one of the preceding claims, wherein the modulating results in transcriptional activation of the one or more target nucleic acids.

43. The method of any one of the preceding claims, wherein the modulating results in transcriptional repression of the one or more target nucleic acids.

44. The method of any one of the preceding claims, wherein the modulating results in epigenetic modifications including targeted CpG methylation, histone H2, H3 or H4 methylation or acetylation of the one or more target nucleic acids.

45. The method of any one of the preceding claims, wherein the modulating results in editing single or multiple bases of the one or more target nucleic acids.

46. The method of any one of the preceding claims, wherein the modulating results in altered expression of the one or more target nucleic acids.

47. The method of any one of the preceding claims, wherein the modulating results in reprograming the lineage of the sample.

48. The method of any one of the preceding claims, wherein the modulating the target nucleic acid in the sample results in depletion of the one or more target nucleic acids.

49. The method of any one of the preceding claims, wherein the one or more target nucleic acids comprise one or more nucleic acids encoding functional proteins.

50. The method of any one of the preceding claims, wherein the one or more target nucleic acids comprise one or more nucleic acids encoding transcriptional factors and/or metabolic enzymes.

51. The method of any one of the preceding claims, wherein the one or more target nucleic acids is derived from the genomic DNA, mitochondria DNA, chloroplast DNA, or viral DNA in host cells.

52. The method of any one of the preceding claims, wherein the sample comprises one or more cells.

53. The method of any one of the preceding claims, wherein the contacting takes place in vitro or in vivo.

54. A pharmaceutical composition comprising the engineered Cas12a protein, the nucleic acid, or the vector of any one of the preceding claims.

55. A pharmaceutical composition comprising the engineered Cas12a system of any one of the preceding claims.

56. The pharmaceutical composition of any one of the preceding claims, further comprising one or more pharmaceutically acceptable excipient.

57. A method for treating a disorder in an individual in need thereof, comprising administering a therapeutically effective dose of the pharmaceutical composition of any one of the preceding claims.

58. The method of claim 57, wherein the disorder is monogenic or polygenic.

59. The method of claim 57 or 58, wherein the disorder comprises an inherited retinal degenerative disorder, an inherited optic nerve disorder, and a polygenic degenerative disease of the eye.

60. The method of claim 59, wherein the inherited retinal degenerative disorder comprises Leber's congenital amaurosis and retinitis pigmentosa.

61. The method of claim 59, wherein the inherited optic nerve disorder comprises Leber's hereditary optic neuropathy and autosomal dominant optic neuropathy.

62. The method of claim 59, wherein the polygenic degenerative disease of the eye comprises glaucoma and macular degeneration.


SEQUENCE LISTING (27 sequences)

SEQ ID NO: 1 - protein, 1228 aa, Artificial Sequence
  Synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1228 aa, Artificial Sequence
  Synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 3 - DNA, 3684 bp, Artificial Sequence
  Synthetic construct
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgccaggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 4 - DNA, 3684 bp, Artificial Sequence
  Synthetic construct
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 5 - protein, 1228 aa, Artificial Sequence
  Synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIRVYNAIIGGFVTESGEKIKGLNEYINLYNQKTKQKLPKFKPLYKQVLSDRESLSFYGRGYTSDEEVLEVFRNTLNKNSEIFSSIKKLEKLFKNFDEYSSAGIFVKNGPAISTISKRIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 6 - protein, 1228 aa, Artificial Sequence
  Synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIRVYNAIIGGFVTESGEKIKGLNEYINLYNQKTKQKLPKFKPLYKQVLSDRESLSFYGRGYTSDEEVLEVFRNTLNKNSEIFSSIKKLEKLFKNFDEYSSAGIFVKNGPAISTISKRIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 7 - DNA, 3684 bp, Artificial Sequence
  Synthetic construct
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttcgtaacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatccgcgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcaggggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaagcgtatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgccaggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 8 - DNA, 3684 bp, Artificial Sequence
  Synthetic construct
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttcgtaacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatccgcgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcaggggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaagcgtatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 9 - DNA, 23 bp, Artificial Sequence
  Synthetic construct
ctccctatcagtgatagagaacg

SEQ ID NO: 10 - DNA, 22 bp, Artificial Sequence
  Synthetic construct
cgaatacgcccacgcgatgggt

SEQ ID NO: 11 - DNA, 21 bp, Artificial Sequence
  Synthetic construct
agcaacaggtcacggcgcacg

SEQ ID NO: 12 - DNA, 23 bp, Artificial Sequence
  Synthetic construct
ttccctgacagcccccatcacat

SEQ ID NO: 13 - DNA, 23 bp, Artificial Sequence
  Synthetic construct
aacaagttaatagacaaccatcc

SEQ ID NO: 14 - DNA, 20 bp, Artificial Sequence
  Synthetic construct
catgaaagggggcggggcct

SEQ ID NO: 15 - DNA, 20 bp, Artificial Sequence
  Synthetic construct
atgcaaaaccctctggcgag

SEQ ID NO: 16 - DNA, 20 bp, Artificial Sequence
  Synthetic construct
cggcggccaatcagcgagcg

SEQ ID NO: 17 - DNA, 23 bp, Artificial Sequence
  Synthetic construct
ccccatgctacggaatattggct

SEQ ID NO: 18 - DNA, 23 bp, Artificial Sequence
  Synthetic construct
cccacttccttcgaacaggcgtg

SEQ ID NO: 19 - DNA, 23 bp, Artificial Sequence
  Synthetic construct
acctctccctccccaatcccacc

SEQ ID NO: 20 - DNA, 23 bp, Artificial Sequence
  Synthetic construct
caccaggcccccggctcggggtg

SEQ ID NO: 21 - DNA, 23 bp, Artificial Sequence
  Synthetic construct
agcccatgtccaaggccaggaca

SEQ ID NO: 22 - DNA, 23 bp, Artificial Sequence
  Synthetic construct
tggtgcgatggggcatccgagca

SEQ ID NO: 23 - DNA, 23 bp, Artificial Sequence
  Synthetic construct
tcccacccccacagctctgctcc

SEQ ID NO: 24 - DNA, 23 bp, Artificial Sequence
  Synthetic construct
atagcaggcgcggaaccccttct

SEQ ID NO: 25 - DNA, 23 bp, Artificial Sequence
  Synthetic construct
gctaccatggcaacgcgcagtgg

SEQ ID NO: 26 - DNA, 23 bp, Artificial Sequence
  Synthetic construct
gagcccagggaaccgaccgtggc

SEQ ID NO: 27 - DNA, 23 bp, Artificial Sequence
  Synthetic construct
ttctccccgccttcccgcagccc